(19) United States
(12) Patent Application Publication
(10) Pub. No.: US 2016/0311908 A1
(43) Pub. Date: Oct. 27, 2016

(54) METHODS OF TREATING PATIENTS HAVING MUTATIONS IN THE EXTRACELLULAR DOMAIN OF EPIDERMAL GROWTH FACTOR RECEPTOR (EGFR) WITH A COMBINATION OF THREE FULLY HUMAN MONOCLONAL ANTI-EGFR ANTIBODIES

(71) Applicant: Merrimack Pharmaceuticals, Inc., Cambridge, MA (US)

(72) Inventors: Sabrina ARENA, Torino (IT); Alberto BARDELLI, Torino (IT); Jeffrey David KEARNS, Arlington, MA (US); Beni B. WOLF, Lexington, MA (US); Rachel C. NERING, Stoneham, MA (US); Hongfang WANG, Malden, MA (US)

(21) Appl. No.: 15/137,081

(22) Filed: Apr. 25, 2016

Related U.S. Application Data
(60) Provisional application No. 62/323,475, filed on Apr. 15, 2016.
(60) Provisional application No. 62/308,697, filed on Mar. 15, 2016.
(60) Provisional application No. 62/244,991, filed on Oct. 22, 2015.
(60) Provisional application No. 62/152,707, filed on Apr. 24, 2015.

(51) Int. Cl. C07K 16/28; C07K 16/30

(52) U.S. Cl. CPC C07K 16/2863; C07K 16/3046; A61K 2039/507

(57) ABSTRACT

Provided herein are methods of treating an epidermal growth factor receptor (EGFR) extracellular domain (ECD) mutant cancer in a human patient by administering to the patient an oligoclonal anti-epidermal growth factor receptor (anti-EGFR) antibody combination (e.g., MM-151, comprising a first monoclonal antibody (P1X), a second monoclonal antibody (P2X), and a third monoclonal antibody (P3X), wherein P1X, P2X and P3X are in a 2:2:1 molar ratio). In one embodiment, the cancer comprises at least one mutation in the extracellular domain of EGFR selected from the group consisting of EGFR R451C, S464L, K467T, G465R, G465E, I491M, and S492R.

### CROSS-REFERENCE TO RELATED APPLICATIONS

This application claims priority to, and the benefit of, the following U.S. Provisional Applications: No. 62/152,707, filed on Apr. 24, 2015; No. 62/244,991, filed on Oct. 22, 2015; No. 62/308,697, filed on Mar. 15, 2016; and No. 62/323,475, filed on Apr. 15, 2016. The contents of the aforementioned applications are hereby incorporated herein by reference.

### BACKGROUND

The epidermal growth factor receptor (EGFR) is a cell surface transmembrane receptor of the HER/ErbB receptor family that transmits signals (including mitogenic signals that drive cell proliferation) to the interior of a cell when activated, typically by the binding of any of a number of extracellular ligands such as epidermal growth factor (EGF).

EGFR-targeted monoclonal antibodies such as cetuximab and panitumumab are not always effective against EGFR-expressing tumors. In many cases, patients that respond to and receive benefit from these antibody preparations end up developing a secondary resistance to the treatment. One cause of this resistance is the development of one or more mutations in the ectodomain of EGFR that render the tumor cells unresponsive to cetuximab and/or panitumumab.

One approach taken with the aim of improving anti-EGFR antibody efficacy has been to develop mixtures of anti-EGFR monoclonal antibodies (i.e., oligoclonal antibodies) targeted to different sites (epitopes) on to the extracellular domain of EGFR. See, e.g., PCT International Publication No. WO/2011/140254 and U.S. Pat. No. 7,887,805. These developments have created a need to enable the identification of cancer patients whose tumors have characteristics rendering them unresponsive to monoclonal anti-EGFR antibodies so that such patients may receive effective treatment via administration of oligoclonal anti-EGFR antibodies. The present disclosure answers this need and provides other benefits.

### SUMMARY

Provided herein are methods for treating and for selecting treatment for a cancer patient who has progressed on or become refractory to monoclonal anti-EGFR antibodies, e.g., cetuximab or panitumumab, the methods comprising determining whether a patient's tumor cells have acquired mutations in the EGFR gene region coding for the extracellular domain, wherein the selected treatment comprises an oligoclonal mixture of anti-EGFR antibodies that bind to the EGFR extracellular domain.

Also provided are methods of predicting whether a tumor (e.g., malignant tumor) will be responsive to treatment with oligoclonal anti-EGFR antibodies as described herein, but not responsive to treatment with single monoclonal anti-EGFR antibodies (e.g., cetuximab, panitumumab) or Sym004, the methods comprising determining whether a patient's tumor cells have acquired mutations in the EGFR gene region coding for the extracellular domain.

Further provided are methods of treating a patient having a tumor containing cells that harbor at least one mutation in an EGFR extracellular domain, the methods comprising administering to the patient an effective amount of an oligoclonal mixture of anti-EGFR antibodies that bind to the EGFR extracellular domain (e.g., in a single composition).

In one embodiment, the selected oligoclonal mixture of anti-EGFR antibodies comprises (a) a monoclonal antibody comprising heavy chain CDR1, CDR2, and CDR3 sequences of SEQ ID NOs: 1, 2, and 3 respectively, and light chain CDR1, CDR2, and CDR3 sequences of SEQ ID NOs: 4, 5, and 6, respectively; (b) a monoclonal antibody comprising heavy chain CDR1, CDR2, and CDR3 sequences of SEQ ID NOs: 7, 8, and 9, respectively, and light chain CDR1, CDR2, and CDR3 sequences of SEQ ID NOs: 10, 11 and 12, respectively; and (c) a monoclonal antibody comprising heavy chain CDR1, CDR2, and CDR3 sequences of SEQ ID NOs: 13, 14, and 15 respectively, and light chain CDR1, CDR2, and CDR3 sequences of SEQ ID NOs: 16, 17, and 18, respectively.

In another embodiment, the selected oligoclonal mixture of anti-EGFR antibodies comprises (a) a monoclonal antibody comprising a heavy chain variable region comprising SEQ ID NO: 19 and a light chain variable region comprising SEQ ID NO: 20; (b) a monoclonal antibody comprising a heavy chain variable region comprising SEQ ID NO: 21 and a light chain variable region comprising SEQ ID NO: 22; and (c) a monoclonal antibody comprising a heavy chain variable region comprising SEQ ID NO: 23 and a light chain variable region comprising SEQ ID NO: 24.

Other anti-EGFR oligoclonal antibody compositions which can be employed are described in PCT International Publication No. WO/2011/140254 and corresponding pending U.S. Patent Publication No. and corresponding pending U.S. Pat. No. 9,044,460, and in pending U.S. Pat. Nos. 9,226,964 and 7,887,805 (the “Oligoclonal Applications”), as well as oligoclonal mixtures of such antibodies in combination with other anti-EGFR antibodies, are useful for treatment of cancers, e.g., malignant (neoplastic) tumors.

In one embodiment, the selected oligoclonal antibodies (e.g., comprising (a), (b), and (c)) are combined (e.g., in a composition) at a molar ratio of 2:2:1 to each other.

In another embodiment, the patient is refractory to one or more of a monoclonal antibody therapeutic that binds to the EGFR extracellular domain, such as cetuximab, Sym004, or panitumumab. In another embodiment, the patient has had disease progression on or following a fluoropyrimidine-containing regimen, an oxaliplatin-containing regimen, or an irinotecan-containing regimen. In another embodiment the patient has progressed on or become refractory to an antineoplastic therapy after prior treatment with an antineoplastic agent. In another embodiment, the patient has become resistant to an antineoplastic therapy after prior treatment with an antineoplastic agent. In another embodiment, the patient is treated following disease progression or recurrence after prior treatment with an antineoplastic agent. In another embodiment, the patient is treated after failure of treatment with an antineoplastic agent. In another embodiment, the cancer is identified as a cancer that has acquired resistance to an antineoplastic agent.

In another embodiment, the patient has been diagnosed with colorectal cancer, metastatic colorectal cancer (e.g., KRAS, NRAS, and/or BRAF wild-type metastatic colorectal cancer), squamous cell head and neck cancer, recurrent or metastatic head and neck cancer, melanoma, breast cancer, ovarian cancer, renal carcinoma, gastrointestinal/colon cancer, lung cancer (e.g., NSCLC), or prostate cancer.

In still other embodiments, the tumor tested and/or treated according to the methods of the invention is a tumor of the skin, central nervous system, head, neck, esophagus, stomach, colon, rectum, anus, liver, pancreas, bile duct, gallbladder, lung, breast, ovary, uterus, cervix, vagina, testis, germ cells, prostate, kidney, ureter, urinary bladder, adrenal, pituitary, thyroid, bone, muscle or connective tissue.

In one embodiment, the tumor is determined to comprise cells that harbor at least one mutation in an EGFR extracellular domain based on an FDA-approved test. In another embodiment, the tumor contains cells harboring a mutation in Domain III of the EGFR extracellular domain. In another embodiment, the tumor contains cells harboring a mutation in exon 12 of the EGFR gene.

In certain embodiments, the methods of the invention comprise obtaining a biopsy sample of the tumor, and determining whether the tumor has at least one mutation in the DNA or RNA coding for the extracellular domain of EGFR using polymerase chain reaction or next-generation sequencing (NGS). In another embodiment, the method comprises obtaining a biopsy sample of the tumor, and determining whether the tumor has at least one mutation in the extracellular domain of EGFR using immunohistochemistry or mass spectrometry. In another embodiment, the method comprises obtaining a blood sample from the patient, and determining whether the blood sample contains circulating DNA with one or more mutations in sequences encoding the extracellular domain of EGFR.

In particular embodiments, the circulating DNA is cell-free DNA isolated from the blood sample or is isolated from circulating tumor cells in the blood sample. In other embodiments, the circulating DNA is DNA or RNA isolated from circulating exosomes in the blood sample. In other embodiments, the circulating DNA is cell-free DNA isolated from a urine sample. In other embodiments, the biopsied tumor cells, circulating tumor cells, or cell free DNA harbor at least one mutation in DNA or RNA coding for the extracellular domain of EGFR, and the at least one mutation is in exon 12 of the EGFR gene.

Another embodiment, the mutation in the extracellular domain of EGFR is a protein sequence change, or a DNA or RNA coding region that results in a protein sequence change, chosen from the group consisting essentially of EGFR R451C, S464L, K467T, G465R, G465E, I491M, and S492R. In particular embodiments, the protein sequence change is S492R.

In another embodiment, the treatment methods described herein comprise administering the anti-EGFR antibodies in combination with one or more other antineoplastic agents (e.g., other chemotherapeutics or other small molecule drugs). In one embodiment, no more than three other antineoplastic agents are administered within a treatment cycle. In another embodiment, no more than two other antineoplastic agents are administered within a treatment cycle. In another embodiment, no more than one other antineoplastic agent is administered within a treatment cycle. In another embodiment, no other antineoplastic agent is administered within a treatment cycle.

Also provided are methods of treating an epidermal growth factor receptor (EGFR) extracellular domain (ECD) mutant cancer in a human patient, the method comprising administering to the patient an oligoclonal anti-epidermal growth factor receptor (anti-EGFR) antibody combination. In one embodiment, the first monoclonal antibody is P1X, the second monoclonal antibody is P2X, the third monoclonal antibody is P3X, and the oligoclonal antibody combination comprises P1X, P2X and P3X in a 2:2:1 molar ratio. In another embodiment, the first monoclonal antibody comprises heavy chain CDR1, CDR2, and CDR3 sequences of SEQ ID NOs: 1, 2, and 3 respectively, and light chain CDR1, CDR2, and CDR3 sequences of SEQ ID NOs: 4, 5, and 6, respectively; the second monoclonal antibody comprises heavy chain CDR1, CDR2, and CDR3 sequences of SEQ ID NOs: 7, 8, and 9, respectively, and light chain CDR1, CDR2, and CDR3 sequences of SEQ ID NOs: 10, 11 and 12, respectively; and the third monoclonal antibody comprises heavy chain CDR1, CDR2, and CDR3 sequences of SEQ ID NOs: 13, 14, and 15 respectively, and light chain CDR1, CDR2, and CDR3 sequences of SEQ ID NOs: 16, 17, and 18, respectively. In another embodiment, the first monoclonal antibody comprises heavy chain CDR1, CDR2, and CDR3 sequences of SEQ ID NOs: 1, 2, and 3 respectively, and light chain CDR1, CDR2, and CDR3 sequences of SEQ ID NOs: 4, 5, and 6, respectively; the second monoclonal antibody comprises heavy chain CDR1, CDR2, and CDR3 sequences of SEQ ID NOs: 7, 8, and 9, respectively, and light chain CDR1, CDR2, and CDR3 sequences of SEQ ID NOs: 10, 11 and 12, respectively; and the third monoclonal antibody comprises heavy chain CDR1, CDR2, and CDR3 sequences of SEQ ID NOs: 13, 14, and 15 respectively, and light chain CDR1, CDR2, and CDR3 sequences of SEQ ID NOs: 16, 17, and 18, respectively. In one embodiment, the methods include treating an EGFR extracellular domain (ECD) mutant cancer in a human patient following treatment of the cancer with a different anti-EGFR therapy (e.g., cetuximab, panitumumab or Sym004). In one embodiment, the oligoclonal antibody combination is administered to the patient after obtaining a blood sample from the patient and determining that the blood sample contains circulating DNA with one or more mutations in sequences encoding the extracellular domain of EGFR. In one embodiment, the oligoclonal antibody combination is administered to the patient following detection of a mutation in Domain III or Domain IV of the EGFR extracellular domain in the blood of the patient. In another embodiment, the oligoclonal antibody combination is administered to the patient following detection of an exon 12 mutation in the ECD region of EGFR in the human patient.

In one embodiment, the cancer comprises at least one mutation in the extracellular domain of EGFR, which is a protein sequence change, or a DNA or RNA coding region that results in a protein sequence change, selected from the group consisting of EGFR R451C, S464L, K467T, G465R, G465E, I491M, and S492R. In another embodiment, the mutation in the extracellular domain of EGFR is arginine to cysteine at position 451 of SEQ ID NO: 27 (i.e., amino acid sequence corresponding to the extracellular domain of EGFR). In another embodiment, the mutation in the extracellular domain of EGFR is serine to leucine at position 464 of SEQ ID NO: 27. In another embodiment, the mutation in the extracellular domain of EGFR is lysine to threonine at position 467 of SEQ ID NO: 27. In another embodiment, the mutation in the extracellular domain of EGFR is glycine to arginine at position 465 of SEQ ID NO: 27. In another embodiment, the mutation in the extracellular domain of EGFR is glycine to glutamate at position 465 of SEQ ID NO: 27. In another embodiment, the mutation in the extracellular domain of EGFR is isoleucine to methionine at position 491 of SEQ ID NO: 27. In another embodiment, the mutation in the extracellular domain of EGFR is serine to arginine at position 492 of SEQ ID NO: 27.

In one embodiment, the method of treatment involves treatment of an EGFR extracellular domain (ECD) mutant cancer, such as a K-Ras, N-Ras, and/or BRAF wild-type metastatic colorectal cancer. In one embodiment, the mutant cancer is K-Ras wild-type, EGFR-expressing metastatic colorectal cancer as determined by an FDA-approved test. In another embodiment, the mutant cancer is a head and neck cancer (e.g., locally or regionally advanced squamous cell carcinoma of the head and neck). In another embodiment, the mutant cancer is Ras-wild type KRAS (exon 2) metastatic colorectal cancer (mCRC) as determined by an FDA-approved test.

In another aspect, the methods include treating a human patient having an epidermal growth factor receptor (EGFR) extracellular domain (ECD) mutant cancer having at least one detected mutation in the ECD of EGFR selected from the group consisting of EGFR R451C, S464L, K467T, G465R, G465E, I491M, and S492R, the method comprising administering to the patient an oligoclonal anti-epidermal growth factor receptor (anti-EGFR) antibody combination comprising three monoclonal antibodies, wherein the first monoclonal antibody is P1X, the second monoclonal antibody is P2X, the third monoclonal antibody is P3X, and the oligoclonal antibody combination comprises P1X, P2X and P3X in a 2:2:1 molar ratio.

In a another aspect, the methods include treating a human patient having an epidermal growth factor receptor (EGFR) extracellular domain (ECD) mutant cancer having a detected mutation in the Domain III or Domain IV of the ECD of EGFR, the method comprising administering to the patient a therapeutically effective amount of a MM-151 oligoclonal anti-epidermal growth factor receptor (anti-EGFR) antibody combination, wherein the oligoclonal antibody combination consists of a P1X monoclonal antibody, a P2X monoclonal antibody, and a P3X monoclonal antibody in a 2:2:1 molar ratio.

Also provided are uses of an oligoclonal anti-epidermal growth factor receptor (anti-EGFR) antibody combination (e.g., MM-151) in the treatment of an EGFR extracellular domain (ECD) mutant cancer in a human patient. In one embodiment, the first monoclonal antibody is P1X, the second monoclonal antibody is P2X, the third monoclonal antibody is P3X, and the oligoclonal antibody combination comprises P1X, P2X and P3X in a 2:2:1 molar ratio. In another embodiment, the first monoclonal antibody comprises heavy chain CDR1, CDR2, and CDR3 sequences of SEQ ID NOs: 1, 2, and 3 respectively, and light chain CDR1, CDR2, and CDR3 sequences of SEQ ID NOs: 4, 5, and 6, respectively; the second monoclonal antibody comprises heavy chain CDR1, CDR2, and CDR3 sequences of SEQ ID NOs: 7, 8, and 9, respectively, and light chain CDR1, CDR2, and CDR3 sequences of SEQ ID NOs: 10, 11 and 12, respectively; and the third monoclonal antibody comprises heavy chain CDR1, CDR2, and CDR3 sequences of SEQ ID NOs: 13, 14, and 15 respectively, and light chain CDR1, CDR2, and CDR3 sequences of SEQ ID NOs: 16, 17, and 18, respectively. In another embodiment, the first monoclonal antibody comprises heavy chain CDR1, CDR2, and CDR3 sequences of SEQ ID NOs: 1, 2, and 3 respectively, and light chain CDR1, CDR2, and CDR3 sequences of SEQ ID NOs: 4, 5, and 6, respectively; the second monoclonal antibody comprises heavy chain CDR1, CDR2, and CDR3 sequences of SEQ ID NOs: 7, 8, and 9, respectively, and light chain CDR1, CDR2, and CDR3 sequences of SEQ ID NOs: 10, 11 and 12, respectively; and the third monoclonal antibody comprises heavy chain CDR1, CDR2, and CDR3 sequences of SEQ ID NOs: 13, 14, and 15 respectively, and light chain CDR1, CDR2, and CDR3 sequences of SEQ ID NOs: 16, 17, and 18, respectively.

In one embodiment, the use of the oligoclonal anti-epidermal growth factor receptor (anti-EGFR) antibody combination (e.g., MM-151) in the treatment of an EGFR extracellular domain (ECD) mutant cancer in a human patient follows treatment of the cancer with a different anti-EGFR therapy (e.g., cetuximab, panitumumab or Sym004). In one embodiment, the use follows detection of a mutation in Domain III or Domain IV of the EGFR extracellular domain in the human patient. In another embodiment, the use follows detection of an exon 12 mutation in the ECD region of EGFR in the human patient. In another embodiment, there is at least one mutation in the extracellular domain of EGFR, which is a protein sequence change, or a DNA or RNA coding region that results in a protein sequence change, selected from the group consisting of EGFR R451C, S464L, K467T, G465R, G465E, I491M, and S492R. In one embodiment, the mutation is detected after obtaining a blood sample from the patient and determining that the blood sample contains circulating DNA with one or more mutations in sequences encoding the extracellular domain of EGFR.

In one embodiment, the use involves treatment of an EGFR extracellular domain (ECD) mutant cancer, such as a K-Ras, N-Ras, and/or BRAF wild-type metastatic colorectal cancer. In one embodiment, the mutant cancer is Ras wild-type, EGFR-expressing metastatic colorectal cancer as determined by an FDA-approved test. In another embodiment, the mutant cancer is a head and neck cancer (e.g., locally or regionally advanced squamous cell carcinoma of the head and neck). In another embodiment, the mutant cancer is Ras-wild type metastatic colorectal cancer (mCRC) as determined by an FDA-approved test.

Further provided are uses of an oligoclonal anti-epidermal growth factor receptor (anti-EGFR) antibody combination in the treatment of an epidermal growth factor receptor (EGFR) extracellular domain (ECD) mutant cancer having at least one detected mutation in the ECD of EGFR selected from the group consisting of EGFR R451C, S464L, K467T, G465R, G465E, I491M, and S492R, wherein the oligoclonal anti-epidermal growth factor receptor (anti-EGFR) antibody combination comprises three monoclonal antibodies, wherein the first monoclonal antibody is P1X, the second monoclonal antibody is P2X, the third monoclonal antibody is P3X, and the oligoclonal antibody combination comprises P1X, P2X and P3X in a 2:2:1 molar ratio.

### BRIEF DESCRIPTION OF THE FIGURES

FIGS. 1A-1G are seven graphs showing the interaction between EGF ligand and EGFR mutants. HEK 293 cells were transfected with plasmids expressing the indicated NanoLuc®-EGFR mutants and then treated with increasing dose of EGF tracer (HaloTag®-EGF) in the presence (light grey line with circle markers) or absence (black line with triangle markers) of an excess amount of unabeled EGF to assess that EGF tracer can effectively bind to the NanoLuc-EGFR, including EGFR wild-type (FIG. 1A), EGFR R451C (FIG. 1B), S464L (FIG. 1C), K467T (FIG. 1D), G465R (FIG. 1E), I491M (FIG. 1F), and S492R (FIG. 1G). An increasing dose of unlabeled EGF (dark grey line with square markers) is used as an assay control to demonstrate that the measured signal corresponds to the HaloTag®-EGF.

FIGS. 2A-2G are seven graphs showing that MM-151 is able to bind all EGFR ectodomain mutants in a drug displacement assay. HEK 293 cells were transfected with plasmids expressing wild-type EGFR (FIG. 2A) and the NanoLuc-EGFR mutants EGFR R451C (FIG. 2B), S464L (FIG. 2C), K467T (FIG. 2D), G465R (FIG. 2E), I491M (FIG. 2F), and S492R (FIG. 2G). The mutants were then treated with increasing doses (from 0 to 100 μg/ml) of the anti-EGFR drugs cetuximab (“CMAB”, grey triangles), panitumumab (“PMAB”, light grey circles) or MM-151 (black squares) and HaloTag-EGF tracer (at a concentration of 18 ng/mL).

FIGS. 3A-3H are eight graphs showing that LIM1215 cells (human colorectal carcinoma) that overexpress EGFR ectodomain mutants, and are resistant to cetuximab or both to cetuximab and panitumumab, are sensitive to MM-151. LIM1215 were infected with lentivirus expressing the indicated EGFR ectodomain mutants and selected cells were treated for 6 days with increasing concentrations of cetuximab (“CMAB”; dark grey line with triangle markers), panitumumab (“PMAB”; light grey line with circle markers) and MM-151 (black line with square markers). Cell viability was measured by the adenosine triphosphate (ATP) assay. Mutants and controls shown are GFP control (FIG. 3A), EGFR wild-type (FIG. 3B), EGFR R451C (FIG. 3C), S464L (FIG. 3D), K467T (FIG. 3E), G465R (FIG. 3F), I491M (FIG. 3G), and S492R (FIG. 3H).

FIGS. 4A-4H are a series of images of western blot analysis of EGFR signaling in LIM1215 cells that overexpress EGFR ectodomain mutants. LIM1215 cells were infected with lentivirus expressing the indicated EGFR ectodomain mutants. Mutants and controls shown are GFP control (FIG. 4A), EGFR wild-type (FIG. 4B), EGFR R451C (FIG. 4C), S464L (FIG. 4D), K467T (FIG. 4E), G465R (FIG. 4F), I491M (FIG. 4G), and S492R (FIG. 4H). Selected cells were cultured in the presence of cetuximab (“CETUX”), panitumumab (“PMAB”) or MM-151 for 2 hours and then stimulated with EGF (5 ng/ml) for 15 minutes. Western blot analysis was performed on cell lysates using antibodies specific to the indicated proteins including phosphorylated EGFR (“pEGFR”), total cellular EGFR (“TOT EGFR”), phosphorylated AKT (“pAKT”), total cellular AKT (“TOT AKT”), phosphorylated ERK (“pERK”), total cellular ERK (“TOT ERK”) and vinculin (a housekeeping control to demonstrate similar protein concentration across samples within the panel).

FIGS. 5A-5E are five graphs showing that LIM1215 cells (human colorectal carcinoma) that harbor acquired resistance to cetuximab through expression of EGFR ectodomain mutations are sensitive to MM-151. Acquired resistance was generated in vitro through continuous exposure to 1.4 μM cetuximab over repeated cell passages for 3-9 months. Sub-cloning was then performed to isolate sub-populations that have acquired a mutation to the EGFR extracellular domain. Cells were then cultured in the presence of cetuximab (“CETUX”), panitumumab (“PMAB”) or MM-151 for 6 days. Cell viability was measured by the adenosine triphosphate (ATP) assay. The controls for this assay are the parental cell line (“LIM1215 WT”; FIG. 5A) and a cell line engineered to express the S492R mutant (“LIM1215 KI EGFR S492R”; FIG. 5E). Acquired resistant cell lines shown are the “R5” population (FIG. 5B) and a sub-cloned cell line that was identified as harboring an EGFR G465R mutation (FIG. 5D). Also shown is a sub-cloned cell line from the “R1” population that was identified as harboring an EGFR I491M mutation (FIG. 5C).

FIGS. 6A-6C are three graphs showing that LIM1215 cells (human colorectal carcinoma) that harbor acquired resistance to cetuximab through expression of EGFR ectodomain mutations are sensitive to MM-151. Acquired resistance was generated in vitro through continuous exposure to 1.4 μM cetuximab over repeated cell passages for 3-9 months. Sub-cloning was then performed to isolate sub-populations that have acquired a mutation to the EGFR extracellular domain. Cells were then cultured in the presence of cetuximab (“CETUX”), panitumumab (“PMAB”) or MM-151 for 6 days. Cell viability was measured by the adenosine triphosphate (ATP) assay. The control for this assay is the parental cell line (“OXCO2 WT”; FIG. 6A). Acquired resistant cell lines shown are the “R2” population (FIG. 6B) and a sub-cloned cell line that was identified as harboring co-expression of EGFR I491M and NRAS G13D mutations (“OXCO2 R2 cl.88”; FIG. 6C).

FIGS. 7A-7D are four graphs showing that CCK81 and HCA46 cells (human colorectal carcinoma) that harbor acquired resistance to cetuximab through expression of EGFR ectodomain mutations are sensitive to MM-151. Acquired resistance was generated in vitro through continuous exposure to 1.4 μM cetuximab over repeated cell passages for 3-9 months (HCA46) or stepwise from 680 nM to 1.4 μM cetuximab for 6 months (CCK81). Sub-cloning was then performed to isolate sub-populations that have acquired a mutation to the EGFR extracellular domain. Cells were then cultured in the presence of cetuximab (“CETUX”), panitumumab (“PMAB”) or MM-151 for 6 days. Cell viability was measured by the adenosine triphosphate (ATP) assay. The controls for this assay are the respective parental cell lines “CCK81 WT” (FIG. 7A) and “HCA46 WT” (FIG. 7C). Shown are acquired resistance sub-clone cell lines of CCK81 (FIG. 7B) and HCA46 (FIG. 7D), harboring EGFR S464L and EGFR G465E mutations, respectively.

FIGS. 8A-8D are a series of images of western blot analysis of EGFR signaling in LIM1215 (FIG. 8A), OXCO2 (FIG. 8B), CCK81 (FIG. 8C), and HCA46 (FIG. 8D) cells that harbor acquired resistance to cetuximab through expression of EGFR ectodomain mutations. Acquired resistance was generated in vitro through continuous exposure to 1.4 μM cetuximab over repeated cell passages for 3-9 months (LIM1215, OXCO2, and HCA46) or stepwise from 680 nM to 1.4 μM cetuximab for 6 months (CCK81). Sub-cloning was then performed to isolate sub-populations that have acquired a mutation to the EGFR extracellular domain. Selected cells were cultured in the presence of cetuximab (“CETUX”), panitumumab (“PMAB”) or MM-151 for 2 hours and then stimulated with EGF (10 ng/ml) for 15 minutes. Western blot analysis was performed on cell lysates using antibodies specific to the indicated proteins including phosphorylated EGFR (“pEGFR”), total cellular EGFR (“TOT EGFR”), phosphorylated AKT (“pAKT”), total cellular AKT (“TOT AKT”), phosphorylated ERK (“pERK”), total cellular ERK (“TOT ERK”) and vinculin (a housekeeping control to demonstrate similar protein concentration across samples within the panel). Shown for the LIM1215 cell line are parental cell line (“LIM1215 WT”), the “R5” acquired resistance population, the “CL55” sub-clone of the “R5” population that harbors the EGFR G565R mutation, the “CLONE4” sub-clone of the “R1” population that harbors co-expression of EGFR I491M and NRAS G12C mutations, and the “LIM1215 KI” cell line was engineered to express the S492R mutation. Shown for the OXCO2 cell line are the parental cell line (“OXCO2 WT”), the “R2” acquired resistance population, the “CL88” sub-clone of the “R2” population that harbors co-expression of EGFR I491M and NRAS G13D mutations, and the “CL113” sub-clone of the “R2” population that harbors expression of an NRAS G13D mutation. Shown for CCK81 cell line are the parental cell line (“CCK81 WT”) and a sub-clone of the “R1” acquired resistance population that harbors expression of the EGFR S464L mutation. Shown for HCA46 cell line are the parental cell line (“HCA46 WT”) and a sub-clone of the “R5” population that harbors expression of the EGFR G465E mutation.

FIGS. 9A-9C are a series of three graphs showing that a cell line derived from a clinical colorectal tumor sample (“xenopatient”) harboring a G465E mutation in the EGFR ectodomain is sensitive to MM-151. Sub-cloning was performed to isolate sub-populations that also harbor the G465E mutation. Cells were then cultured in the presence of cetuximab (“CETUX”), panitumumab (“PMAB”) or MM-151 for 6 days. Cell viability was measured by the adenosine triphosphate (ATP) assay. Shown are the xenopatient-derived cell line (FIG. 9A) and the two sub-cloned cell lines (FIG. 9B and FIG. 9C).

FIGS. 10A-10C are a series of three graphs showing the ability of individual antibodies within three antibody combinations to simultaneously engage the same EGFR molecule. A bio-layer interferometry assay (ForteBio®) was performed with one antibody within the combination conjugated to the biosensor followed by incubations with recombinant EGFR extracellular domain mutant and then the remaining antibody(ies) within the combination. The order of incubation is as indicated in each figure panel. Assessed are the EGFR extracellular domain mutants K467T, R451C, and S492R. The antibody combinations assessed are MM-151 (FIG. 10A), a combination of three antibodies (25E+P2X+P3X; FIG. 10B), and a combination of two antibodies (Sym992 and Sym1024; FIG. 10C).

FIGS. 11A-11B are a series of two graphs showing that LIM1215 cells (human colorectal carcinoma) that overexpress the EGFR S492R ectodomain mutation and are activated by a representative EGFR ligand are sensitive to MM-151. LIM1215 were stably transfected to express the S492R EGFR ectodomain mutant and selected cells were treated for 3 days with increasing concentrations of cetuximab (“CMAB”; dark grey line with triangle markers), a combination of Sym992 and Sym1024 antibodies (“Sym004”; light grey line with circle markers) and MM-151 (black line with square markers) in the presence of 8 nM of recombinant human EGF ligand. Cell viability was measured by the adenosine triphosphate (ATP) assay. Shown are the parental LIM1215 cell line (untransfected; expresses only wild type EGFR)(FIG. 11A) and the transfected LIM1215 cell line that overexpresses the EGFR S492R mutation (FIG. 11B).

FIGS. 12A-12C are a series of three graphs showing that LIM1215 cells (human colorectal carcinoma) that either overexpress the EGFR S492R ectodomain mutation or are engineered to express the EGFR S492R ectodomain mutation are sensitive to MM-151 in an in vivo murine xenograft model. Female nu/nu mice (NU-Foxn1^{nu}; Charles River Labs), aged 4-5 weeks, weighing 16±0.5 g, were inoculated with 0.2 mL of LIM1215 (#2), LIM1215 (#2) EGFR S492R, or LIM1215 (#2) EGFR S492R KI cell suspension in PBS containing growth factor-reduced Matrigel (BD Biosciences) at a density of 5×10⁶ cells per mouse. Once tumors had reached approximately 300 mm³ in volume, mice were randomized into treatment groups (10 mice/group) to receive PBS, cetuximab at 12.5 or 25 mg/kg, or MM-151 tool compound (consisting of 25E, P2X, and P3X antibodies). Treatments were administered by IP injection, with cetuximab, 25E, and P3X on a Q7D schedule (e.g. Monday) and P2X on 3× weekly schedule (e.g. Monday, Wednesday, Friday). The MM-151 tool compound was administered at doses calculated to result in summed exposure for the combination equivalent to the cetuximab dose levels of 12.5 mg/kg (6.25 mg/kg Q7D of 25E; 8.75 mg/kg 3× weekly of P2X; 3.125 mg/kg Q7D of P3X) and 25 mg/kg (12.5 mg/kg Q7D of 25E; 17.5 mg/kg 3× weekly of P2X6.25 mg/kg Q7D of P3X). Cetuximab, 25E, and P3X were administered a 2× loading dose on the first administration followed by maintenance doses at the indicated dose levels on subsequent administrations. Tumor dimensions were measured three times each week by calipers and the tumor volumes were calculated using the formula: π/6×L×W², where L and W, respectively, represent the larger and smaller tumor diameter.

FIGS. 13A-13B are a series of two graphs that shows an overlay of the clinical response and the presence of EGFR ECD mutations in two human colorectal cancer patient treated with MM-151 in an early phase clinical trial (NCT #01520389). Serum samples were drawn before the administration of MM-51 on the indicated dates and the presence of EGFR ECD mutations detected by droplet digital PCR (ddPCR) with mutation-specific primers. The allelic frequency of each EGFR ECD mutation is reflective of the percent of cell-free DNA (cfDNA) in the serum sample that harbors the indicated mutation. Clinical response is defined by Response Evaluation Criteria in Solid Tumors (RECIST) 1.1 guidelines and was measured at the indicated visits (including baseline before treatment with MM-151, during the course of treatment with MM-151, and upon disease progression). The first patient (095) was screened for enrollment on the clinical trial on Aug. 29, 2014, first treated on Sep. 23, 2014, and treated until disease progression was assessed on Jan. 22, 2015. The second patient (051) was screened for enrollment on the clinical trial on Feb. 25, 2013, first treated on Mar. 19, 2013, and treated until disease progression was assessed on Aug. 28, 2013.

FIG. 14 is a graph showing the ability of individual antibodies to engage EGFR on LIM1215 cells (human colorectal carcinoma) that express wild type EGFR (LIM1215 (#2)) or are engineered to express the EGFR S492R ectodomain mutation (LIM1215 (#2) EGFR S492R KI). Antibodies were labeled with Alexa Fluor® 488 dye and binding to EGFR on the surface of cells measured by flow cytometry. The antibodies assessed are cetuximab (“CMAB”), P1X, P2X, and P3X. Results for each individual antibody are normalized to the binding value observed on the LIM1215 (#2) control cell line.

FIGS. 15A-15C are a series of three graphs showing that LIM1215 cells (human colorectal carcinoma) that either overexpress the EGFR S492R ectodomain mutation or are engineered to express the EGFR S492R ectodomain mutation and are activated by a representative EGFR ligand are sensitive to MM-151. LIM1215 (#2) cells were engineered via the CRISPR/Cas9 targeted genome editing system to express the S492R EGFR ectodomain mutant. Cells were treated for 3 days with cetuximab (“CMAB”), panitumumab (“PMAB”), a combination of Sym992 and Sym1024 antibodies (“Sym004”) and MM-151 in the presence of 8 nM of recombinant human EGF ligand. Cell viability was measured by the adenosine triphosphate (ATP) assay. Shown in FIG. 15A are the LIM1215 (#2) cell line (expresses only wild type EGFR), the engineered LIM1215 (#2) EGFR S492R KI cell line that expresses the EGFR S492R mutation, and the transfected LIM1215 (#2) EGFR S492R cell line that overexpresses the EGFR S492R mutation, each treated with a 1 μM concentration of each inhibitor. Shown in FIGS. 15B and 15C are the parental LIM1215 (#2) cell line and the engineered LIM1215 (#2) EGFR S492R KI cell line that expresses the EGFR S492R mutation, respectively, each treated with increasing concentrations of cetuximab (dark grey line with triangle markers), panitumumab (light gray line with diamond markers), Sym004 (gray line with circle markers), or MM-151 (black line with square markers).

FIG. 16A-16B are graphs showing that LIM1215 cells (human colorectal carcinoma) that are engineered to express the EGFR S492R or G465R ectodomain mutations expand over the course of treatment with cetuximab but are sensitive to MM-151. Two LIM1215 cell line pools are used—one contains a mixture of cells that express EGFR wild type and EGFR S492R mutant and a second that contains cells that express EGFR wild type and EGFR G465R. These pools were generated during the process of engineering the LIM1215 (#2) EGFR S492R KI and LIM1215 (#2) EGFR G465R KI cell lines. The initial allelic frequency on Day 0 in the EGFR S492R mutant pool was measured to be 4.6% (mutant EGFR S492R to wild type EGFR) and the initial allelic frequency on Day 0 in the EGFR G465R mutant pool was 0.6%. Cells were treated over fifteen (S492R) or seven (G465R) days with cetuximab (“CMAB”, dark gray line with triangle markers), MM-151 (black line with square markers) or media alone (light gray line with circle markers) and the allelic frequency of the EGFR ectodomain mutations measured in genomic DNA collected on days 4, 10, and 15 (S492R) or on days 0, 3, and 7 (G465R). As a negative control, the experiment was also performed with an undiluted plate of LIM1215 (#2) cells that expresses wild type EGFR (stippled lines) and an allelic frequency of 0% measured at all time points for all treatment conditions.

FIGS. 17A-17B are a schematic of the human EGFR gene and a table of EGFR ectodomain mutations. Shown in FIG. 17A is a schematic of the human EGFR gene with markings to note the seven structural protein domains, the 28 DNA coding exons, and the amino acid sequence numbers that mark the boundaries between each protein domain. Not shown is a putative signal peptide that includes amino acids 1-24. There are four extracellular domains (I, II, III, IV) that together comprise the following 621 amino acids and the first 15 exons (exon #15 spans extracellular domain IV and the transmembrane domain). EGFR ectodomain mutations have been identified in exon 12 which encodes amino acids 433-499. This region spans the end portion of extracellular domain III and the beginning portion of domain IV. Shown in FIG. 17B is a table that lists EGFR ectodomain mutations that have been identified in clinical and/or preclinical samples and published in literature. Mutations are identified by the amino acid change, the DNA base change, and the DNA codon change.

FIG. 18 is a series of images of western blot analysis of EGFR signaling showing that LIM1215 cells (human colorectal carcinoma) that either overexpress the EGFR S492R ectodomain mutation or are engineered to express the EGFR S492R ectodomain mutation and are activated by a representative EGFR ligand are sensitive to MM-151. Shown are LIM1215 (#2), LIM1215 (#2) EGFR S492R KI, and LIM1215 (#2) EGFR S492R cells that harbor expression of wild type EGFR, engineered expression of the EGFR S492R ectodomain mutation, or over-expression of the EGFR S492R ectodomain mutation, respectively. Cells were cultured in the presence of 100 nM of cetuximab, Sym004, or MM-151 for 24 hours and then stimulated with AREG (8 nM) or EGF (8 nM) ligand for 10 minutes. As controls, cells were left untreated (no drug or ligand) or with each ligand alone, as indicated. Western blot analysis was performed on cell lysates using antibodies specific to the indicated proteins including phosphorylated EGFR (“pEGFR”) and actin (a housekeeping control to demonstrate similar protein concentration across samples within the panel).

FIGS. 19A-19C are a series of three plots of ELISA analysis of ERK signaling showing that LIM1215 cells (human colorectal carcinoma) that either overexpress the EGFR S492R ectodomain mutation or are engineered to express the EGFR S492R ectodomain mutation and are activated by a representative EGFR ligand are sensitive to MM-151. Shown are LIM1215 (#2), LIM1215 (#2) EGFR S492R KI, and LIM1215 (#2) EGFR S492R cells that harbor expression of wild type EGFR, engineered expression of the EGFR S492R ectodomain mutation, or over-expression of the EGFR S492R ectodomain mutation, respectively. Cells were treated with increasing concentrations (1:4 serial dilutions between 1000 and 0.244 nM) with cetuximab, Sym004, or MM-151 for two hours and then stimulated with AREG (8 nM) or EGF (8 nM) ligand for 10 minutes. Phosphorylated ERK protein was measured by ELISA. Results were normalized by ligand (EGF or AREG) to the median value of the control samples with ligand stimulation alone (no drug). Shown in FIG. 19A is a heatmap representation of the normalized phospho-ERK response following treatment with increasing concentrations of drug and stimulation with EGF ligand. Shown in FIGS. 19B and 19C are bar plots of the normalized phospho-ERK response following treatment with 250 nM of drug and stimulation with EGF or AREG, respectively.

FIGS. 20A-20I are a series of nine plots of cell growth rate analysis showing that LIM1215 cells (human colorectal carcinoma) that either overexpress the EGFR S492R ectodomain mutation or are engineered to express the EGFR S492R ectodomain mutation and are activated by a representative EGFR ligand are sensitive to MM-151. Shown are LIM1215 (#2), LIM1215 (#2) EGFR S492R KI, and LIM1215 (#2) EGFR S492R cells that harbor expression of wild type EGFR, engineered expression of the EGFR S492R ectodomain mutation, or over-expression of the EGFR S492R ectodomain mutation, respectively. Cells were treated with increasing concentrations (between 0.15 and 1000 nM) of cetuximab, panitumumab, Sym004, or MM-151 in the presence of no ligand, AREG (8 nM) or EGF (8 nM). Cell confluency was measured at two hour intervals over a period of 72 hours in a live-cell imager. The growth rates were calculated for each treatment condition and results normalized to control samples (no drug) for each cell line.

### DETAILED DESCRIPTION

The terms “EGFR,” and “EGF receptor” are used interchangeably herein to refer to human EGFR protein (also referred to as ErbB1 or HER1); see UniProtKB/Swiss-Prot entry P00533. The EGFR extracellular domain, or EGFR-ECD, is the portion of the EGFR protein that extends beyond the cell surface in vivo, and is thus accessible to antibodies on the exterior of the cell. The wild-type EGFR-ECD protein sequence is set forth in SEQ ID NO:19. As used herein, an “EGFR-ECD mutation” or a “mutation in the extracellular domain of EGFR” may refer to an EGFR-ECD protein sequence with a difference in at least one amino acid residue as compared to the wild type sequence; an “EGFR-ECD mutation” may also refer to a change in that portion of the DNA or RNA coding sequence that corresponds to a change in the protein sequence of the extracellular domain of EGFR. In some embodiments, the change in the DNA or RNA coding sequence occurs in exon 12 of the EGFR gene or transcript. In other embodiments, the EGFR-ECD mutation is a change in the protein sequence corresponding to Domain III of the extracellular domain of EGFR.

The term “antibody” describes polypeptides comprising at least one antibody derived antigen binding site (e.g., VH/VL region or Fv, or CDR). Antibodies include known forms of antibodies. For example, the antibody can be a human antibody, a humanized antibody, a bispecific antibody, or a chimeric antibody. The antibody also can be a Fab, Fab′2, ScFv, SMIP, Affibody®, nanobody, or a domain antibody. The antibody also can be of any of the following isotypes: IgG1, IgG2, IgG3, IgG4, IgM, IgA1, IgA2, IgAsec, IgD, and IgE. The antibody may be a naturally occurring antibody or may be an antibody that has been altered by a protein engineering technique (e.g., by mutation, deletion, substitution, conjugation to a non-antibody moiety). For example, an antibody may include one or more variant amino acids (compared to a naturally occurring antibody) which changes a property (e.g., a functional property) of the antibody. For example, numerous such alterations are known in the art which affect, e.g., half-life, effector function, and/or immune responses to the antibody in a patient. The term antibody also includes artificial or engineered polypeptide constructs which comprise at least one antibody-derived antigen binding site.

The term “monoclonal antibody” as used herein refers to an antibody obtained from a population of substantially homogeneous antibodies, i.e., the individual antibodies comprising the population are identical except for possible naturally occurring mutations that may be present in minor amounts. The term “oligoclonal antibody mixture” or “antibody mixture” refers to a combination of two or more antibodies, e.g., monoclonal antibodies, which are present in a single composition. In particular embodiments, the oligoclonal antibody mixture or antibody mixture is MM-151. Another example of an antibody mixture is Sym004 (Symphogen).

Antibodies, or antigen-binding fragments thereof described herein, used in the methods described herein can be generated using a variety of art-recognized techniques. Monoclonal antibodies may be obtained by various techniques familiar to those skilled in the art. Briefly, spleen cells from an animal immunized with a desired antigen are immortalized, commonly by fusion with a myeloma cell (see, Kohler & Milstein, Eur. J. Imanol. 6: 511-519 (1976)). Alternative methods of immortalization include transformation with Epstein Barr Virus, oncogenes, or retroviruses, or other methods well known in the art. Colonies arising from single immortalized cells are screened for production of antibodies of the desired specificity and affinity for the antigen, and yield of the monoclonal antibodies produced by such cells may be enhanced by various techniques, including injection into the peritoneal cavity of a vertebrate host. Alternatively, one may isolate DNA sequences which encode a monoclonal antibody or a binding fragment thereof by screening a DNA library from human B cells according to the general protocol outlined by Huse, et al., Science 246: 1275-1281 (1989).

The term “inhibition” as used herein, refers to any statistically significant decrease in biological activity, including full blocking of the activity. For example, “inhibition” can refer to a statistically significant decrease of about 10%, 20%, 30%, 40%, 50%, 60%, 70%, 80%, 90%, or about 100% in biological activity.

Inhibition of phosphorylation, as used herein, refers to the ability of an antibody to statistically significantly decrease the phosphorylation of a substrate protein relative to the signaling in the absence of the antibody (control). As is known in the art, intracellular signaling pathways include, for example, phosphoinositide 3′-kinase/Akt (PI3K/Akt/PTEN or “AKT”) and/or mitogen-activated protein kinase (MAPK/ERK or “ERK”) pathways. As is also known in the art, EGFR mediated signaling can be measured by assaying for the level phosphorylation of the substrate (e.g., phosphorylation or no phosphorylation of AKT and/or ERK). Accordingly, in one embodiment, anti-EGFR antibody combinations and compositions provide statistically significant inhibition of the level of phosphorylation of either or both of AKT and ERK by at least 10%, or at least 20%, or at least 30%, or at least 40%, or at least 50%, or at least 60%, or at least 70%, or at least 80%, or at least 90%, or about 100% relative to the level of phosphorylation of AKT and/or ERK in the absence of such antibody (control). Such EGFR mediated signaling can be measured using art recognized techniques which measure a protein in a cellular cascade involving EGFR, e.g., ELISA, western, or multiplex methods, such as Luminex®. The phrase “inhibition of EGFR downstream signaling” refers to the ability of an antibody or antibody mixture to reduce the level of signaling from the EGF receptor, as measured by, e.g., a reduction in the amount of phosphorylation of AKT and/or ERK.

The phrase “inhibition of cell growth,” as used herein, refers to the ability of an antibody or antibody mixture to statistically significantly decrease the growth of a cell or cells expressing EGFR relative to the growth of the cell or cells in the absence of the antibody (control) either in vivo or in vitro. In one embodiment, the growth of a cell expressing EGFR (e.g., a cancer cell) may be decreased by at least 10%, or at least 20%, or at least 30%, or at least 40%, or at least 50%, or at least 60%, or at least 70%, or at least 80%, or at least 90%, or about 100% when the cells are contacted with an antibody composition of combination disclosed herein, relative to the growth measured in the absence of the antibody composition of combination (control) or when the cells are contacted with a single species of monoclonal antibody. Cellular growth can be assayed using art recognized techniques which measure the rate of cell division, the fraction of cells within a cell population undergoing cell division, and/or the rate of cell loss from a cell population due to terminal differentiation or cell death (e.g., using a CellTiter-Glo® or similar assay).

The terms “treat,” “treating,” and “treatment,” as used herein, refer to therapeutic or preventative measures described herein. The methods of “treatment” employ administration to a subject, an antibody or antibody pair or trio disclosed herein, for example, a subject having a disorder associated with EGFR dependent signaling or predisposed to having such a disease or disorder, in order to prevent, cure, delay, reduce the severity of, or ameliorate one or more symptoms of the disease or disorder or recurring disease or disorder, or in order to prolong the survival of a subject beyond that expected in the absence of such treatment.

The term “patient” includes human and other mammalian subjects that receive either prophylactic or therapeutic treatment.

The term “sample” refers to tissue, body fluid, or a cell (or a fraction of any of the foregoing) taken from a patient. Normally, the tissue or cell will be removed from the patient, but in vivo diagnosis is also contemplated. In the case of a solid tumor, a tissue sample can be taken from a surgically removed tumor and prepared for testing by conventional techniques. In the case of lymphomas and leukemias, lymphocytes, leukemic cells, or lymph tissues can be obtained (e.g., leukemic cells from blood) and appropriately prepared. Other samples, including urine, tears, serum, plasma, cerebrospinal fluid, feces, sputum, cell extracts, malignant pleural effusion, etc. can also be useful for particular cancers.

Any biopsy technique used by those skilled in the art can be used for isolating a sample from a subject. The biopsy can be an open biopsy, in which general anesthesia is used or a closed biopsy, in which a smaller cut is made than in an open biopsy. The biopsy can be a core or incisional biopsy, in which part of the tissue is removed; an excisional biopsy, in which attempts to remove an entire lesion are made; or a needle aspiration (percutaneous) biopsy (fine needle aspiration biopsy), in which a sample of tissue or fluid is removed with a needle. The needle can be a thin, hollow needle, and it can be inserted into a mass to extract cells from the mass. In one embodiment, the sample is a tissue sample, e.g., a tissue sample obtained by surgical resection or biopsy of any kind (e.g., core needle biopsy, fine needle aspirate (FNA) biopsy, etc.). In another embodiment, the sample is a blood sample (e.g., plasma, serum, or whole blood sample) or is a urine sample.

The biopsy can be processed. For example, the biopsy can be preserved by formalin-fixing and paraffin-embedding (FFPE) the tissue. The biopsy can be processed into smaller pieces. The biopsy can be treated to preserve RNA. The biopsy can be stored on wet ice (approximately 4° C.), at room temperature (approximately 25° C.), stored at approximately −20° C., or at approximately −80° C., e.g., stored on dry ice, or frozen in liquid nitrogen or a dry ice/alcohol slurry. The tissue can be frozen within 0.5, 1, 5, 10, 15, 30, 60, 120, or 240 minutes of surgical resection. Fixative agents that can be used on the biopsy tissue include, for example, methanol-acetone, Carnoy's fixative (60% ethanol, 30% chloroform, 10% glacial acetic acid), Bouin's fixative, ethanol, acetone, formalin, methacarn (substitute 60% methanol for the ethanol in Carnoy), UMFIX (universal molecular fixative), Omnifix, and FINEfix.

As used herein, “antineoplastic agent” or “anti-cancer agent” refers to agents that have the functional property of inhibiting a development or progression of a neoplasm in a human, particularly a malignant (cancerous) lesion, such as a carcinoma, sarcoma, lymphoma, or leukemia. Inhibition of metastasis is frequently a property of antineoplastic agents.

As used herein, “Ras-wild type” refers to a cancer that does not harbor somatic mutations in exon 2 (codons 12 and 13), exon 3 (codons 59 and 61), and exon 4 (codons 117 and 146) of either K-Ras or N-Ras and hereafter is referred to as “Ras.”

As used herein, “BRAF wild type” refers to a cancer that does not harbor somatic mutations in codon 600 (e.g., V600E).

I. Anti-EGFR Antibodies

Compositions of anti-EGFR antibodies (or VH/VL domains derived therefrom) suitable for use in the invention can be generated using methods well known in the art. Alternatively, compositions comprising art recognized anti-EGFR antibodies, such as Sym-004 (Symphogen), cetuximab, panitumumab and nimotuzumab, can be used. Other pharmaceutical anti-EGFR antibodies include zalutumumab, and matuzumab, which are in development. Further anti-EGFR oligoclonal antibody compositions which can be employed are described in PCT International Publication No. WO/2011/140254 and corresponding pending U.S. Pat. No. 9,044,460, and in pending U.S. Pat. Nos. 9,226,964 and 7,887,805 (the “Oligoclonal Applications”), as well as oligoclonal mixtures of such antibodies in combination with other anti-EGFR antibodies, are useful for treatment of cancers, e.g., malignant (neoplastic) tumors. Antibodies that compete with any of these art-recognized antibodies for binding to EGFR also can be used.

An exemplary composition of anti-EGFR antibodies suitable for use in the invention is “MM-151”. MM-151 is an oligoclonal therapeutic consisting of a mixture of three fully human monoclonal antibodies designed to bind and inhibit signaling of the Epidermal Growth Factor Receptor (EGFR). MM-151 is a mixture of three independent antibodies (P1X+P2X+P3X), which to three non-overlapping sites on EGFR to maximize inhibition of ligand-dependent and independent signaling (see, e.g., WO 2013/006547, the teachings of which are expressly incorporated herein by reference). The P1X, P2X and P3X monoclonal antibodies are affinity matured antibodies of parental antibodies referred to as ca, cd and ch, respectively, disclosed in WO 2011/140254, the teachings of which are expressly incorporated herein by reference. The CDR amino acid sequences of P1X, P2X and P3X are shown below in Table 1:

TABLE 1
CDR amino acid sequences
of P1X, P2X and P3X

| Anti- | | | |
|---|---|---|---|
| body | CDR1 | CDR2 | CDR3 |
| P1X V_{H} | SYAIS | IIPIFGTVNY | DPSVNL |
| | SEQ ID NO: 1 | SEQ ID NO: 2 | SEQ ID NO: 3 |
| P1X V_{L} | QSISSWWA | DASSL | QQYHAHP |
| | SEQ ID NO: 4 | SEQ ID NO: 5 | SEQ ID NO: 6 |
| P2X V_{H} | SYAIS | IIPIFGAANP | MGRGKV |
| | SEQ ID NO: 7 | SEQ ID NO: 8 | SEQ ID NO: 9 |
| P2X V_{L} | QSVLYSPNNKNYLA | WASTR | QQYYGSP |
| | SEQ ID NO: 10 | SEQ ID NO: 11 | SEQ ID NO: 12 |
| P3X V_{H} | SYGIN | ISAYNGNTYY | DLGGYGSGS |
| | SEQ ID NO: 13 | SEQ ID NO: 14 | SEQ ID NO: 15 |
| P3X V_{L} | QSVSSNLA | GASTR | QDYRTWPR |
| | SEQ ID NO: 16 | SEQ ID NO: 17 | SEQ ID NO: 18 |

The full-length V_{H} and V_{L} amino sequences for P1X are shown in SEQ ID NO: 19 and SEQ ID NO: 20, respectively. The full-length V_{H} and V_{L} amino sequences for P2X are shown in SEQ ID NO: 21 and SEQ ID NO:22, respectively. The full-length V_{H} and V_{L} amino sequences for P3X are shown in SEQ ID NO: 23 and SEQ ID NO: 24, respectively. Additionally, the V_{H} and V_{L} CDR segments as presented herein are arranged, e.g., in the amino to carboxy terminal order of CDR1, CDR2 and CDR3.

In one embodiment, the composition of anti-EGFR antibodies comprises: (1) a monoclonal antibody comprising heavy chain CDR1, CDR2, and CDR3 sequences of SEQ ID NOs: 1, 2, and 3, respectively, and light chain CDR1, CDR2, and CDR3 sequences of SEQ ID NOs: 4, 5, and 6, respectively; (2) a monoclonal antibody comprising heavy chain CDR1, CDR2, and CDR3 sequences of SEQ ID NOs: 7, 8, and 9, respectively, and light chain CDR1, CDR2, and CDR3 sequences of SEQ ID NOs: 10, 11, and 12, respectively; and (3) a monoclonal antibody comprising heavy chain CDR1, CDR2, and CDR3 sequences of SEQ ID NOs: 13, 14, and 15, respectively, and light chain CDR1, CDR2, and CDR3 sequences of SEQ ID NOs: 16, 17, and 18, respectively.

In another embodiment, the composition of anti-EGFR antibodies comprises: (1) a monoclonal antibody comprising a heavy chain variable region comprising SEQ ID NO: 19; (2) a monoclonal antibody comprising a heavy chain variable region comprising SEQ ID NO: 21 and (3) a monoclonal antibody comprising a heavy chain variable region comprising SEQ ID NO: 23.

In another embodiment, the composition of anti-EGFR antibodies comprises: (1) a monoclonal antibody comprising a light chain variable region comprising SEQ ID NO: 20; (2) a monoclonal antibody comprising a light chain variable region comprising SEQ ID NO: 22; and (3) a monoclonal antibody comprising a light chain variable region comprising SEQ ID NO: 24.

In another embodiment, the composition of anti-EGFR antibodies comprises: (1) a monoclonal antibody comprising a heavy chain variable region comprising SEQ ID NO: 19 and a light chain variable region comprising SEQ ID NO: 20; (2) a monoclonal antibody comprising a heavy chain variable region comprising SEQ ID NO: 21 and a light chain variable region comprising SEQ ID NO: 22; and (3) a monoclonal antibody comprising a heavy chain variable region comprising SEQ ID NO: 23 and a light chain variable region comprising SEQ ID NO: 24.

In another embodiment, the anti-EGFR antibodies (1), (2), and (3) are in the composition at a molar ratio of 2:2:1 to each other.

The anti-EGFR antibodies can be administered alone or with another therapeutic agent that acts in conjunction with or synergistically with the oligoclonal antibody to treat the disease associated with EGFR-mediated signaling.

In another embodiment, the treatment methods described herein comprise administering the anti-EGFR antibodies in combination with one or more other antineoplastic agents (e.g., other chemotherapeutics or other small molecule drugs). In one embodiment, no more than three other antineoplastic agents are administered within a treatment cycle. In another embodiment, no more than two other antineoplastic agents are administered within a treatment cycle. In another embodiment, no more than one other antineoplastic agent is administered within a treatment cycle. In another embodiment, no other antineoplastic agent is administered within a treatment cycle.

As used herein, adjunctive or combined administration (coadministration) includes simultaneous administration the anti-EGFR antibodies and one or more antineoplastic agents in the same or different dosage form, or separate administration of the anti-EGFR antibodies and one or more antineoplastic agents (e.g., sequential administration). Such concurrent or sequential administration preferably results in both the anti-EGFR antibodies and the one or more agents being simultaneously present in treated patients.

II. Methods of Treatment

Provided herein are methods for treating cancer in a human patient. In one embodiment, the cancer is a tumor of the skin, central nervous system, head, neck, esophagus, stomach, colon, rectum, anus, liver, pancreas, bile duct, gallbladder, lung, breast, ovary, uterus, cervix, vagina, testis, germ cells, prostate, kidney, ureter, urinary bladder, adrenal, pituitary, thyroid, bone, muscle or connective tissue. In another embodiment, the cancer is selected from the group consisting of colorectal cancer, metastatic colorectal cancer, non-small cell lung cancer, squamous cell head and neck cancer, or recurrent or metastatic head and neck cancer. In another embodiment, the patient has been diagnosed with K-Ras wild-type metastatic colorectal cancer. In another embodiment, the cancer is an EGFR extracellular domain (ECD) mutant cancer, such as a K-Ras, N-Ras, and/or BRAF wild-type metastatic colorectal cancer. In another embodiment, the mutant cancer is K-Ras wild-type, EGFR-expressing metastatic colorectal cancer as determined by an FDA-approved test. In another embodiment, the mutant cancer is a head and neck cancer (e.g., locally or regionally advanced squamous cell carcinoma of the head and neck). In another embodiment, the mutant cancer is Ras-wild type wild-type KRAS (exon 2) metastatic colorectal cancer (mCRC) as determined by an FDA-approved test.

In another embodiment, the tumor is determined to comprise cells that harbor at least one mutation in an EGFR extracellular domain based on an FDA-approved test. In another embodiment, the cancer is a tumor comprising cells harboring a mutation in Domain III or Domain IV of the EGFR extracellular domain. In another embodiment, the cancer is a tumor containing cells harboring a mutation in exon 12 of the EGFR gene. In another embodiment, the at least one mutation in the extracellular domain of EGFR, which is a protein sequence change, or a DNA or RNA coding region that results in a protein sequence change, selected from the group consisting of EGFR R451C, S464L, K467T, G465R, G465E, I491M, and S492R. In one embodiment, the mutation in the extracellular domain of EGFR is arginine to cysteine at position 451 of SEQ ID NO: 27 (i.e., the sequence corresponding to the extracellular domain of EGFR). In another embodiment, the mutation in the extracellular domain of EGFR is serine to leucine at position 464 of SEQ ID NO: 27. In another embodiment, the mutation in the extracellular domain of EGFR is lysine to threonine at position 467 of SEQ ID NO: 27. In another embodiment, the mutation in the extracellular domain of EGFR is glycine to arginine at position 465 of SEQ ID NO: 27. In another embodiment, the mutation in the extracellular domain of EGFR is glycine to glutamate at position 465 of SEQ ID NO: 27. In another embodiment, the mutation in the extracellular domain of EGFR is isoleucine to methionine at position 491 of SEQ ID NO: 27. In another embodiment, the mutation in the extracellular domain of EGFR is serine to arginine at position 492 of SEQ ID NO: 27.

In one aspect, methods of treating a patient having a tumor comprising cells that harbor at least one mutation in an EGFR extracellular domain are provided, the method comprising administering to the patient an effective amount of a monoclonal antibody mixture that binds to an EGFR extracellular domain comprising the at least one mutation, wherein the method comprises administering to the patient in a single composition an oligoclonal mixture of anti-EGFR antibodies that bind to the EGFR extracellular domain, as described herein.

In another aspect, the methods include treating a patient who has progressed on or become refractory to one or more of a monoclonal antibody that binds to the EGFR extracellular domain (e.g., cetuximab or panitumumab). In another embodiment the patient has progressed on or become refractory to an antineoplastic therapy after prior treatment with an antineoplastic agent. In another embodiment, the patient has become resistant to an antineoplastic therapy after prior treatment with an antineoplastic agent. In another embodiment, the patient is treated following disease progression or recurrence after prior treatment with an antineoplastic agent. In another embodiment, the patient is treated after failure of treatment with an antineoplastic agent. In another embodiment, the cancer is identified as a cancer that has acquired resistance to an antineoplastic agent.

In another aspect, the methods include treating a patient who has had disease progression on or following a fluoropyrimidine-containing regimen, an oxaliplatin-containing regimen, or an irinotecan-containing regimen.

In another aspect, the methods include treating an epidermal growth factor receptor (EGFR) extracellular domain (ECD) mutant cancer in a human patient by administering to the patient an oligoclonal anti-epidermal growth factor receptor (anti-EGFR) antibody combination (e.g., MM-151).

In another aspect, the methods include treating an epidermal growth factor receptor (EGFR) extracellular domain (ECD) mutant cancer in a human patient by (a) detecting a mutation in exon 12 of the ECD of EGFR in the patient (e.g., in the blood of the patient); and then (b) administering to the patient a therapeutically effective amount of an oligoclonal anti-epidermal growth factor receptor (anti-EGFR) antibody combination (e.g., MM-151).

In another aspect, the methods include treating an epidermal growth factor receptor (EGFR) extracellular domain (ECD) mutant cancer in a human patient, wherein the cancer is selected from the group consisting of: head and neck cancer and colorectal cancer by: (a) detecting a mutation in Domain III or Domain IV of the EGFR extracellular domain (e.g., in the blood of the patient); and then (b) administering to the patient a therapeutically effective amount of a MM-151 oligoclonal anti-epidermal growth factor receptor (anti-EGFR) antibody combination (e.g., MM-151). In another aspect, the methods include treating a human patient having an epidermal growth factor receptor (EGFR) extracellular domain (ECD) mutant cancer having a detected mutation in Domain III or Domain IV of the ECD of EGFR, the method comprising administering to the patient a therapeutically effective amount of a MM-151 oligoclonal anti-epidermal growth factor receptor (anti-EGFR) antibody combination, wherein the oligoclonal antibody combination consists of a P1X monoclonal antibody, a P2X monoclonal antibody, and a P3X monoclonal antibody in a 2:2:1 molar ratio.

In a further aspect, the methods include treating a human patient having an epidermal growth factor receptor (EGFR) extracellular domain (ECD) mutant cancer having at least one detected mutation in the ECD of EGFR selected from the group consisting of EGFR R451C, S464L, K467T, G465R, G465E, I491M, and S492R, the method comprising administering to the patient an oligoclonal anti-epidermal growth factor receptor (anti-EGFR) antibody combination comprising three monoclonal antibodies, wherein the first monoclonal antibody is P1X, the second monoclonal antibody is P2X, the third monoclonal antibody is P3X, and the oligoclonal antibody combination comprises P1X, P2X and P3X in a 2:2:1 molar ratio.

In a further aspect, the methods including preventing emergence or inhibiting growth of an epidermal growth factor receptor (EGFR) extracellular domain (ECD) mutant cancer in a human patient by administering to the patient an oligoclonal anti-epidermal growth factor receptor (anti-EGFR) antibody combination (e.g., MM-151). In one embodiment, the oligoclonal anti-epidermal growth factor receptor (anti-EGFR) antibody combination is administered to a patient who has not received prior anti-EGFR therapy (e.g., treatment with cetuximab, panitumumab, or Sym004). In one embodiment, the oligoclonal anti-epidermal growth factor receptor (anti-EGFR) antibody combination is administered to a patient who has received prior anti-EGFR therapy (e.g., treatment with cetuximab, panitumumab, or Sym004). In one embodiment, the oligoclonal anti-epidermal growth factor receptor (anti-EGFR) antibody combination is administered immediately after detection of an EGFR ECD mutation (e.g., within hours or days after detection).

The presence of EGFR mutations can be detected in any suitable biological sample (e.g., tissue, blood, urine, etc.) using known techniques. For example, in one embodiment, an EGFR mutation is detected using polymerase chain reaction (PCR)-based methods, including, but not limited to real-time PCR with allele-specific primers, real-time PCR with allele-specific TaqMan probes, quantitative sequencing (PCR-based sequencing), droplet digital PCR (ddPCR), BEAMing Digital PCR, and improved and complete enrichment CO-amplification at lower denaturation temperature (ICE COLD)-PCR. In another embodiment, an EGFR mutation is detected using Next Generation Sequencing (NGS) as described, for example, by Jorge S Reis-Filho (Breast Cancer Research, 2009, 11(Suppl 3):S12). NGS (also known as “high-throughput sequencing” or “massively parallel sequencing”), is a term used to describe a number of different modern sequencing technologies including, but not limited to IIlumina (Solexa) sequencing, Roche 454 sequencing, Ion torrent: Proton/PGM sequencing, and SOLiD sequencing. Other platforms for analyzing EGFR mutations include, for example, Sanger Sequencing, single nucleotide polymorphism (SNP) arrays, high pressure liquid chromatography (HPLC), mass-spectrometry, FISH, etc. Exemplary assays for measuring EGFR ECD domain mutations include, but are not limited to FoundationOne (Foundation Medicine, NGS), OncoBEAM (Sysmex Inostics, PCR), Guardant360, (Guardant Health, NGS), Trovagene Precision Cancer Monitoring (PCM) (Trovagene, PCR), MX-ICP EGFR exon 12 (S492R) (Transgenomic, PCR), and OmniSeq Comprehensive Panel (Omniseq LLC, NGS).

Determining the presence or absence of an EGFR mutation (e.g., mutation in EGFR exon 12) can be performed in a variety of ways. Such tests are commonly performed using DNA or RNA collected from biological samples. In one embodiment, assays for detecting such mutations can be performed according to any suitable protocol and/or standard manufacturer instructions. In one embodiment, NGS-based methods for detecting mutations in EGFR exon 12 provides full coverage of exon 12. In another embodiment, PCR-based methods provide codon-specific assays for all codons in exon 12. In another embodiment, other methods are use to assess exon 12. In a particular embodiment where an assay does not cover all of exon 12, the assay preferably at least assesses all known exon 12 mutations. However, if a limited assay assessing only a subset of mutations is capable of identifying a mutation, it is sufficient to determine EGFR-ECD mutation positivity. With respect to assay sensitivity, EGFR-ECD mutation status is determined by the presence of the mutation above the limit of detection of the assay. It is not necessary to use a cut point (threshold) higher than the limit of detection (i.e., a minimum level of EGFR mutation). Published reports on EGFR-ECD allelic frequency (a measure of % mutant DNA vs wild type DNA) show levels as low as 0.1%. In one embodiment, a suitable assay should have a limit of detection<1% (preferably, lower) to prevent potential false negatives. In a preferred embodiment, the assay is capable of being certified by the Clinical Laboratory Improvement Amendments (CLIA) (i.e., a CLIA certified assay).

Treatment outcomes can be evaluated using standard measures for tumor response. Target lesion (tumor) responses to therapy are classified as:

Complete Response (CR): Disappearance of all target lesions. Any pathological lymph nodes (whether target or non-target) must have reduction in short axis to <10 mm;

Partial Response (PR): At least a 30% decrease in the sum of the diameters of target lesions, taking as reference the baseline sum diameters;

Progressive Disease (PD: At least a 20% increase in the sum of the diameters of target lesions, taking as reference the smallest sum on study (this includes the baseline sum if that is the smallest on study). In addition to the relative increase of 20%, the sum must also demonstrate an absolute increase of at least 5 mm. (Note: the appearance of one or more new lesions is also considered progression); and

Stable Disease (SD): Neither sufficient shrinkage to qualify for PR nor sufficient increase to qualify for PD, taking as reference the smallest sum diameters while on study. (Note: a change of 20% or less that does not increase the sum of the diameters by 5 mm or more is coded as stable disease). To be assigned a status of stable disease, measurements must have met the stable disease criteria at least once after study entry at a minimum interval of 6 weeks.

Non-target lesion responses to therapy are classified as:

Complete Response (CR): Disappearance of all non-target lesions and normalization of tumor marker levels. All lymph nodes must be non-pathological in size (<10 mm short axis). If tumor markers are initially above the upper normal limit, they must normalize for a patient to be considered in complete clinical response;

Non-CR/Non-PD: Persistence of one or more non-target lesion(s) and/or maintenance of tumor marker level above the normal limits; and

Progressive Disease (PD): Either or both of appearance of one or more new lesions and unequivocal progression of existing non-target lesions. In this context, unequivocal progression must be representative of overall disease status change, not a single lesion increase.

Patients treated according to the methods disclosed herein preferably experience improvement in at least one sign of cancer. For example, the treatment may produce at least one therapeutic effect selected from the group consisting of reduction in size of a tumor, reduction in metastasis, complete remission, partial remission, stable disease, increase in overall response rate, or a pathologic complete response. Response may also be measured by a reduction in the quantity and/or size of measurable tumor lesions. Measurable lesions are defined as those that can be accurately measured in at least one dimension (longest diameter is to be recorded) as >10 mm by CT scan (CT scan slice thickness no greater than 5 mm), 10 mm caliper measurement by clinical exam or >20 mm by chest X-ray. The size of non-target lesions, e.g., pathological lymph nodes can also be measured for improvement. Lesions can be measured using, e.g., x-ray, CT, or MRI images. Microscopy, cytology or histology can be also used to evaluate responsiveness to a therapy. An effusion that appears or worsens during treatment when a measurable tumor has otherwise met criteria for response or stable disease can be considered to indicate tumor progression, but only if there is cytological confirmation of the neoplastic origin of the effusion.

In another embodiment, the patient so treated experiences tumor shrinkage and/or decrease in growth rate, i.e., suppression of tumor growth. In another embodiment, tumor cell proliferation is reduced or inhibited. Alternately, one or more of the following can indicate a beneficial response to treatment: the number of cancer cells can be reduced; tumor size can be reduced; cancer cell infiltration into peripheral organs can be inhibited, retarded, slowed, or stopped; tumor metastasis can be slowed or inhibited; tumor growth can be inhibited; recurrence of tumor can be prevented or delayed; one or more of the symptoms associated with cancer can be relieved to some extent. Other indications of a favorable response include reduction in the quantity and/or size of measurable tumor lesions or of non-target lesions.

III. Methods of Selection

Also provided herein are methods of selecting treatment for a patient having a tumor that expresses EGFR, wherein the patient is refractory to antibody based anti-EGFR therapy.

In one embodiment, the method comprises a) determining whether cells from the tumor harbor at least one mutation in the extracellular domain of EGFR; and b) if so, selecting treatment comprising the anti-EGFR antibody compositions described herein. In a further embodiment, the method comprises obtaining a biopsy sample of the tumor, and determining whether the tumor has at least one mutation in the DNA or RNA coding for the extracellular domain of EGFR using methods comprising polymerase chain reaction or next-generation sequencing. In a further embodiment, the method comprises obtaining a biopsy sample of the tumor, and determining whether the tumor has at least one mutation in the extracellular domain of EGFR (e.g., via immunohistochemistry or mass spectrometry). In a further embodiment, the method comprises obtaining a blood sample from the patient, and determining whether the blood sample contains circulating DNA (e.g., cell-free DNA isolated from the blood sample or DNA isolated from circulating tumor cells in the blood sample) with one or more mutations in sequences encoding the extracellular domain of EGFR. In a further embodiment, the biopsied tumor cells, circulating tumor cells, or cell-free DNA harbor at least one mutation in DNA or RNA coding for the extracellular domain of EGFR, and the at least one mutation is in exon 12 of the EGFR gene. In a further embodiment, the at least one mutation in the extracellular domain of EGFR is a protein sequence change, or a DNA or RNA coding region that results in a protein sequence change, chosen from the group consisting essentially of EGFR R451C, S464L, K467T, G465R, I491M, and S492R. In a further embodiment, the tumor is determined to comprise cells that harbor at least one mutation in an EGFR extracellular domain as detected by an FDA-approved test.

IV. Methods of Prediction

Also provided herein are methods of determining whether or not an oligoclonal antibody mixture may be used to treat patients having a cancer that is refractory to treatment with one or more monoclonal anti-EGFR antibody preparations comprising only a single species of anti-EGFR antibody. Use of oligoclonal anti-EGFR antibodies for the treatment of a cancer is described, wherein the cancer expresses EGFR and the EGFR is determined to have at least one mutation in the extracellular domain.

In one embodiment, methods of predicting which tumors (e.g., malignant tumors) will be responsive to treatment with oligoclonal anti-EGFR antibodies, but not responsive to treatment with single monoclonal anti-EGFR antibodies (e.g., cetuximab, panitumumab), the methods comprising determining whether a patient's tumor cells have acquired mutations in the EGFR gene region coding for the extracellular domain.

Other embodiments are described in the following non-limiting Examples. The following examples are merely illustrative and should not be construed as limiting the scope of this disclosure in any way as many variations and equivalents will become apparent to those skilled in the art upon reading the present disclosure.

The contents of all references, Genbank entries, patents and published patent applications cited throughout this application are expressly incorporated herein by reference.

### EXAMPLES

### Materials and Methods

Throughout the examples, the following materials and methods are used unless otherwise stated.

In general, unless otherwise indicated, conventional techniques of chemistry, molecular biology, recombinant DNA technology, immunology (especially, e.g., antibody technology), and standard techniques in polypeptide preparation are used. See, e.g., Sambrook, Fritsch and Maniatis, Molecular Cloning Cold Spring Harbor Laboratory Press (1989); Antibody Engineering Protocols (Methods in Molecular Biology), 510, Paul, S., Humana Pr (1996); Antibody Engineering: A Practical Approach (Practical Approach Series, 169), McCafferty, Ed., Irl Pr (1996); Antibodies: A Laboratory Manual, Harlow et al., C.S.H.L. Press, Pub. (1999); and Current Protocols in Molecular Biology, eds. Ausubel et al., John Wiley & Sons (1992).

### Cell Culture and Generation of Resistant Cells

OXCO-2 cells were cultured in Iscove medium (Invitrogen) supplemented with 5% FBS; LIM1215 cells were cultured in RPMI1640 medium (Invitrogen) supplemented with 5% FBS and insulin (1 mg/mL); HCA-46 cells were cultured in DMEM (Invitrogen) supplemented with 5% FBS and CCK81 cells were cultured in MEM (Invitrogen) supplemented with 5% FBS. All media also contained 2 mmol/L L-glutamine and antibiotics (100 U/mL penicillin and 100 mg/mL streptomycin), and cells were grown in a 37° C. and 5% CO2 air incubator. The OXCO-2 cell lines were a kind gift from Dr V. Cerundolo in March 2010 (Weatherall Institute of Molecular Medicine, University of Oxford, Oxford, United Kingdom). The LIM1215 parental cell line has been described previously (R. H. Whitehead, F. A. Macrae, D. J. St John, J. Ma, A colon cancer cell line (LIM1215) derived from a patient with inherited nonpolyposis colorectal cancer. J. Natl. Cancer Inst. 74, 759-765 (1985)) and was obtained from Prof. Robert Whitehead (Vanderbilt University, Nashville, Tenn.) with permission from the Ludwig Institute for Cancer Research (Zurich, Switzerland). HCA-46 cell lines were obtained from European Collection of Animal Cell Cultures (distributed by Sigma-Aldrich Srl). CCK81 cell line was obtained from Health Science Research Resources Bank. The HEK293 cell line was purchased from ATCC (LGC Standards S.r.l) and cultured in DMEM (Invitrogen) supplemented with 10% FBS.

The identity of each cell line was tested and authenticated by Cell ID System and by Gene Print 10 System (Promega), through short tandem repeats (STR) at 10 different loci (D5S818, D13S317, D7S820, D16S539, D21S11, vWA, TH01, TPDX, CSF1PO, and amelogenin). Amplicons from multiplex PCRs were separated by capillary electrophoresis (3730 DNA Analyzer, Applied Biosystems) and analyzed using GeneMapperlD software from Life Technologies. Resulting cell line STR profiles were cross-compared and matched with the available STR from ATCC, ECACC, and CellBank Australia repositories online databases. All cell lines were tested and resulted negative for mycoplasma contamination with Venor GeM Classic Kit (Minerva Biolabs).

LIM1215 R1 were generated by continuous exposure to the drug at a concentration of 1.4 μM. LIM1215 R2 were obtained by increasing the cetuximab dosage stepwise from 350 nM to 1.4 μM for LIM1215. LIM1215 R3-5 and OXCO-2, and HCA-46 cetuximab-resistant derivatives were generated after 3-9 months of continuous exposure to the drug at a concentration of 0.3 μM for NCIH508 and 1.4 μM for LIM1215, OXCO-2 and HCA-46. CCK81 cetuximab-resistant derivatives were obtained by increasing the cetuximab dosage stepwise from 680 nM to 1.4 μM during the course of 6 months.

### Mutational Analysis in Cell Lines

Genomic DNA samples were extracted by Wizard SV Genomic DNA Purification System (Promega). For Sanger sequencing, all samples were subjected to automated sequencing by ABI PRISM 3730 (Applied Biosystems). Primer sequences are listed elsewhere (6, 9). The following genes and exons were analyzed: KRAS (exons 2, 3, and 4), NRAS (exons 2 and 3), PIK3CA (exons 9 and 20), BRAF (exon 15), EGFR (exon 12). All mutations were confirmed twice, starting from independent PCR reactions.

### Generation of KI Cells (LIM1215 KI EGFR S492R)

LIM1215 cells were grown in a 10 cm dish until 60-80% confluence. Then the media was aspirated and 2 μl of rAAV (pAAV0223 EGFR S492R, Horizon Discovery, Cambridge, UK) and 5 ml of the fresh appropriate growth media were added to the cells. The virus was allowed to infect cells at 37° C. for 4 hours. Afterwards 5 ml of growth media were added to the cells and then completely renovated with fresh media after 14-16 hours. Infected cells were allowed to grow for 48 hours and then harvested by trypsinization and distributed in 96-well plates with geneticin (G418, Life Technologies Corporation, Grand Island, N.Y.) containing media. The plates were incubated at 37° C. for 2 weeks prior to harvest and screening for recombinants. Locus specific homologous recombination events were screened by “Neo screening” PCR using a forward primer located outside of the left homology arm, FW: 5′ AAGATGGGGGAAAGAAGAGC 3′ (SEQ ID NO: 28), and a reverse primer located within the Neo gene, RV: 5′ GCATGCTCCAGACTGCCTTG 3′ (SEQ ID NO: 29). Genomic DNA from single clones was obtained by lysing cells with Lyse-N-Go™ PCR Reagent (Pierce, Rockford, Ill.) following manufacturer instructions. PCR screening was carried out in 10 μl reaction volumes using the following conditions: 1 cycle of 94° C. for 3 min; 3 cycles of 94° C. for 15 s, 64° C. for 30 s, 70° C. for 1 min and 30 sec; 3 cycles of 94° for 15 s, 61° C. for 30 s, 70° C. for 1 min and 30 sec; 3 cycles of 94° C. for 15 s, 58° C. for 30 s, 70° C. for 1 min and 30 sec; 35 cycles of 94° C. for 15 s, 55° C. for 30 s, 70° for 1 min and 30 sec; 1 cycle of 70° C. for 5 min. In positive clones, the introduction and expression of the S492R mutation was checked by sequencing at a gDNA and cDNA level.

To remove the LoxedP Neo cassette, LIM1215 EGFR S492R knock-in cells were incubated with 1 μl of the Cre Recombinase Adenovirus in 5 mL of the appropriate growth media. The virus was allowed to infect cells at 37° C. for 4 hours. Then 5 ml of growth media were added to the cells and then completely renovated with fresh media after 14-16 hours. Cells were then seeded in 96-well plates at limiting dilution for isolating single clones. When single clones reached 50-60% of confluence, they was harvested and the genomic DNA was extracted from each well using the Lyse-N-Go PCR™ Reagent, as described in the previous paragraph, and a double PCR screening was performed to assess the removal of the Neo cassette. The first PCR reaction, supposed to be negative, was similar to the “Neo screening” as described above. It was carried out using primers annealing to the genomic DNA, FW: 5′ AAGATGGGGGAAAGAAGAGC 3′, (SEQ ID NO: 30) and the neomycin gene, RV: 5′ GCATGCTCCAGACTGCCTTG 3′ (SEQ ID NO: 31). This PCR was negative for CRE out clones. The second PCR reaction was expected to check for the presence of the mutation. The PCR reactions were performed in 10 μl and in 20 μl reaction volumes respectively, using the same conditions, cycling times and temperatures noted above for neo screening. To prove the removal of the Neo cassette also at a functional level, the selected clones were seeded in both G418-containing (0.8 mg/mL) and G418-free media. As expected, CRE out clones were able to survive only in G418-free media. These clones were then subjected to expansion and final mutational analysis.

### Generation of the LIM1215 (#2) EGFR S492R Cell Line

LIM1215 cells were acquired from an additional cell bank (Sigma-Aldrich) and these cells are herein termed “LIM1215 (#2).” The cells were authenticated by short tandem repeat profiling by the manufacturer and passaged in our laboratory for less than 3 months according to manufacturer's instructions. LIM1215 (#2) cells were cultured in RPMI1640 medium (Life Technologies) supplemented with 10% FBS and 1% penicillin/streptomycin (Life Technologies) in a humidified incubator at 37° C. in a 5% CO₂ atmosphere. A pD2539 stable expression construct expressing EGFR harboring the S492R mutation from an EF1α promoter was acquired from DNA 2.0. This construct was generated by DNA 2.0 in a multi-step process. First, full-length EGFR was generated via DNA synthesis with the following silent mutations—removal of two internal BsaI restriction enzyme sites, generation of a short cloning cassette between bases 1253 and 1572 (this includes all of EGFR exon 12) by adding two BsaI directional endonuclease sites, and insertion of unique “mod1a” and “mod2a” qPCR primer binding sites to monitor expression. Second, this EGFR sequence was then inserted into a PM269 vector to generate the “PM269-EGFR” construct. Third, a cloning cassette containing the S492R mutation (1474A>C) was then synthesized and inserted into the PM269-EGFR construct using the two inserted BsaI directional endonuclease sites. Fourth, the Electra sub-cloning system (DNA 2.0) was then used to transfer the modified EGFR sequence (SEQ ID NO: 33) into a pD2539 stable expression vector, herein termed “pD2539-EGFR-S492R.”

LIM1215 (#2) cells were transfected with pD2539-EGFR-S492R using FuGENE® 6 Transfection Reagent (Roche cat#11-814-443-001). Transfected cells were grown in RPMI1640 medium (Life Technologies) supplemented with 10% FBS and 1 μg/mL puromycin (Life Technologies) for two weeks to generate a stable expression pool. RNA was collected and extracted using the RNeasy plus mini kit (Qiagen) and reverse transcription performed using the high-capacity cDNA reverse transcription kit (Life Technologies). SYBR green real time PCR was performed on a ViiA7 instrument (Life Technologies) to confirm expression of EGFR S492R mRNA using modla primers (5′GGGTTCGCATCAGATCTCGTTA3′ (SEQ ID NO: 36); 5′TCAAGCACCTATGAACTCGGAC3′ (SEQ ID NO: 37)). Expression was normalized to β-actin housekeeping control using primers obtained from Qiagen (Hs_ACTB_1_SG).

The LIM1215 (#2) cell line harboring stable expression of the pD2539-EGFR-S492R plasmid was then plated in 10 cm culture plates at a starting density of 50 cells per plate and grown under selection pressure with 1 μg/mL puromycin. Media was changed every five days and 24 single clones were then picked with cloning discs and allowed to expand under selection pressure. Expression of S492R mRNA was then assessed in the 24 clones and the clone with the highest expression selected for further experiments. Expression of total EGFR protein was measured by immunoblot and found to be approximately 5.7 fold higher than the parental LIM1215 (#2) cell line. This clone is herein termed “LIM1215 (#2) EGFR S492R.”

### Generation of the LIM1215 (#2) EGFR S492R KI Cell Line

Single guide RNA (sgRNA) sequences were designed using the CRISPR Design Tool (Massachusetts Institute of Technology; http://crispr.mit.edu) to target the EGFR S492 region (“S492”; 5′AATTTTGGTTTTCTGACCGG3′ (SEQ ID NO: 38)) and, as a control, a non-specific sequence (“rg_0111”; 5′ACGGAGGCTAAGCGTCGCAA3′ (SEQ ID NO: 39)). The two sgRNA sequences were separately cloned into a mammalian Cas9 genome editing vector (pD1321-AD, CAG-Cas9-2A-GFP) obtained from DNA 2.0 and are herein termed “pD1321-AD-S492R” and “pD1321-AD-rg_0111,” respectively. A single-stranded donor oligonucleotide containing the EGFR S492R mutation was synthesized by Integrated DNA Technologies (SEQ ID NO: 34).

For each plasmid (pD1321-AD-S492R and pD1321-AD-rg_0111), LIM1215 (#2) cells were transiently transfected with both plasmid and the EGFR S492R single-stranded donor oligonucleotide (SEQ ID NO: 34). Transfected cells were sorted by flow cytometry based on expression of the GFP protein encoded on the plasmid. Genomic DNA (gDNA) was extracted using the QIAamp® DNA Mini Kit from Qiagen (catalog #51306) according to manufacturer's instructions. gDNA was eluted in 50 μL of elution buffer (10 mM Tris-C1, 0.5 mM EDTA, pH 9.0) and gDNA concentration measured on a NanoDrop® 2000C UV-Vis spectrophotometer (ThermoScientific) according to manufacturer's instructions. Presence of the S492R mutation was confirmed by Sanger sequencing.

Single cells from the pooled culture were isolated into individual wells of a 96-well plate and gDNA was extracted for genotyping. Single clones identified to contain the EGFR S492R mutation were banked and selected for further experiments. A flow cytometry antibody binding assay was performed to identify a clone that reduces cetuximab binding while maintaining P2X binding (both relative to binding to the LIM1215 (#2) cell line expressing wild type EGFR). This clone is herein termed “LIM1215 (#2) EGFR S492R KI.”

A flow cytometry experiment was performed with the pooled culture generated with the pD1321-AD-rg_0111 plasmid (non-targeting sequence that maintains wild type EGFR genotype) to confirm that binding of cetuximab and P2X antibodies is maintained relative to the LIM1215 (#2) cell line expressing wild type EGFR. This pooled culture is herein termed “LIM1215 (#2) EGFR S492 wild type.”

### Generation of the LIM1215 (#2) EGFR G465R KI Cell Line

Single guide RNA (sgRNA) sequences were designed using the CRISPR Design Tool (Massachusetts Institute of Technology; http://crispr.mit.edu) to target the EGFR G465 region (“G465sg1”; 5′CTCCATCACTTATCTCCTTG3′ (SEQ ID NO: 40); “G465sg2”; 5′GCTATGCAAATACAATAAAC3′ (SEQ ID NO: 41)) and, as a control, a non-specific sequence (“dual-rg0111”; 5′ACGGAGGCTAAGCGTCGCAA3′ (SEQ ID NO: 42); “dual-rg-0135”; 5′CGCTTCCGCGGCCCGTTCAA3′ (SEQ ID NO: 43)). The two sgRNA sequences were cloned into a mammalian nikase Cas9-D10A genome editing vector (pD1431-AD, EF1a-Cas9N-2A-GFP) obtained from DNA 2.0 and are herein termed “pD1431-AD-nCas9G465R” and “pD1431-AD-nCas9 non_targeting,” respectively. A single-stranded donor oligonucleotide containing the EGFR G465R mutation was synthesized by Integrated DNA Technologies (SEQ ID NO: 35).

For each plasmid (pD1431-AD-nCas9G465R and pD1431-AD-nCas9 non_targeting), LIM1215 (#2) cells were transiently transfected with both plasmid and the EGFR G465R single-stranded donor oligonucleotide (SEQ ID NO: 35). Transfected cells were sorted by flow cytometry based on expression of the GFP protein encoded on the plasmid. Genomic DNA (gDNA) was extracted using the QIAamp® DNA Mini Kit from Qiagen (catalog #51306) according to manufacturer's instructions. gDNA was eluted in 50 μL of elution buffer (10 mM Tris-Cl, 0.5 mM EDTA, pH 9.0) and gDNA concentration measured on a NanoDrop® 2000C UV-Vis spectrophotometer (ThermoScientific) according to manufacturer's instructions. The presence of the G465R mutation was confirmed by Sanger sequencing.

Analysis of EGFR ECD Mutations in an In Vitro Pooled Culture System with a Mixture of Cells Expressing Wild Type EGFR and Cells Expressing EGFR ECD Mutations.

On the first day of the experiment, a pool of cells harboring expression of both wild type EGFR and an EGFR ectodomain mutation is seeded at a density of 1×10⁵ cell/well into 24-well plates. Two pools were generated during development of the LIM1215 (#2) EGFR S492R KI and the LIM1215 (#2) EGFR G465R KI cell lines that contains cells that harbor the EGFR S492R or EGFR G465R gene edit, respectively. Cells were cultured in RPMI1640 medium (Life Technologies) supplemented with 10% FBS and 1% penicillin/streptomycin (Life Technologies) in a humidified incubator at 37° C. in a 5% CO₂ atmosphere. As a negative control, a pool of cells expressing only wild type EGFR were plated and cultured in the same manner (this pool was generated during the development of the LIM1215 (#2) EGFR S492 wild type control cell line).

Also on the first day, 5×10⁵ cells were pelleted at 3000 RPM for 5 minutes in a benchtop centrifuge, supernatant was removed, and the cell pellet was stored at −80° C. for later genomic DNA extraction. The following day, cells were treated with 1 μM concentrations of antibody or media alone (control). On days 4, 10, and 15, half of the cells in each plate well were transferred to a new plate and half of the cells were used to form cell pellets, as above, for later genomic DNA extraction.

After all cell pellets were collected, samples were thawed, resuspended in 200 μL of phosphate-buffered saline (PBS), and genomic DNA extracted using the QIAamp DNA Mini Kit from Qiagen (catalog #51306) according to manufacturer's instructions. Genomic DNA was eluted in 50 μL of elution buffer (10 mM Tris-Cl, 0.5 mM EDTA, pH 9.0) and gDNA concentration measured on a NanoDrop® 2000C UV-Vis spectrophotometer (ThermoScientific) according to manufacturer's instructions.

The allelic frequency of the EGFR ECD mutation in each genomic DNA sample was assessed by droplet digital PCR (ddPCR) using a QX200™ Droplet Digital™ PCR system (Bio-Rad). A Custom TaqMan® SNP Genotyping Assay kit containing ddPCR primers specific for the EGFR S492R and G465R mutations and corresponding wild type was obtained from Life Technologies (Catalog#4331349; Assay IDs: AHN1YEK, EGFRS492R; Assay IDs: AHPAWKS, EGFRG465R). The ddPCR assay was performed according to the manufacturer's instructions using 20 ng of genomic DNA per reaction and the allelic frequency was calculated as the percentage of EGFR mutant DNA alleles to total (EGFR mutant plus wild type) DNA alleles.

### NanoBRET Assay

HEK293 cells were transiently transfected with FuGENE® HD Transfection Reagent (Promega cat#E2311) to allow expression of the EGFR-NanoLuc mutants. Transfected cells were then treated with increasing doses (from 0 to 100 μg/ml) of drugs and HaloTag-EGF (tracer) at a concentration of 18 ng/ml for 30 minutes. Then the NanoBRET™ Nano-Glo® Substrate is added and the plates are analyzed with the GloMax®-Multi Microplate Multimode Reader. To calculate the raw NanoBRET™ ratio values, the acceptor emission value (610 nm) is divided by the donor emission value (450 nm) for each sample.

### Cell Viability Assay

Cetuximab and panitumumab were obtained from the Pharmacy at Niguarda Ca' Granda Hospital, Milan, Italy. MM-151 was obtained from Merrimack Pharmaceuticals. Cell lines were seeded in 100 μL medium at the following densities (2×10³ for LIM1215, HCA-46, NCIH508, and OXCO-2, 3×10³ for CCK81) in 96-well culture plates. The following day, 100 μL of serial dilutions of each drug (cetuximab, panitumumab, MM-151) were added to cells in serum-free medium to achieve final concentrations between 100 and 0 μg/ml. Plates were incubated at 37° C. in 5% CO₂ for 6 days, after which cell viability was assessed by ATP content using the CellTiter-Glo® Luminescent Assay (Promega). Measurements were recorded using Victor-X4 plate reader (PerkinElmer). Treated wells are normalized to untreated.

Experiments with the LIM1215 (#2) cell line and cell lines derived from it use a modified method. Cetuximab was obtained from Myoderm and the Sym004 combination (Sym992+Sym1024) antibodies were expressed and purified using sequence information from the patent literature, and MM-151 was obtained from Merrimack Pharmaceuticals. Cell lines were seeded in 100 μL RPMI medium (Life Technologies) supplemented with 10% fetal bovine serum at a density of 5×10³ cells/well in 96-well culture plates. The following day, 100 μL of serial dilutions of each drug (cetuximab, Sym004, MM-151) were added to cells in RPMI medium supplemented with 8 nM EGF ligand (Preprotech) to achieve final concentrations between 1000 and 1 nM. Plates were incubated at 37° C. in 5% CO₂ for 3 days, after which cell viability was assessed by ATP content using the CellTiter-Glo® Luminescent Assay (Promega). Measurements were recorded using Envision plate reader (PerkinElmer). Treated wells are normalized to wells treated with 8 nM EGF ligand alone.

### Cell Growth Rate Assay

Cetuximab was obtained from Myoderm, the Sym004 combination (Sym992+Sym1024) antibodies were expressed and purified using sequence information from the patent literature, and MM-151 was obtained from Merrimack Pharmaceuticals. Cell lines were seeded in 100 μL RPMI medium (Life Technologies) supplemented with 10% fetal bovine serum at a density of 5×10³ cells/well in 96-well culture plates. The following day, seeding media was exchanged for 300 μL of RPMI treatment media which contained 2% fetal bovine serum and serial dilutions of each drug (to achieve final concentrations between 1000 and 0.15 nM) and supplemented with either no ligand, 8 nM EGF ligand (Peprotech), or 8 nM AREG ligand (Peprotech). Plates were immediately moved to an IncuCyte ZOOM live-cell imager and phase contrast images were acquired at two-hour intervals for a period of 72 hours. To calculate growth rate, confluence data (percent of well surface covered by cells) was natural log-transformed, and the slopes of the resulting lines were determined via linear regression. To exclude growth saturation at high confluence, only measurements between 0-70% confluence were used. Growth rates were normalized to control (no drug) conditions for each cell line.

### Phospho-ERK Cell Signaling Assay

Cell lines were seeded in 100 μL RPMI medium (Life Technologies) supplemented with 10% fetal bovine serum at a density of 3.5×10⁴ cells/well in 96-well culture plates (Costar; Catalog#3997). The following day, seeding media was exchanged for 100 μL RPMI medium supplemented with 1% fetal bovine serum. The following day, cells were treated for two hours with 100 μL of media containing 1% fetal bovine serum and serial dilutions of each drug (to achieve final concentrations between 1000 and 0.15 nM). Cells were then stimulated for 10 minutes with 50 μL RPMI containing 1% fetal bovine serum and either no ligand, EGF ligand (Peprotech), or AREG ligand (Peprotech). After addition, the final concentration of the ligand was 8 nM. Cells were then washed with 150 μL cold PBS and lysed in 54 μL MPER buffer+150 mM NaCl+phosphatase inhibitors (Roche; Catalog#04906837001 and 4693124001). Lysates were frozen for 15 minutes at −80 C and then thawed on ice for 30 minutes.

Phospho-ERK was measured by sandwich ELISA. 96-well blank MSD plates (Meso Scale Discovery; Catalog# L15XA-3) were coated with 25 μL 2× capture antibody (Cell Signaling Technologies; Catalog# R92TC-1) diluted from 100X in PBS (1:50), and incubated at room temperature overnight. The following day, plates were washed three times with 100 μL/well PCST (0.05% Tween-20) on a BIOTEK plate washer. Plates were subsequently blocked for one hour at room temperature with 150 μL MSD block (1% in PBS, 0.2 μm filtered). The plates were washed three times with 100 μL/well PBST (0.05% Tween-20) on a BIOTEK plate washer. Cell lysates (neat) or phospho-ERK standards (R&D Systems; Catalog# DYC1018BE) were added at 25 μL/well and incubated overnight at 4° C. The plates were washed three times with 100 μL/well PBST (0.05% Tween-20) on a BIOTEK plate washer. The detection antibody (Cell Signaling Technologies; Catalog# R92TC-1) was diluted from 100× to 2× in 1% MSD block solution (1:50), added at 25 μL/well, and incubated for two hours at room temperature. The plates were washed three times with 100 μL/well PBST (0.05% Tween-20) on a BIOTEK plate washer. The secondary detection anti-mouse IgG with Sulfo-tag (Meso Scale Discovery; Catalog# R32AC-5) was diluted 1:1000 in 1% MSD block solution, added at 25 μL/well, and incubated for one hour at room temperature on an orbital shaker (300 rpm). The plates were washed three times with 100 μL/well PBST (0.05% Tween-20) on a BIOTEK plate washer. MSD reading buffer with surfactant (Meso Scale Discovery; Catalog# R92TC-1) was diluted from 4× to 2× in ddH20, and added at 150 μL/well. Plates were read on an MSD SECTOR Imager 2400 (Meso Scale Discovery). The results were analyzed by subtraction of the background signal, regression to the recombinant ERK standard, and back-calculation to correct for dilution factors. Replicate samples were averaged and the standard deviation calculated.

### DNA Constructs and Mutagenesis

The NanoLuc®-EGFR WT vector was purchased from Promega Corporation, while the pLX301-EGFR WT construct was a generous gift from Dr. C. Sun and Prof R. Bernards (NKI, Amsterdam, the Netherlands).

EGFR mutants containing the 6 point mutations (R451C, S464L, G465R, K467T, I491M, and S492R) were constructed using the QuikChange II site-directed mutagenesis kits from Agilent Technologies Using the WT corresponding plasmid as the template DNA. The presence of mutations was confirmed by DNA sequencing.

### Immunoblot Analysis

Prior to biochemical analysis, parental cells were grown in their specific medium supplemented with 10% FBS, KI cells in 5% FBS medium. Total cellular proteins were extracted by solubilizing the cells in cold EB buffer (50 mM Hepes pH 7.4, 150 mM NaCl, 1% Triton X-100, 10% glycerol, 5 mM EDTA, 2 mM EGTA; all reagents were from Sigma-Aldrich, except for Triton X-100 from Fluka) in the presence of 1 mM Sodium orthovanadate, 100 mM Sodium fluoride and a mixture of protease inhibitors (pepstatin, leupeptin, aprotinin and STI). Extracts were clarified by centrifugation, and protein concentration was determined using a BCA protein assay reagent kit (Thermo). Western blot detection was performed with enhanced chemiluminescence system (GE Healthcare) and peroxidase conjugated secondary antibodies (Amersham). The following primary antibodies were used for western blotting (all from Cell Signaling Technology, except where indicated): anti-phospho p44/42 ERK (thr202/tyr204); anti-p44/42 ERK; anti-phospho-AKT (Ser473), anti-AKT; anti-phospho EGFR (tyr1068); anti-EGFR (clone13G8, Enzo Life Sciences); anti-vinculin (Sigma-Aldrich). The following day, after 1 h of incubation with appropriate secondary antibody, signal was developed using the ECL system (Amersham Biosciences).

Experiments with the LIM1215 (#2) cell line and cell lines derived from it use a modified method. Cells were seeded in 2 mL RPMI medium supplemented with 10% fetal bovine serum at a density of 5×10⁵ in 6-well plates. The following day, seeding media was exchanged for 1 mL of RPMI medium supplemented with 1% fetal bovine serum. The following day, cells were treated for 24 hours with 111 μL of media containing 1% fetal bovine serum and drug (to achieve a final concentration of 100 nM). The following day, cells were stimulated for 10 minutes with 123 μL RPMI containing 1% fetal bovine serum and either no ligand, EGF ligand (Peprotech), or AREG ligand (Peprotech). After addition, the final concentration of the ligand was 8 nM. Cells were then washed with 1 mL cold PBS and lysed in 100 μL MPER buffer+150 mM NaCl+phosphatase inhibitors (Roche; Catalog#04906837001 and 4693124001). Extracts were frozen at −80 C. At a later date, extracts were clarified by centrifugation, and protein concentration was determined using a BCA protein assay reagent kit (Thermo Fisher).

Samples were prepared for Western blot analysis as follows. 40 μg of protein was diluted in loading buffer (Bio-Rad) supplemented with 10% b-Mercaptoethanol and MPER buffer to a final volume of 30 μL. Samples were boiled at 90 C for 5 minutes then vortexed and centrifuged. 25 μL sample was loaded into wells of 4-12% Criterion XT Bis-Tris Gel (Bio-Rad; Catalog#3450124) and run according to manufacturers instructions at 180V for 90 minutes. The gel was then incubated in 2× NuPAGE Transfer Buffer (ThermoFisher) for 20 minutes on a shaker and then transferred onto a nitrocellulose membrane with an iBlot transfer system (ThermoFisher). The membrane was blocked for 1 hour at room temperature on a shaker in Odyssey Blocking Buffer (Li-cor; Catalog#927-40000) and then incubated overnight in primary antibody diluted in blocking buffer at 4 C on a shaker (pEGFR: Cell Signaling Technologies, Catalog#3777; total EGFR: Cell Signaling Technologies, Catalog #4267; actin house-keeping control: Sigma Catalog# A1978). On the following day, the membrane was washed three times with TBS-T solution for 5 minutes each and then incubated with secondary antibodies in blocking buffer at room temperature for one hour on a shaker. Secondary antibodies used were IRDye 800CW goat anti-rabbit IgG (H+L) (Li-cor, Catalog#926-32211) and IRDye 680RD goat anti-mouse IgG (H+L) (Li-cor, Catalog#926-68070). Membranes were then washed three times with TBS-T solution for 5 minutes each and then once with PBS. Membranes were then scanned using an Odyssey Imager (Li-Cor).

### Assessment of Antibody Binding by Flow Cytometry

Cells were transferred to a U-bottom 96-well plate (Falcon; catalog #353077) at a density of 1×10⁵ cells/well in 45 μL FACS buffer (phosphate-buffered serum, pH 7.4, supplemented with 1% fetal bovine serum and 0.1% sodium azide) and 5 μL of Human TruStain FcX™ solution (BioLegend, Inc; catalog #422302). Cells were incubated for 10 minutes on ice (4° C.). Then, antibody conjugated with Alexa-Fluor® 488 dye (ThermoFisher) and diluted in FACS buffer was added for 1 hour at a final concentration of 0.1 μM. To confirm binding specificity, half of the wells were first incubated for 1 hour with unlabeled antibody (at a final concentration of 5 μM). Cells were washed twice with FACS buffer on ice (4° C.) and resuspended in TO-PRO®-3 Iodide (642/661) solution (ThermoFisher; catalog #T3605) diluted 1:10000 in FACS buffer. Cells were then loaded onto a FACSCalibur™ (BD Biosciences) for analysis. Live cells were selected (TO-PRO®-3 negative) and the intensity of Alexa-Fluor® 488 measured. FlowJo® software (FlowJo, LLC) was used to calculate the mean fluorescence intensity for each sample.

### Murine Xenograft Studies

Female nu/nu mice (NU-Foxn1^{nu}; Charles River Labs), aged 4-5 weeks, weighing 16±0.5 g, were inoculated as indicated with 0.2 mL of LIM1215 (#2) EGFR S492R, 0.2 mL of LIM1215 (#2) EGFR S492R KI, or 0.2 mL of LIM1215 (#2) cell suspension in PBS containing growth factor-reduced Matrigel (BD Biosciences) at a density of 5×10⁶ cells per mouse. Once tumors had reached approximately 300 mm³ in volume, mice were randomized into treatment groups to receive PBS (vehicle control) or drug. Treatments were administered by IP injection. Tumor dimensions were measured three times each week by calipers and the tumor volumes were calculated using the formula: π/6×L×W², where L and W, respectively, represent the larger and smaller tumor diameter.

The administration schedule for antibodies is determined based upon their pharmacokinetics. Antibodies that do not cross-react with murine EGFR (25E, P3X, cetuximab) were observed to have nearly-equivalent half-life values of approximately 4-5 days in mice and are administered on a Q7d schedule (e.g. Monday). Antibodies that cross-react with murine EGFR (P1X, P2X) were observed to have more rapid clearance due to target-mediated drug disposition and are administered on a 3× weekly schedule (e.g. Monday, Wednesday, Friday).

A three-antibody mixture consisting of 25E, P2X, and P3X antibodies is utilized as a MM-151 tool compound for tumor growth experiments in mice. The antibody 25E substitutes for P1X because it does not engage murine EGFR and thus allows for a weekly administration schedule that matches the cetuximab comparator and should better reflect the pharmacokinetics in a human patient. P1X is an affinity matured variant of 25E (11 vs. 145 pmol/L) and the antibodies engage overlapping epitopes on EGFR. The doses of the three antibodies in the MM-151 tool compound were calculated using computational simulations of a pharmacokinetics model for each antibody to result in summed serum exposure equivalent to that of the corresponding dose of cetuximab.

Analysis of EGFR ECD Mutations in Circulating Cell-Free DNA from Human Patients

A Phase 1 study of MM-151 in patients with refractory advanced solid tumors was conducted to evaluate safety and establish a maximum tolerated dose of MM-151 as a monotherapy or in combination with irinotecan (Protocol MM-151-01-01-01, NCT#01520389). As part of this protocol, blood and tumor tissue samples were collected and written informed consent was obtained from all patients for exploratory biomarker analysis to further characterize and correlate possible biomarkers that may help to predict or evaluate MM-151 response and/or safety. The study was reviewed and approved by the institutional review board at each site, according to local guidelines. The study was designed to evaluate escalating doses of MM-151 at various schedules. Patients were treated until progressive disease (assessed by radiological scans every eight weeks from the date of first dose), intolerable toxicity or other reason for discontinuation as assessed by the investigator. Serum samples were collected at protocol-defined time points to support biomarker analyses. At each collection time point, 9.5 mL blood was collected in a red top tube without additive and allowed to clot for 15-30 minutes at room temperature. The sample was then centrifuged at 3000 rpm for 10-15 minutes to separate cells from serum. The serum was split into two equal aliquots and placed into −80° C. storage until shipment to a central storage facility.

Circulating cell-free DNA (cfDNA) was isolated from serum using the QIAamp Circulating Nucleic Acid Kit (QIAGEN) according to the manufacturer's instructions. 6 μl of ctDNA were then used as template for each qPCR reaction for GE/ml measurement. All samples were analyzed in triplicate. PCR reactions were performed using 10 μl final volume containing 5 μl GoTaq® qPCR Master Mix, 2× with CXR Reference Dye) (Promega) and LINE-1 [1.5 μmol] forward and reverse primers. Isolated cfDNA was amplified using ddPCR Supermix for Probes (Bio-Rad) using primers specific to each EGFR ECD mutation. Droplet digital PCR (ddPCR) was performed according to manufacturer's protocol and the results reported as percentage or fractional abundance of mutant DNA alleles to total (mutant plus wild type) DNA alleles. Eight to 10 μl of DNA template was added to 10 μl of ddPCR Supermix for Probes (Bio-Rad) and 2 μl of the primer/probe mixture. This 20 μl sample was added to 70 μl of Droplet Generation Oil for Probes (Bio-Rad) and used for droplet generation. Droplets were then thermal cycled with the following conditions: 5 minutes at 95° C., 40 cycles of 94° C. for 30 seconds, 55° C. for 1 minute followed by 98° C. for 10 minutes (ramp rate 2° C./sec). Samples were then transferred to a QX200 Droplet Reader (Bio-Rad) for fluorescent measurement of FAM and HEX probes. Gating was performed on the basis of positive and negative controls, and mutant populations were identified. Fractional abundances of the mutant DNA in the wild type DNA background were calculated for each sample using QuantaSoft software (Bio-Rad). Multiple replicates (minimum of three) were performed for each sample. ddPCR analysis of normal control gDNA from cell lines and no DNA template (water) controls were performed in parallel with all samples, including also multiple replicates as a contamination-free control.

### Example 1

### EGFR Extracellular Domain (ECD) Mutants have Reduced Ability to Bind Ligands and Monoclonal Anti-EGFR Antibody Preparations

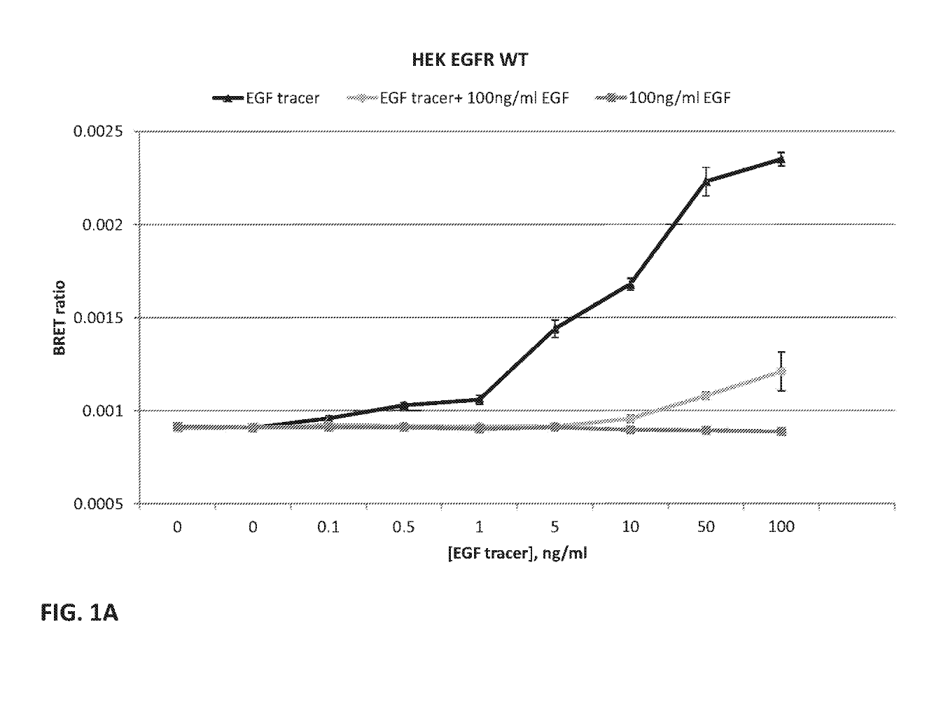

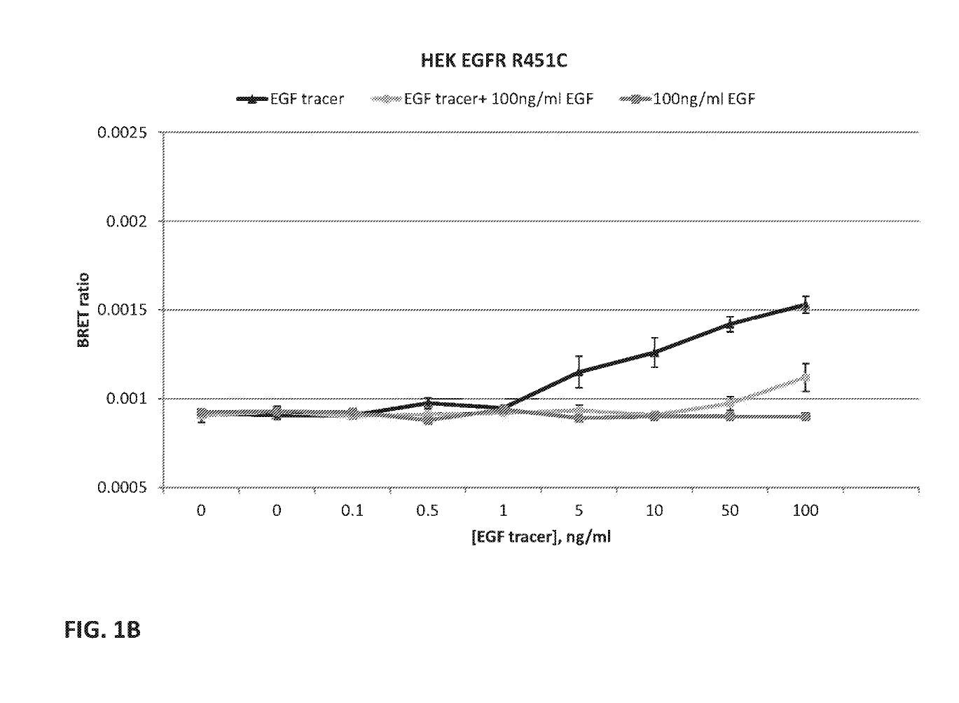

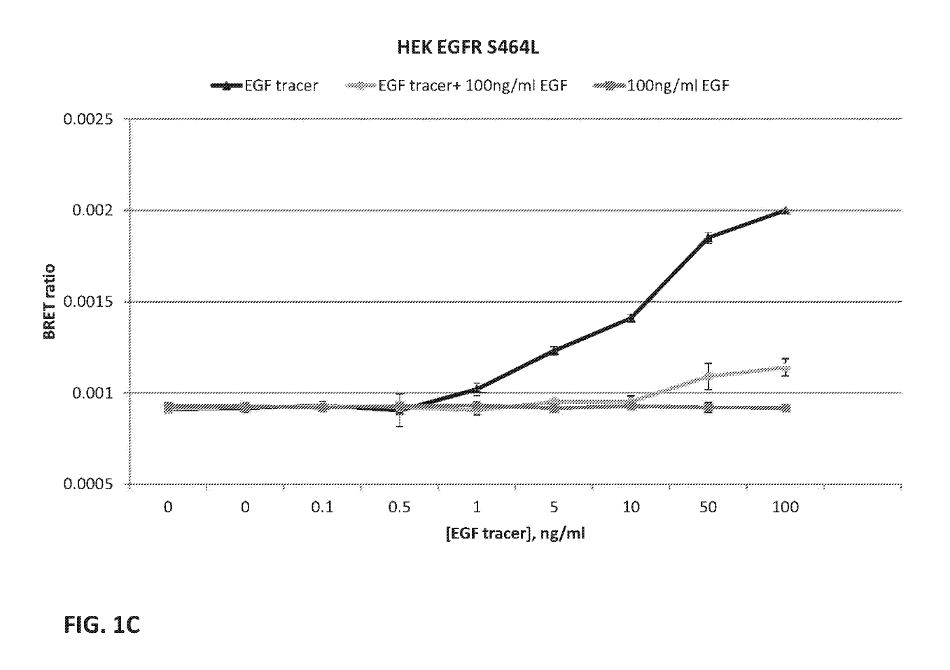

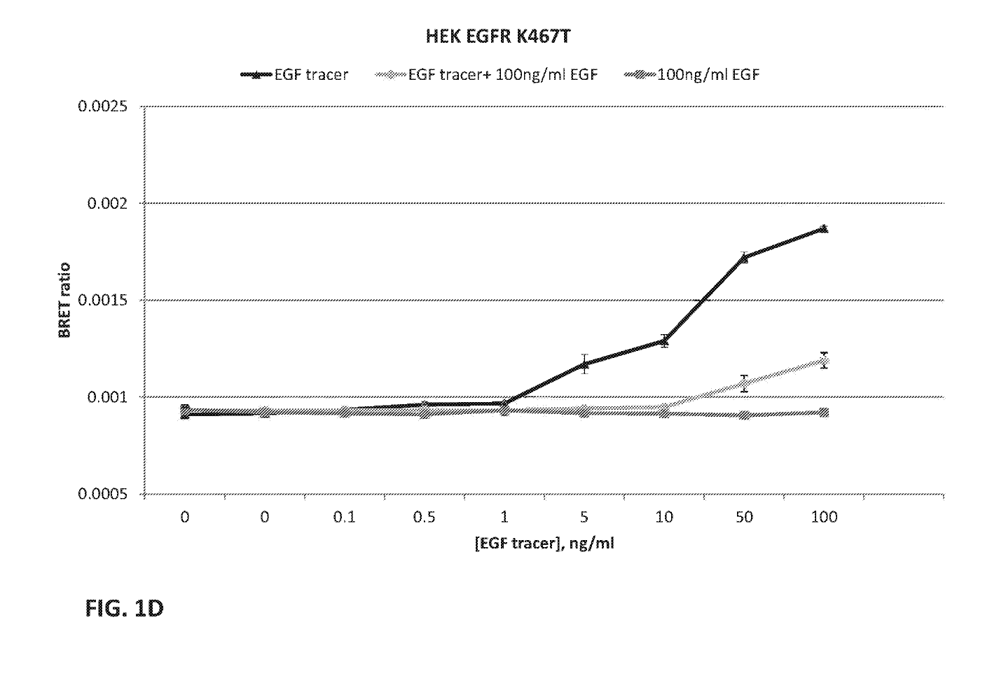

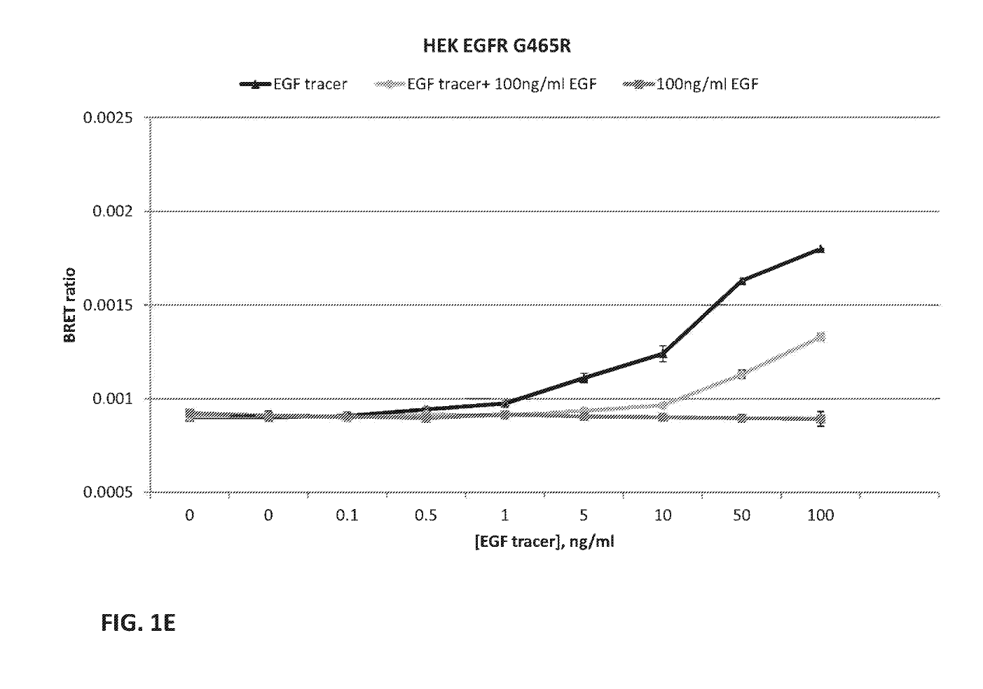

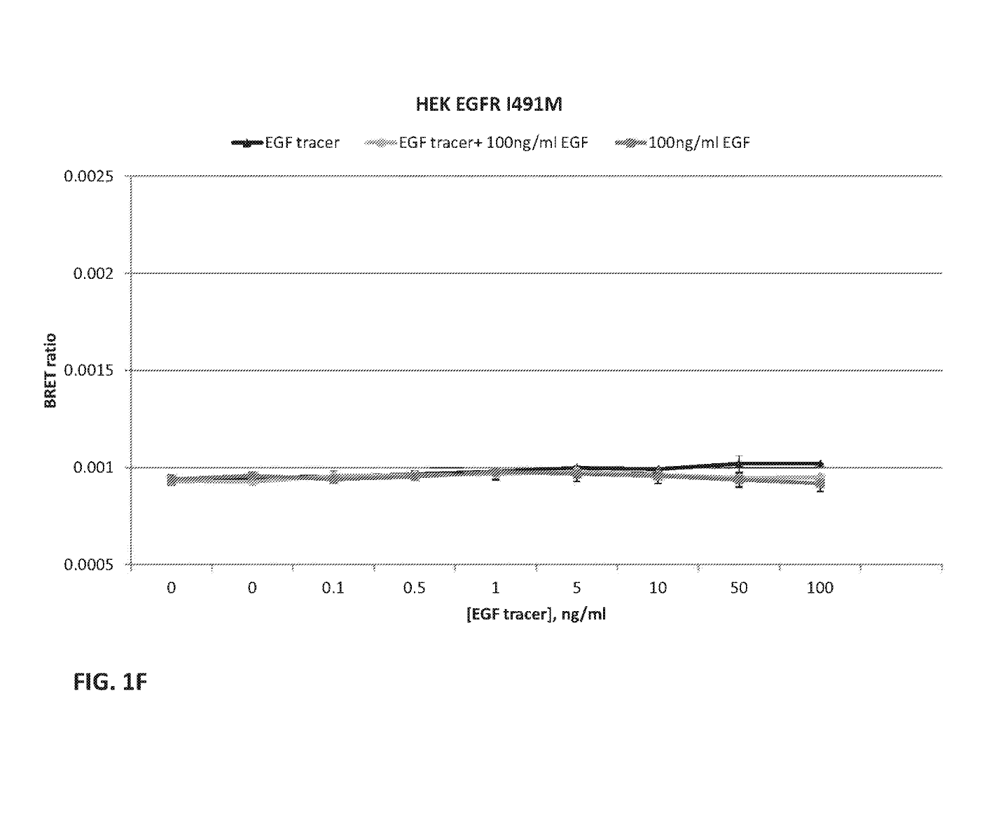

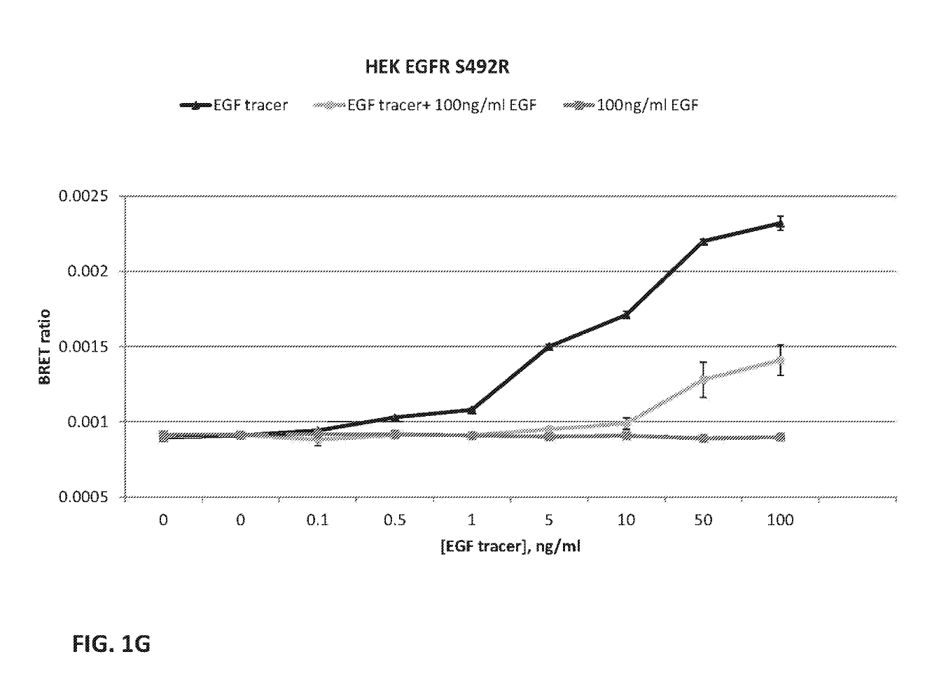

Bioluminescence Resonance Energy Transfer (BRET) is used to quantitatively measure the interaction between proteins in live cells. The NanoBRET™ system (Promega) is used with NanoLuc® luciferase as the bioluminescent energy donor and fluorescent-labeled HaloTag® protein as the energy acceptor. To determine whether EGF ligand can bind EGFR mutants, an EGFR Tracer dose response assay was performed. HEK 293 cells were transfected with plasmids expressing the indicated NanoLuc-EGFR mutants (FIG. 1) and then treated with increasing dose of EGF tracer (HaloTag-EGF) in the presence (light grey line) or absence (black line) of an excess amount of unabeled EGF to assess that EGF tracer can effectively bind to the NanoLuc-EGFR. NanoBRET™ Nano-Glo® Substrate was added and the plates were analyzed with the GloMax®-Multi Microplate Multimode Reader. To calculate the raw NanoBRET™ ratio values, the acceptor emission value (618 nm) was divided by the donor emission value (460 nm) for each sample.

As shown in FIG. 1, protein-protein interactions between ligand (EGF) and EGFR wild-type (FIG. 1A) and extracellular domain mutants were measured, the mutants including EGFR R451C (FIG. 1B), S464L (FIG. 1C), K467T (FIG. 1D), G465R (FIG. 1E), I491M (FIG. 1F), and S492R (FIG. 1G). All EGFR mutants tested, except for EGFR I491M, were able to bind the EGF tracer in this EGFR tracer dose response assay.

### Example 2

### MM-151 is Able to Bind all EGFR Ectodomain Mutants in a Drug Displacement Assay

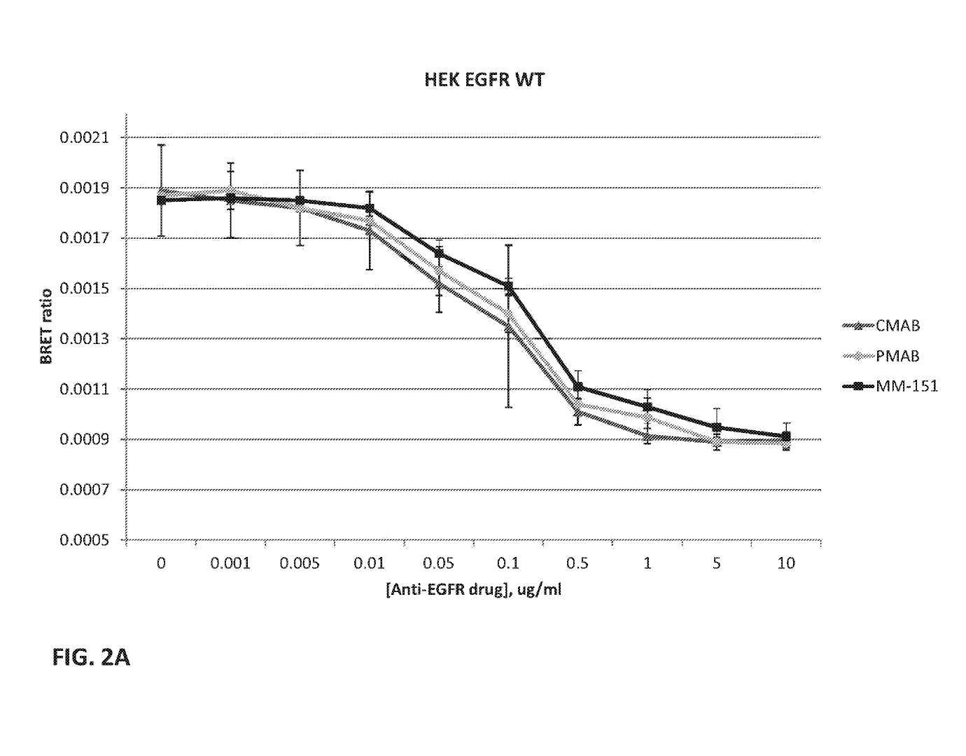

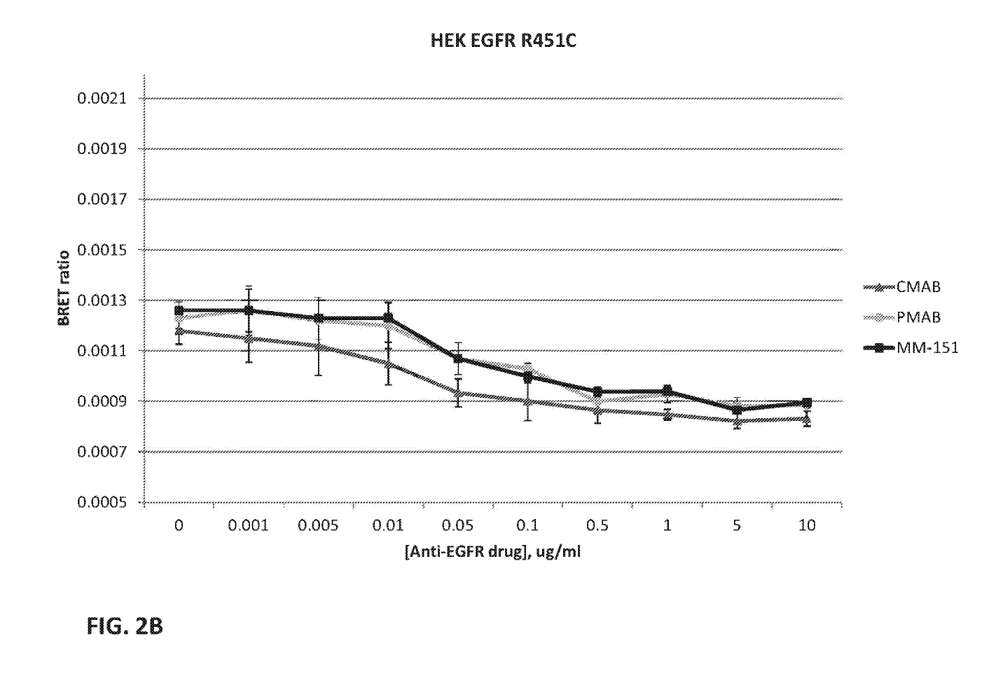

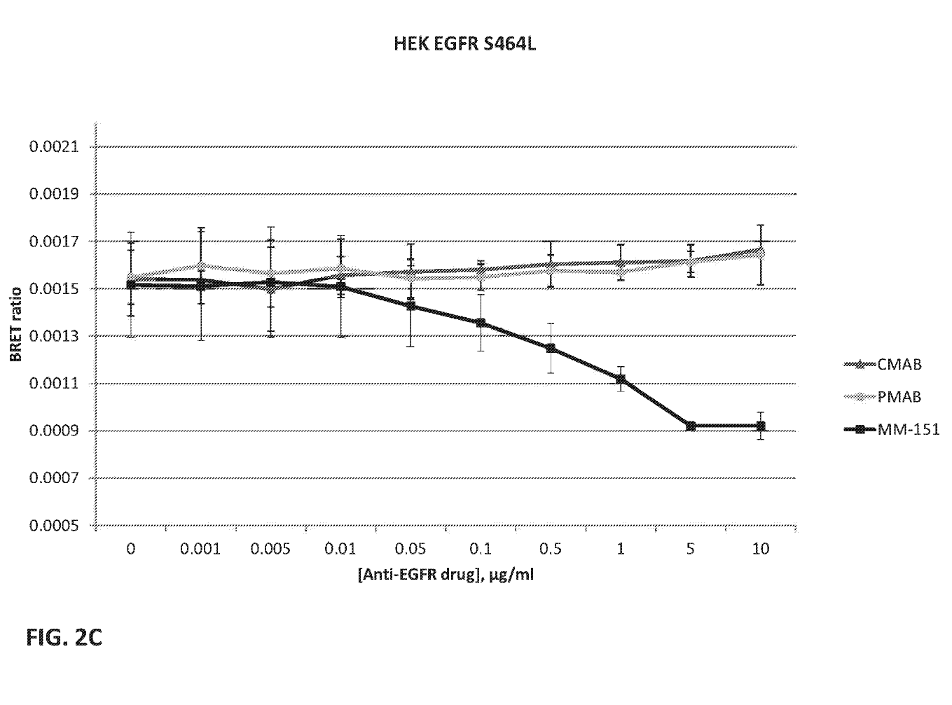

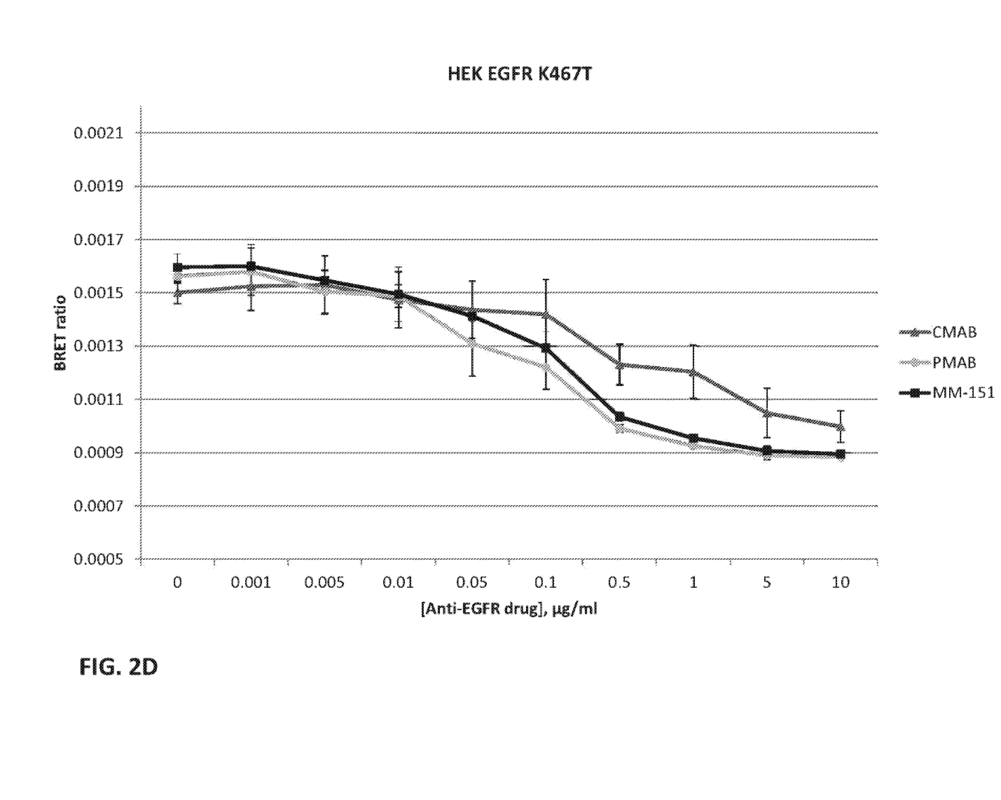

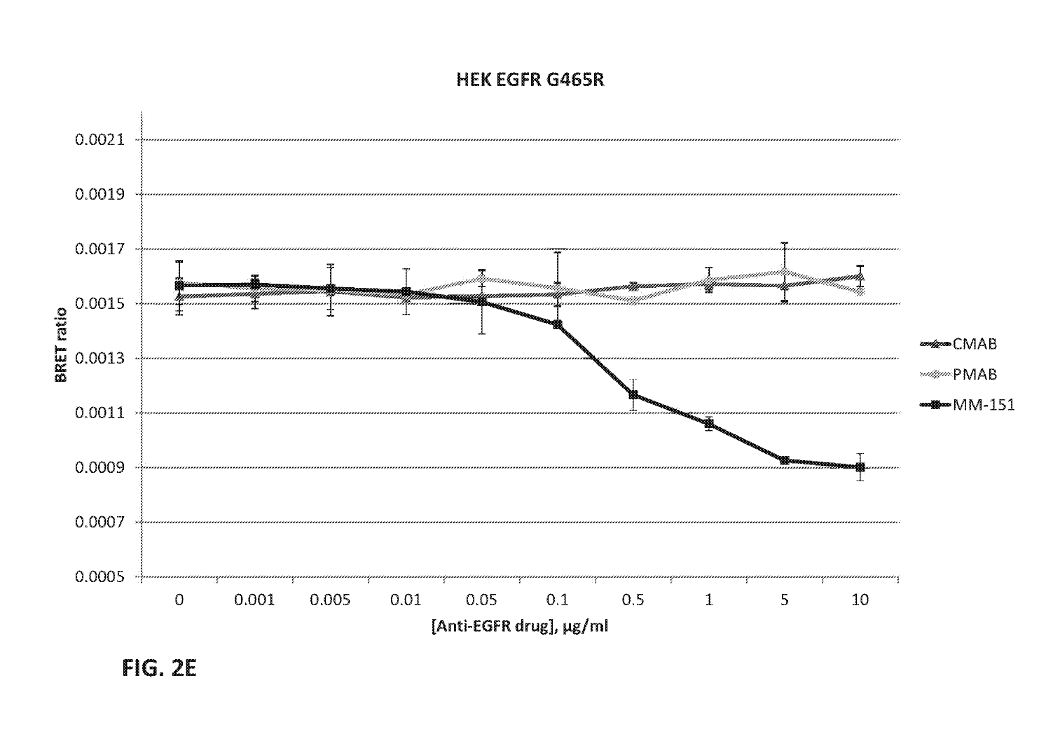

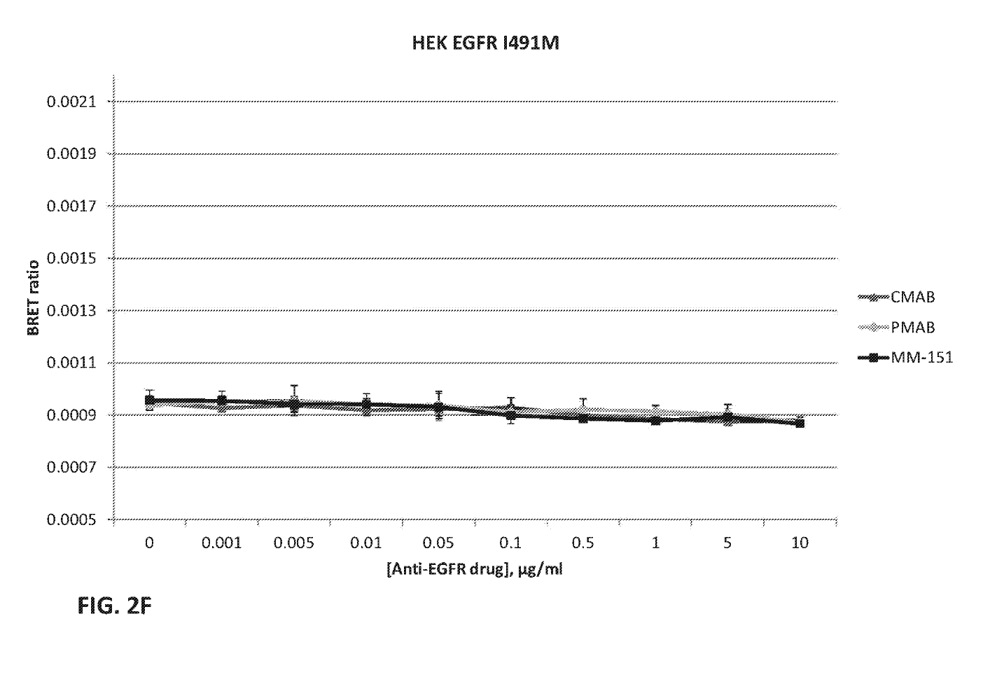

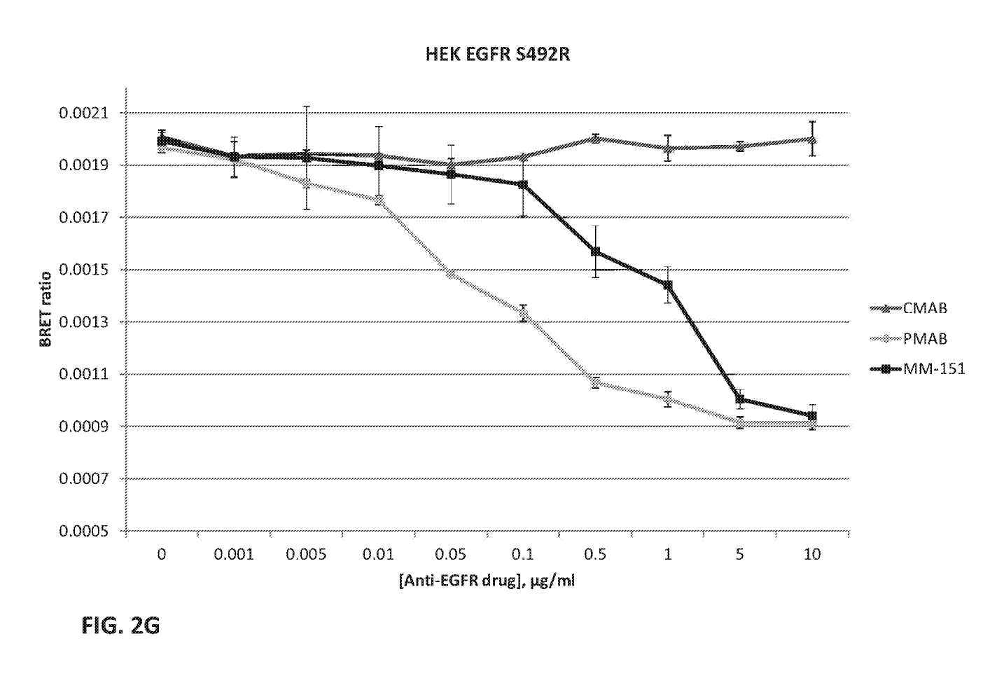

In order to evaluate the interaction between EGFR mutants and anti-EGFR therapeutics a drug displacement assay was performed. HEK 293 cells were transfected with plasmids expressing wild-type EGFR (FIG. 2A) and the NanoLuc-EGFR mutants EGFR R451C (FIG. 2B), S464L (FIG. 2C), K467T (FIG. 2D), G465R (FIG. 2E), I491M (FIG. 2F), and S492R (FIG. 2G). The mutants were then treated with increasing doses (from 0 to 100 μg/ml) of the anti-EGFR drugs cetuximab (“CMAB”, grey triangles), panitumumab (“PMAB”, light grey circles) or MM-151 (black squares) and HaloTag-EGF (tracer) at a concentration of 18 ng/ml for 30 minutes. NanoBRET™ Nano-Glo® Substrate was added and the plates were analyzed with the GloMax®-Multi Microplate Multimode Reader. To calculate the raw NanoBRET™ ratio values, the acceptor emission value (618 nm) was divided by the donor emission value (460 nm) for each sample.

As shown in FIG. 2, MM-151 effectively binds to all of the EGFR mutants tested, whereas cetuximab did not bind to EGFR-S464L, EGFR-G465R, or EGFR-S492R, and panitumumab did not effectively bind to EGFR-S464L or EGFR-G465R. Thus, only MM-151 out of the drugs tested will bind all the mutant EGFR extracellular domains.

### Example 3

### LIM1215 Overexpressing EGFR Ectodomain Mutants and Resistant to Cetuximab or Both to Cetuximab and Panitumumab are Sensitive to MM-151

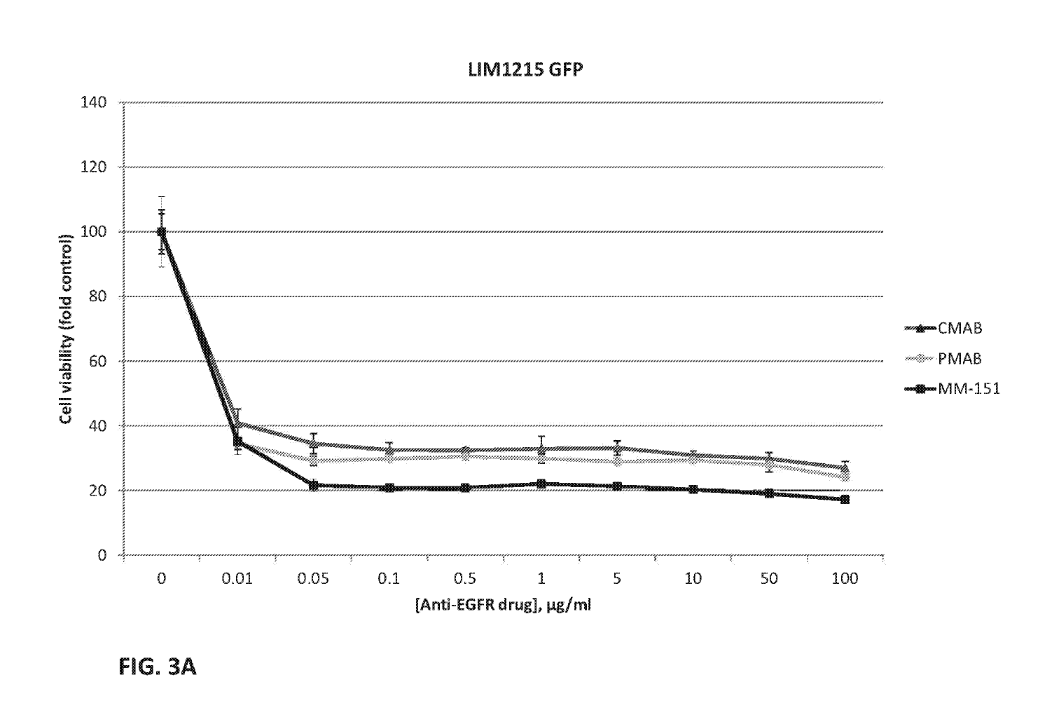

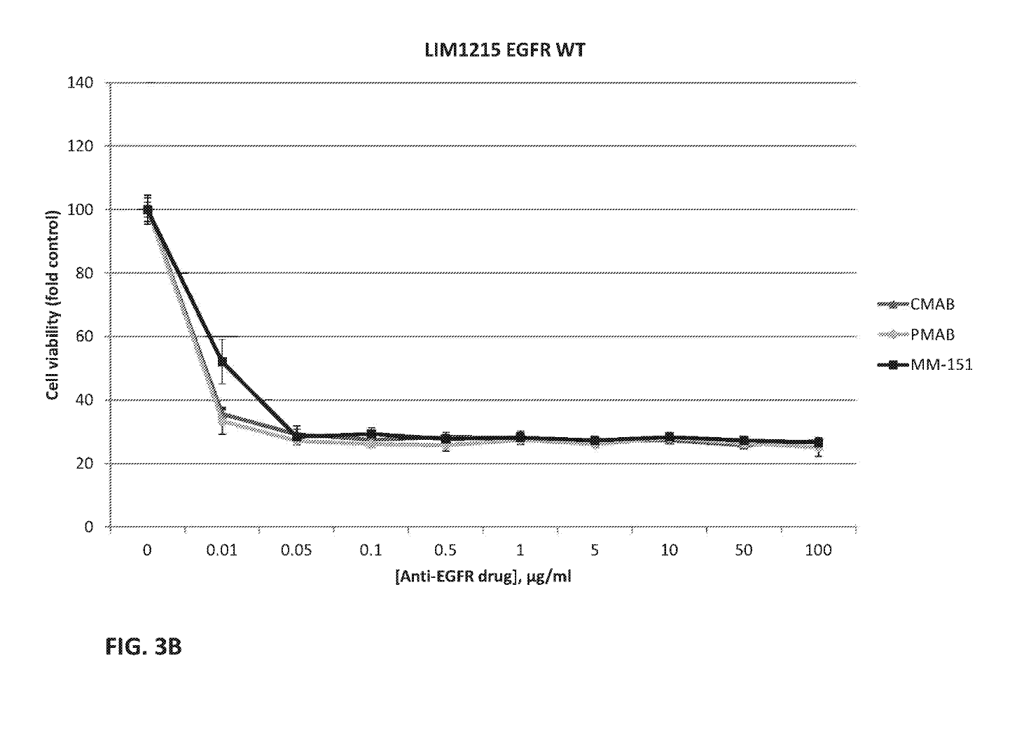

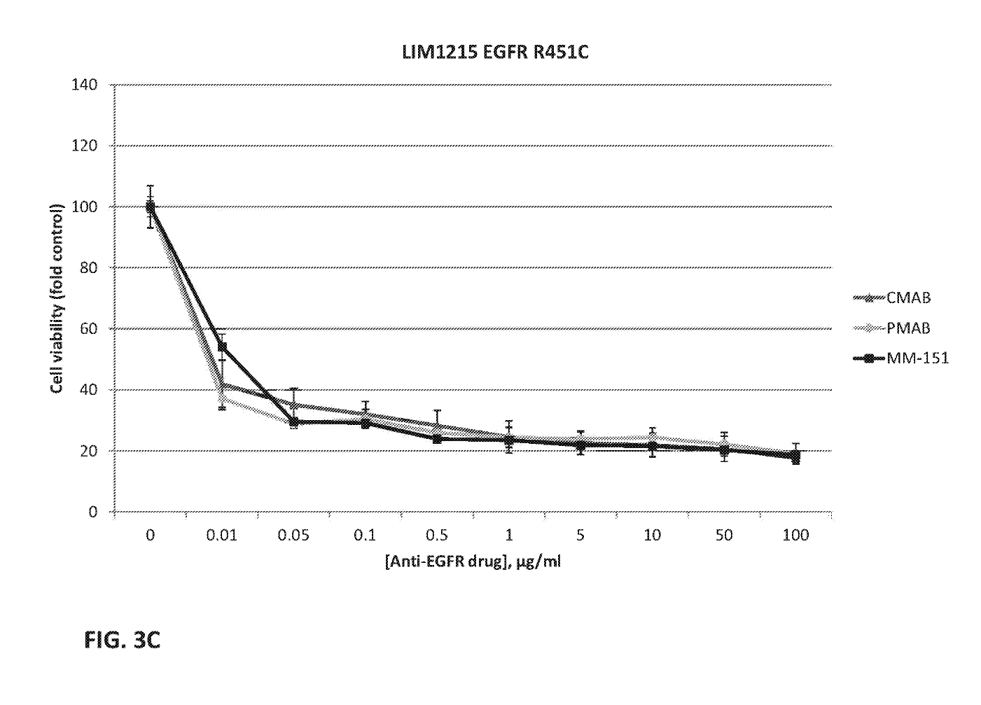

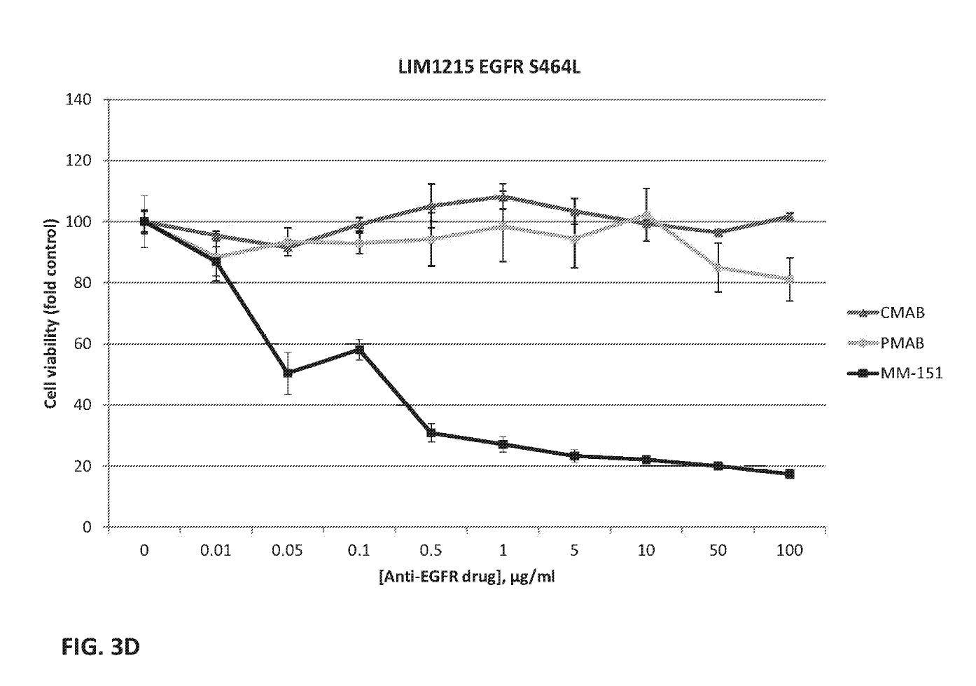

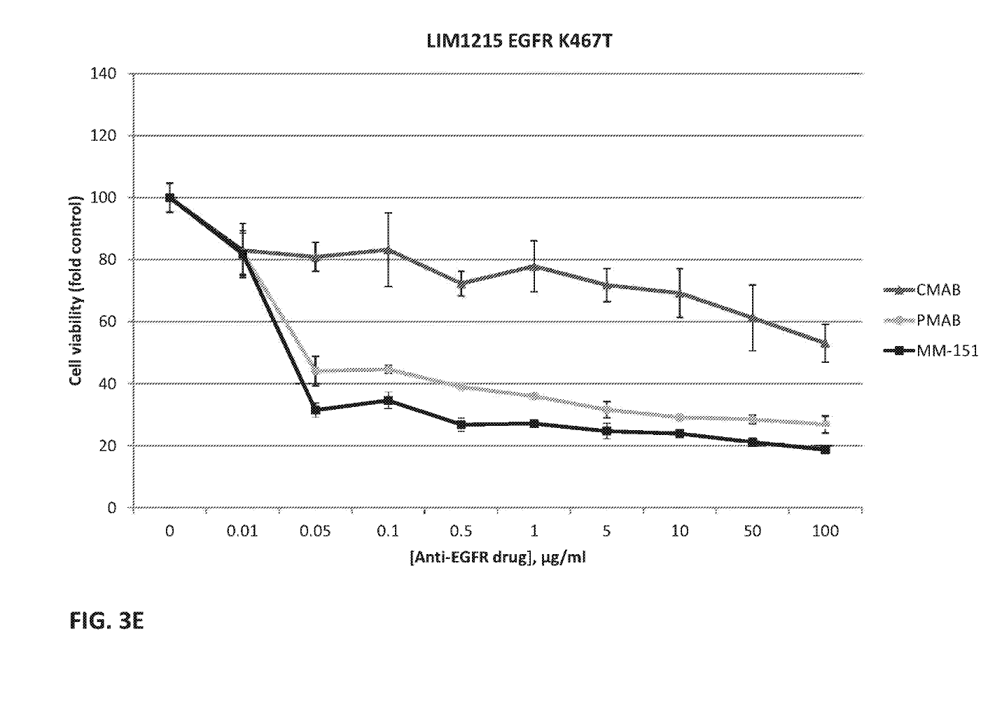

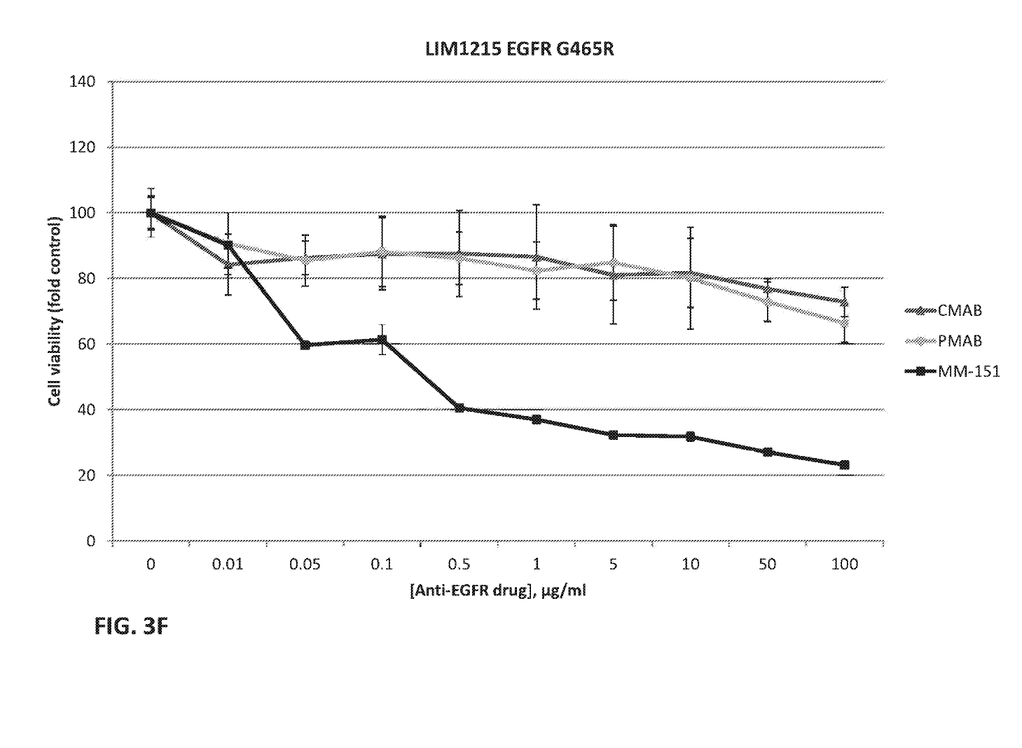

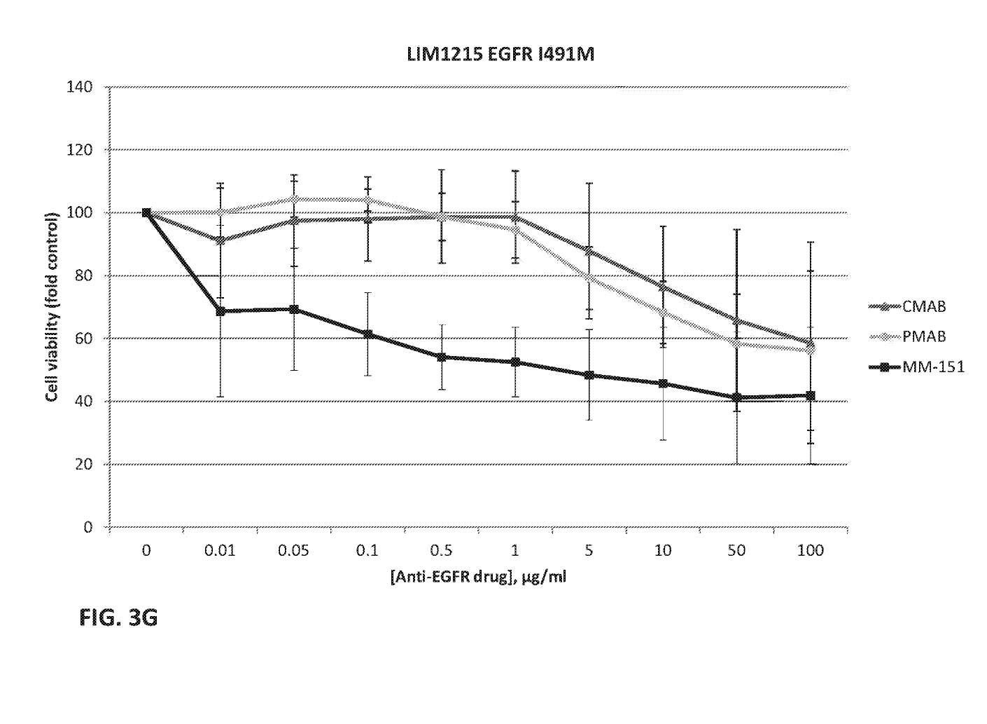

LIM1215 cells (human colorectal carcinoma, CellBank Australia Cat. # CBA-0161) were infected with lentivirus expressing the indicated EGFR ectodomain mutants and controls, including GFP control (FIG. 3A), EGFR wild-type (FIG. 3B), EGFR R451C (FIG. 3C), S464L (FIG. 3D), K467T (FIG. 3E), G465R (FIG. 3F), I491M (FIG. 3G), and S492R (FIG. 3H). Selected cells were treated for 6 days with increasing concentrations of cetuximab (“CMAB”, dark grey line), panitumumab (“PMAB”, light grey line) and MM-151 (black line). Cell viability was measured by the adenosine triphosphate (ATP) assay. As shown in FIG. 3, cetuximab was not able to inhibit growth of cells expressing EGFR with the mutations S464L, G465R, K467T, I491M, or S492R; and panitumumab was not able to inhibit growth of cells expressing EGFR with the mutations S464L, G465R, or I491M. By contrast, MM-151 was able to inhibit cell growth in all mutants and controls tested.

### Example 4

### MM-151 Blocks EGFR Downstream Signaling in LIM1215-Overexpressing EGFR Ectodomain Mutants that are Resistant to Cetuximab or to Both Cetuximab and Panitumumab

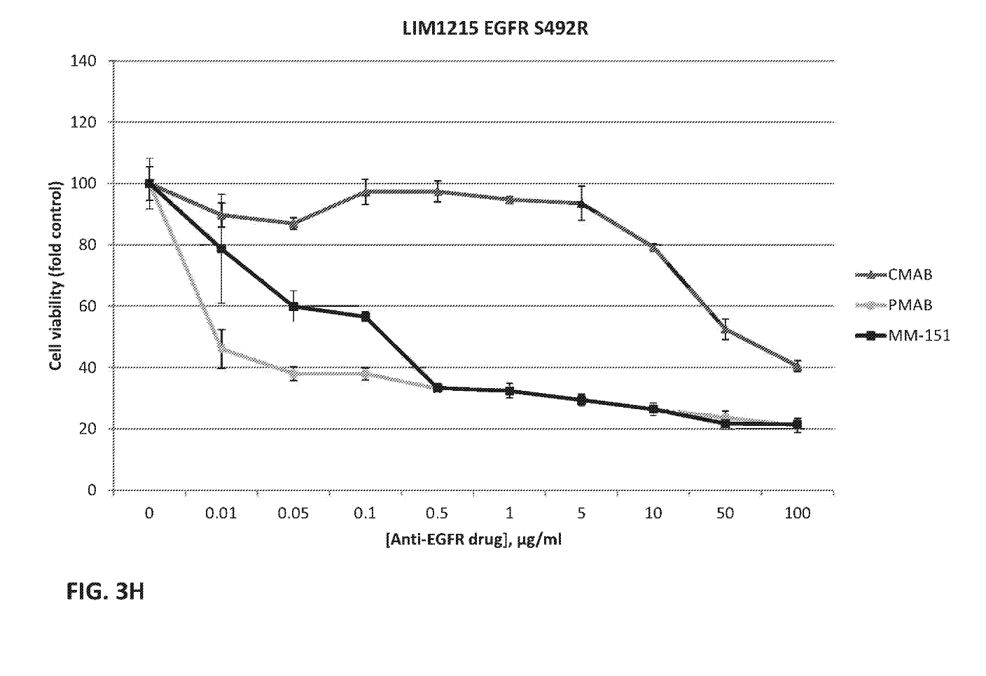

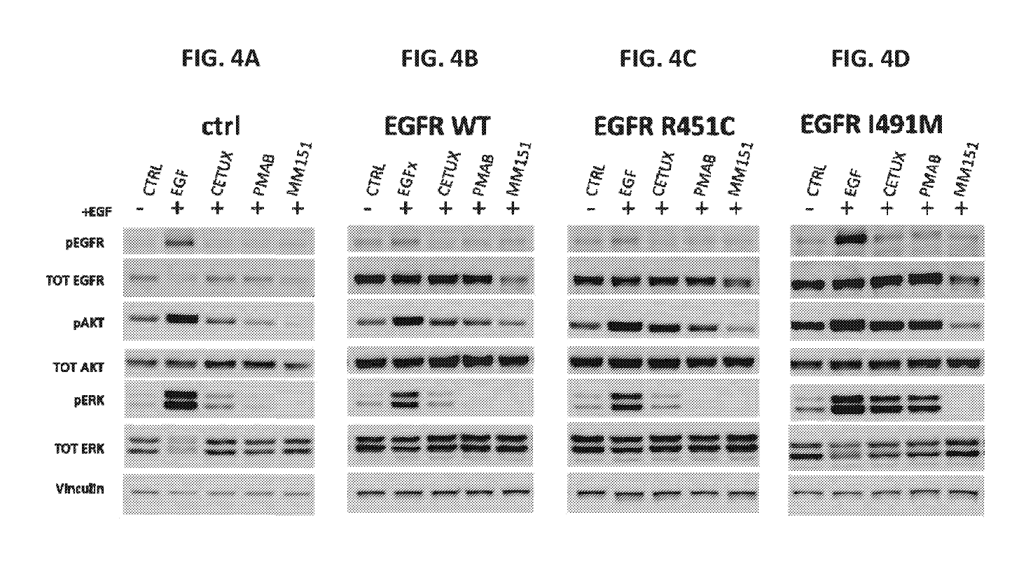

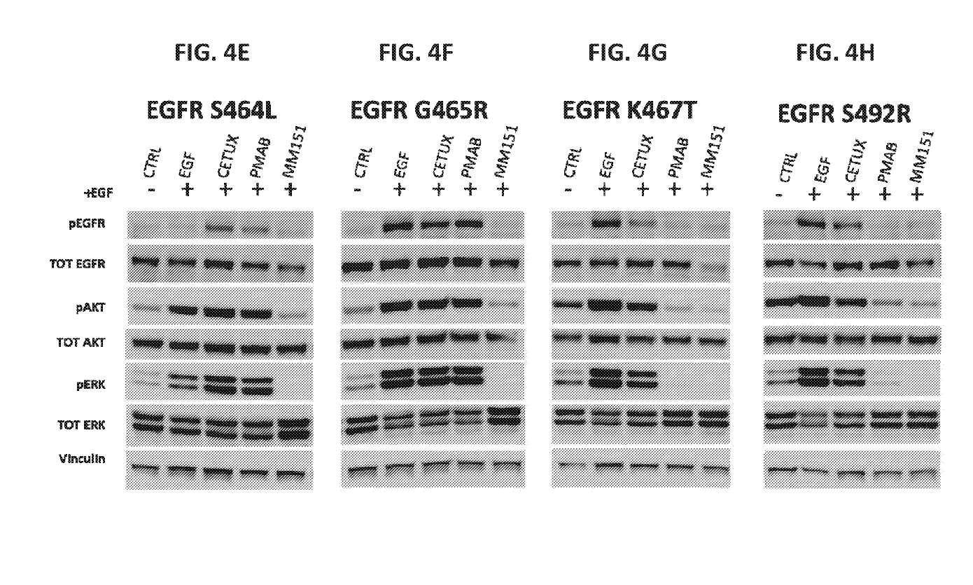

To demonstrate that the growth inhibition observed in the previous Example is due to blockage of the EGF signaling cascade, LIM1215 human colorectal carcinoma cells were infected with lentivirus expressing empty vector control (FIG. 4A), wild-type EGFR (FIG. 4B), or the EGFR extracellular domain mutants including R451C, (FIG. 4C), S464L (FIG. 4D), K467T (FIG. 4E), G465R (FIG. 4F), I491M (FIG. 4G), and S492R (FIG. 4H). Selected cells were cultured in the presence of cetuximab (“CETUX”), panitumumab (“PMAB”) or MM-151 for 2 hours and stimulated with EGF (5 ng/ml) for 15 minutes. Immunoblotting was performed using antibodies to phospho-EGFR, (“pEGFR”), total EGFR (“TOT EGFR”), phospho-AKT (“pAKT”), total AKT (“TOT AKT”), phospho-ERK (“pERK”), total ERK (“TOT ERK”), and vinculin as a loading control.

As shown in FIGS. 4A-4H, MM-151 is able to inhibit EGFR downstream signaling in all control cells and cells expressing EGFR extracellular domain mutants. In contrast, cetuximab was not effective at inhibiting signaling in cells expressing the K467T or S492R mutations, and neither cetuximab nor panitumumab were effective at suppressing signaling in cells expressing the S464L, G465R, or I491M mutations.

### Example 5

### MM-151 Effectively Inhibits Proliferation of LIM1215 Cetuximab-Resistant Clonal Cell Populations when EGFR Ectodomain Mutations are Expressed

LIM1215 wild-type cells, LIM1215 resistant cell populations (i.e., cell populations with a mixture of wild-type and resistant cells, some of which harbor an EGFR-ECD mutation), and LIM1215 resistant clones (i.e., an isolated cell expressing an EGFR-ECD mutation that was grown into a clonal population) were treated for six days with increasing concentrations (0.01 μg/ml-100 μg/ml) of cetuximab, panitumumab or MM-151. Cell viability was measured by the adenosine triphosphate (ATP) assay described above. All assays were performed independently at least three times. CMAB: cetuximab; PMAB: panitumumab.

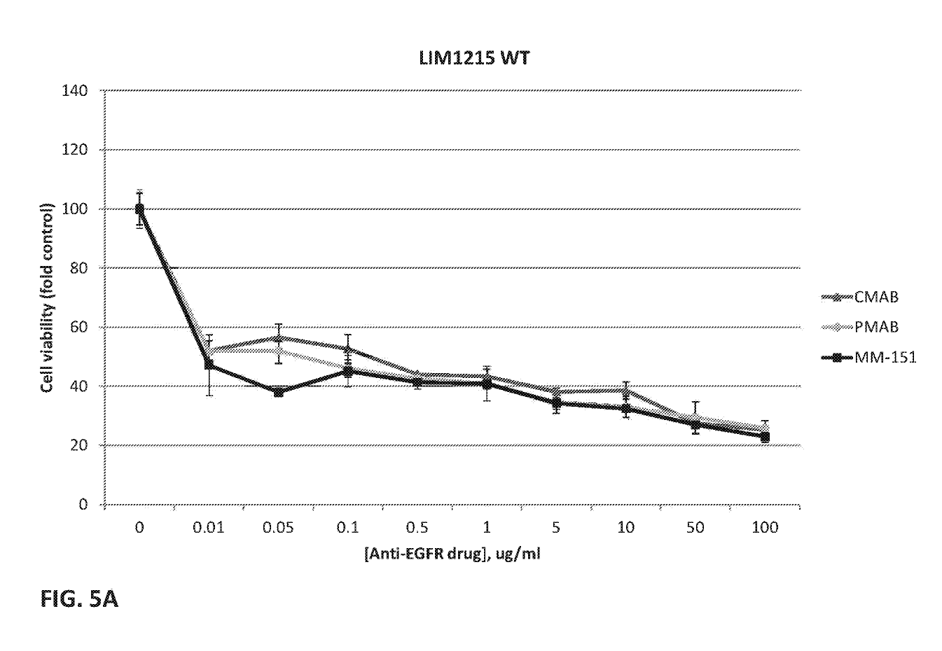

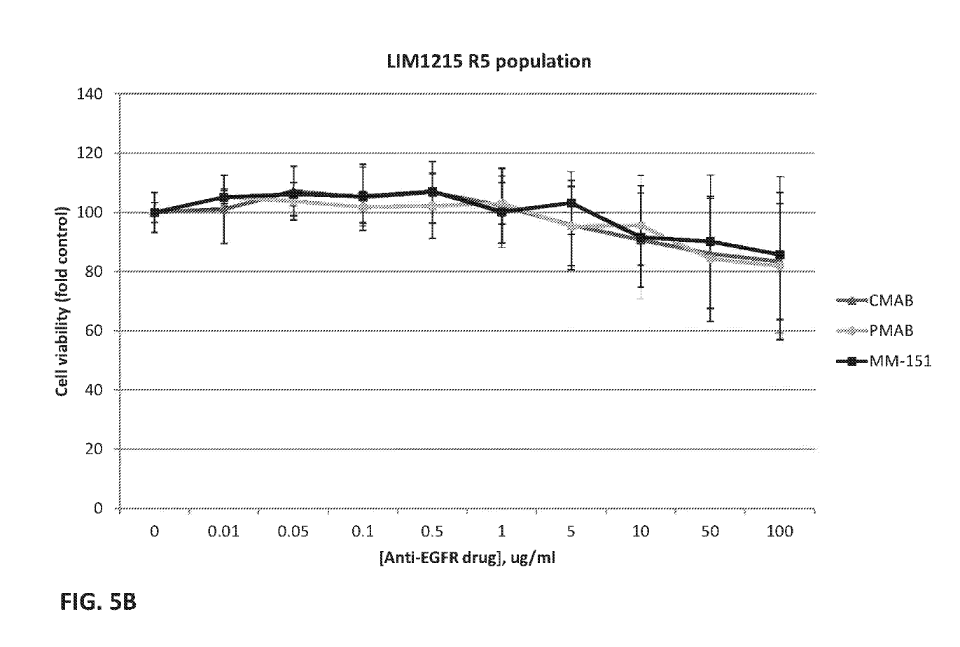

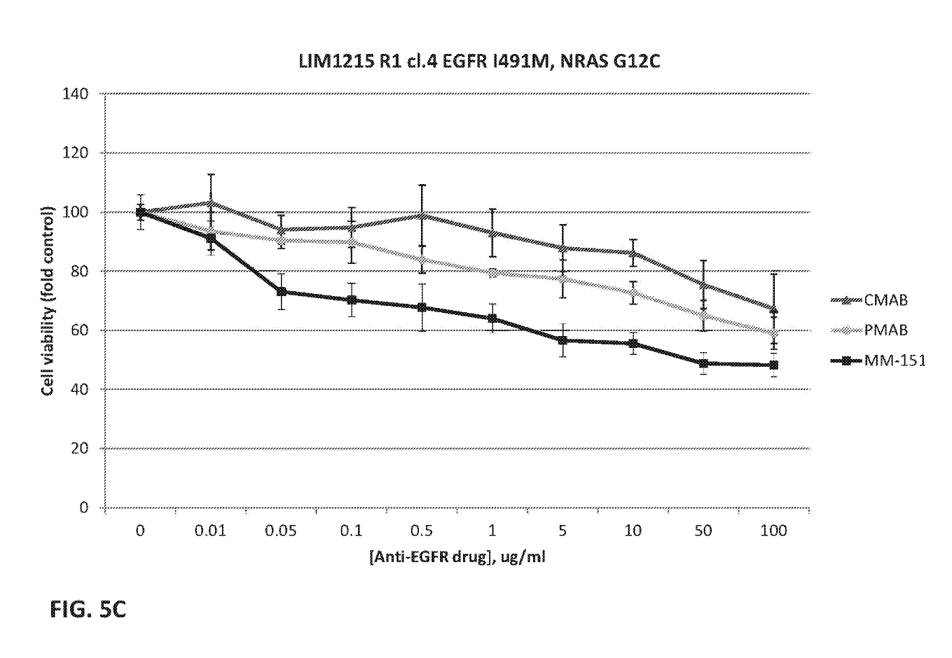

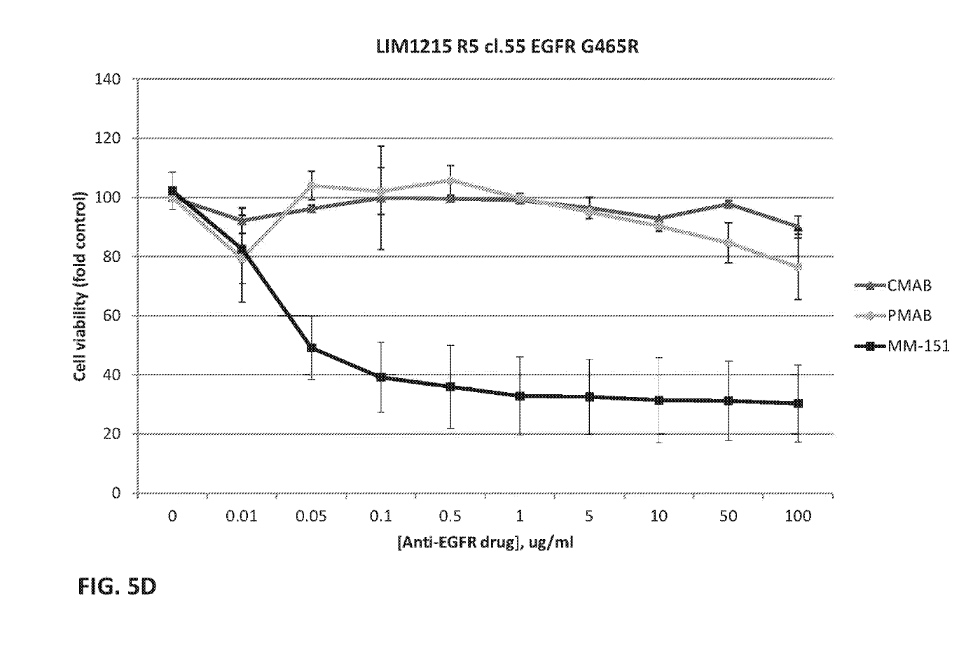

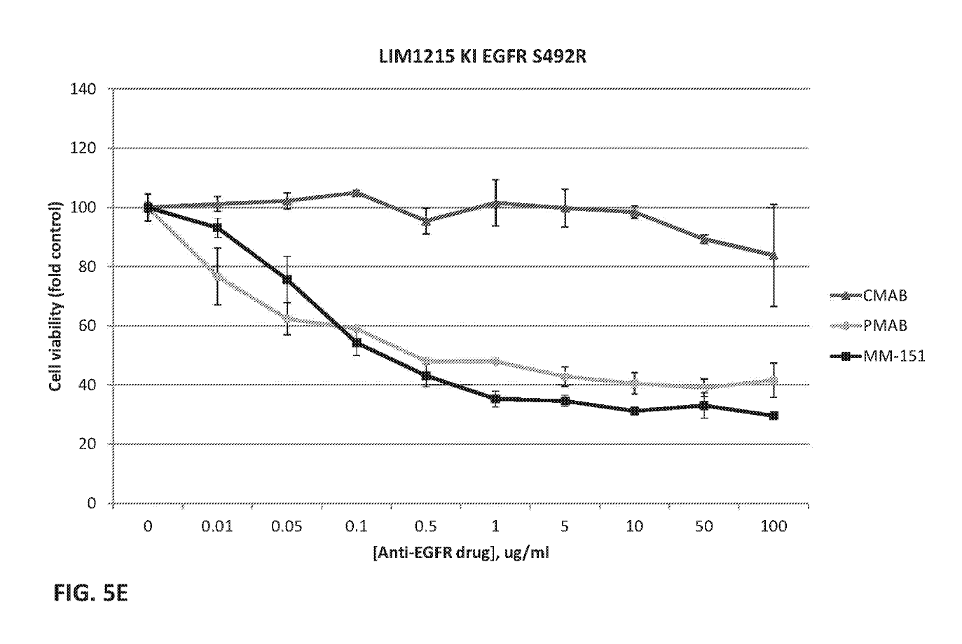

As shown in FIG. 5A, all three drugs were able to inhibit cell viability in wild-type cells. In contrast, none of the three drugs were able to strongly inhibit the cell viability of one resistant population of cells (FIG. 5B, “LIM1215 R5 pop”). Only MM-151 was able to inhibit the cell viability of a second resistant population of cells (FIG. 5C, “LIM1215 R5 pop EGFR G465R”) wherein the population of cells carries the EGFR-ECD mutation G465R. In a clonal population harboring the EGFR-ECD mutation I491M (FIG. 5D), none of the three drugs was able to inhibit cell viability to a great degree, although there was a slight decrease in viability of the cells treated with MM-151. In a clonal population harboring the EGFR-ECD mutation S492R (FIG. 5E), only cetuximab failed to inhibit cell growth. Together, these results confirm the observations seen in the above examples for the effectiveness of each of the three drugs against cells expressing EGFR-ECD mutants.

### Example 6

### MM-151 Mildly Affects Proliferation of OXCO-2 Cetuximab-Resistant Cells

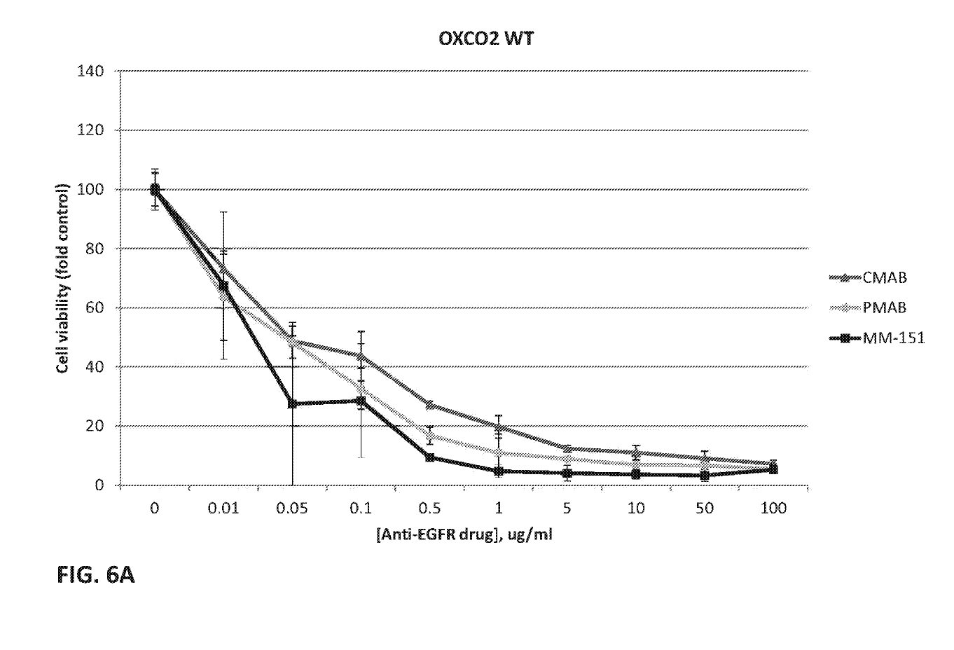

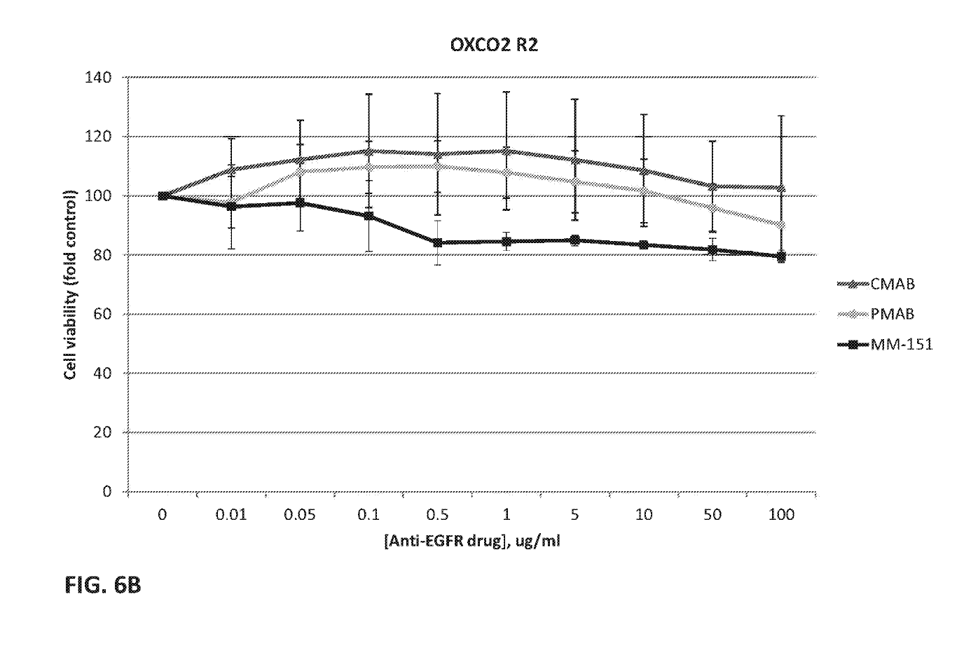

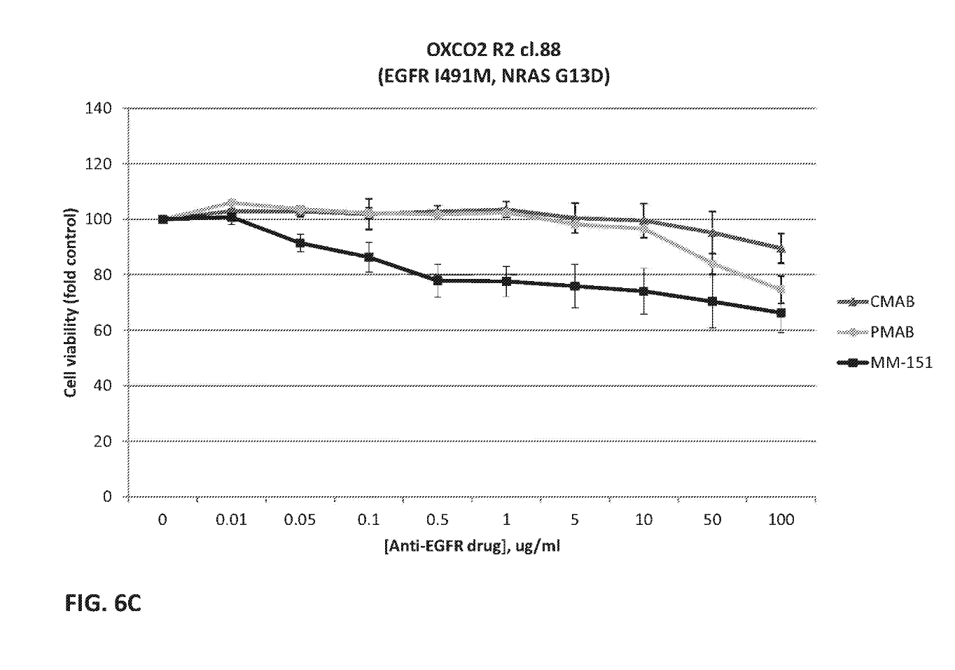

As with LIM1215 wild-type cells, OXCO-2 cells endogenously express EGFR and are sensitive to anti-EGFR therapeutics. Therefore, OXCO-2 cells were evaluated using the methods described above in Example 5. As shown in FIG. 6A, all three drugs were able to inhibit cell viability in wild-type cells. In contrast, none of the three drugs were able to strongly inhibit the cell viability of one resistant population of cells (FIG. 6B) although MM-151 appeared to have a slight effect on cell viability compared to cetuximab and panitumumab; a similar result was seen in FIG. 6C, wherein the cells are a clonal population expressing the EGFR ECD mutation I491M.

### Example 7

### MM-151 Effectively Inhibits Proliferation of CCK81 and HCA46 Cetuximab-Resistant Cells Expressing High Level of EGFR Ectodomain Mutations

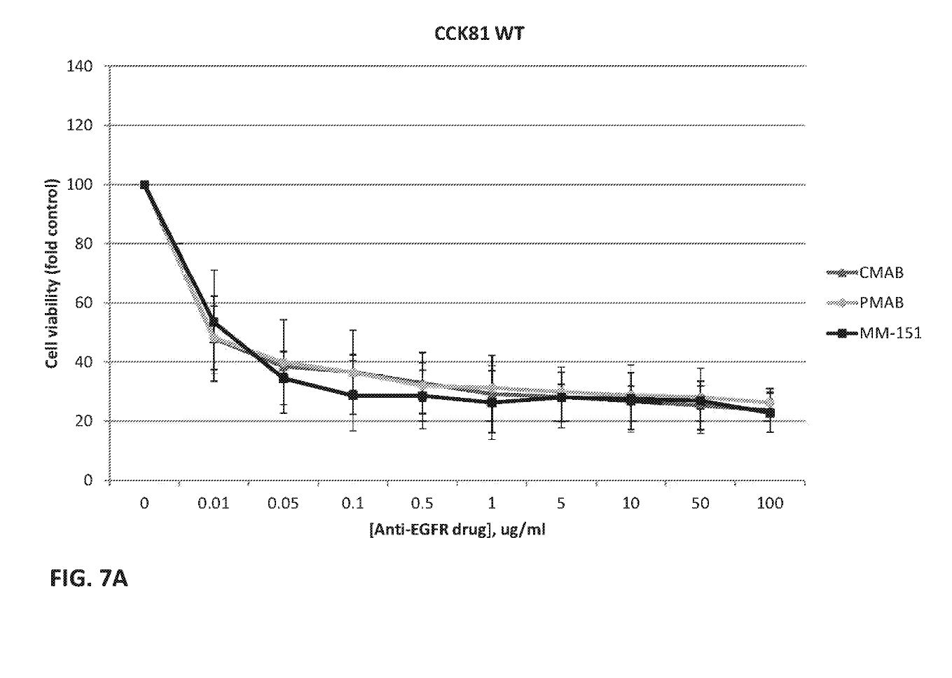

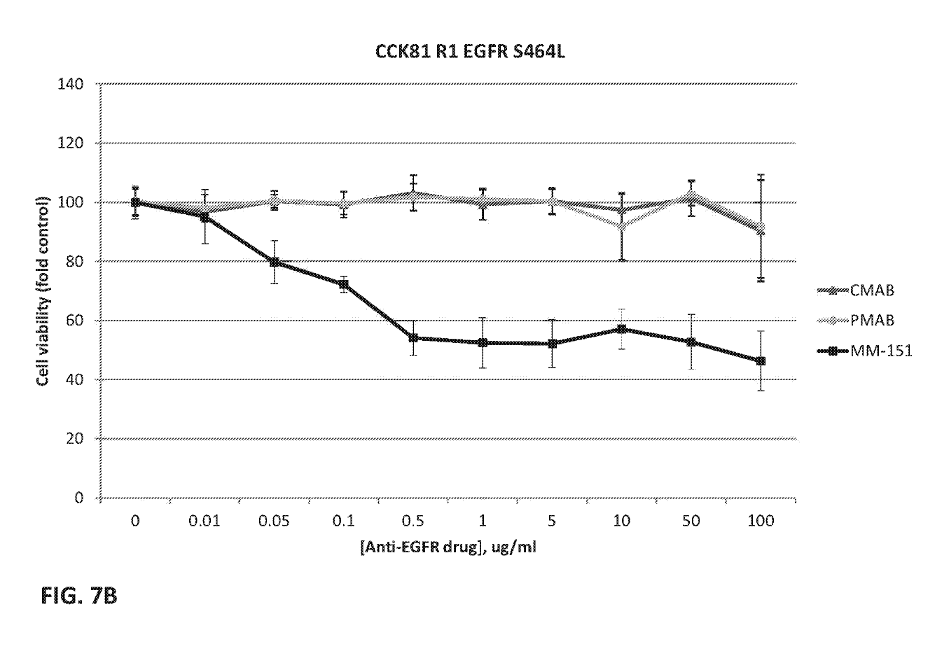

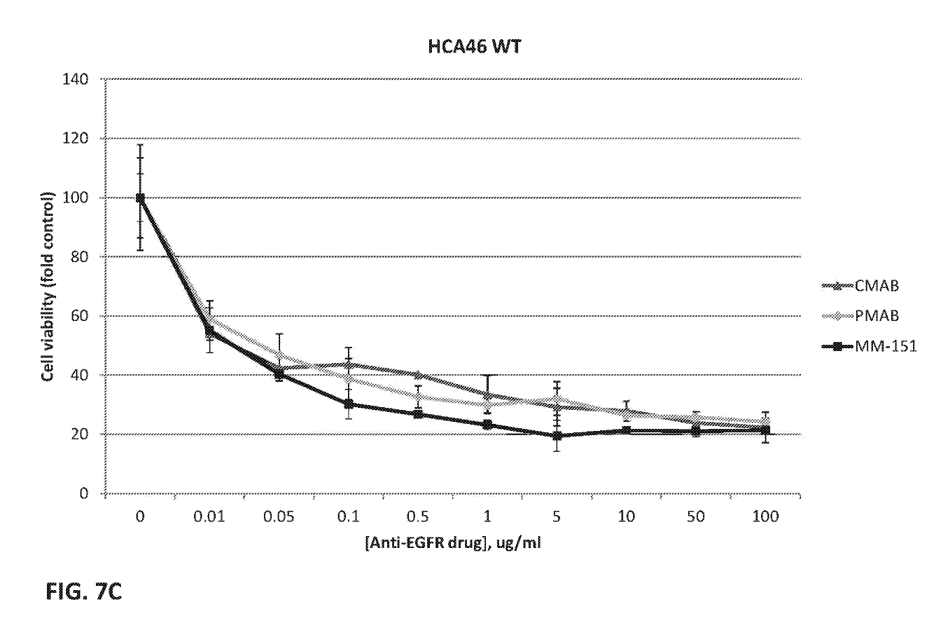

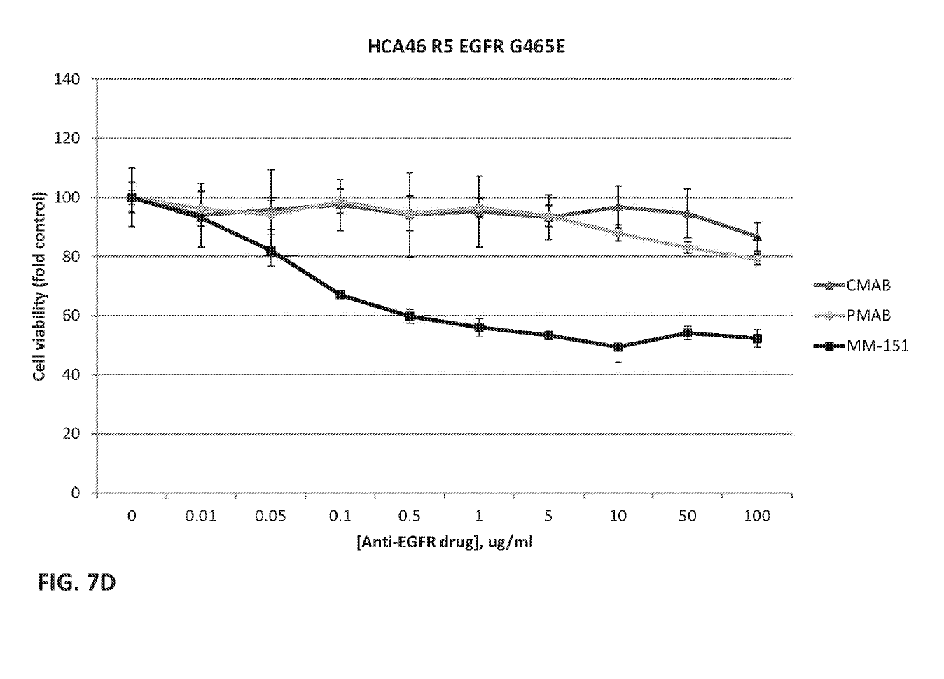

As described for the cells in Example 5, CCK81 and HCA46 wild-type and resistant cell populations were treated for six days with increasing concentrations of cetuximab, panitumumab or MM-151. Cell viability was measured by the adenosine triphosphate (ATP) assay described in the methods above. As with the other cell lines, all three drugs inhibited the viability of wild-type CCK81 cells (FIG. 7A) and wild-type HCA46 cells (FIG. 7C). In contrast, a CCK81 clonal cell population harboring the S464L mutation was not affected by treatment with cetuximab or panitumumab, while MM-151 was able to inhibit cell viability (FIG. 7B). A similar result was seen for a clonal population of HCA46 cells harboring the G465E mutation (FIG. 7D).

### Example 8

### MM-151 Blocks EGFR Downstream Signaling in Cetuximab Resistant Cells

Wild-type and cetuximab resistant populations and clones were cultured in the presence of cetuximab (“CETUX”), panitumumab (“PMAB”) or MM-151 for 2 hours and stimulated with EGF (10 ng/ml) for 15 minutes. Immunoblotting was performed using antibodies to phospho-EGFR, (“pEGFR”), total EGFR (“TOT EGFR”), phospho-AKT (“pAKT”), total AKT (“TOT AKT”), phospho-ERK (“pERK”), total ERK (“TOT ERK”), and vinculin as a loading control.

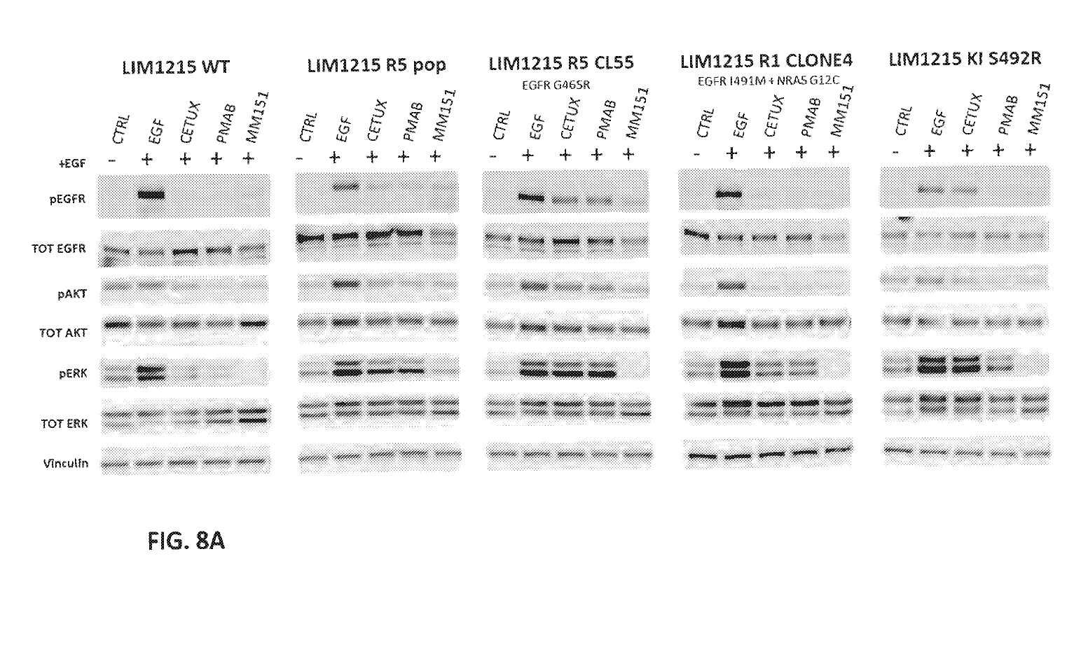

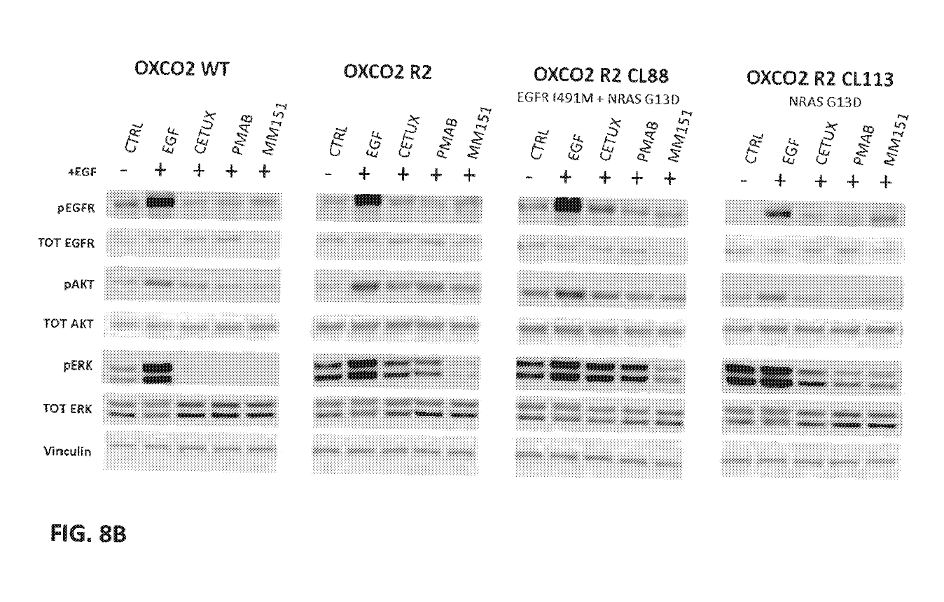

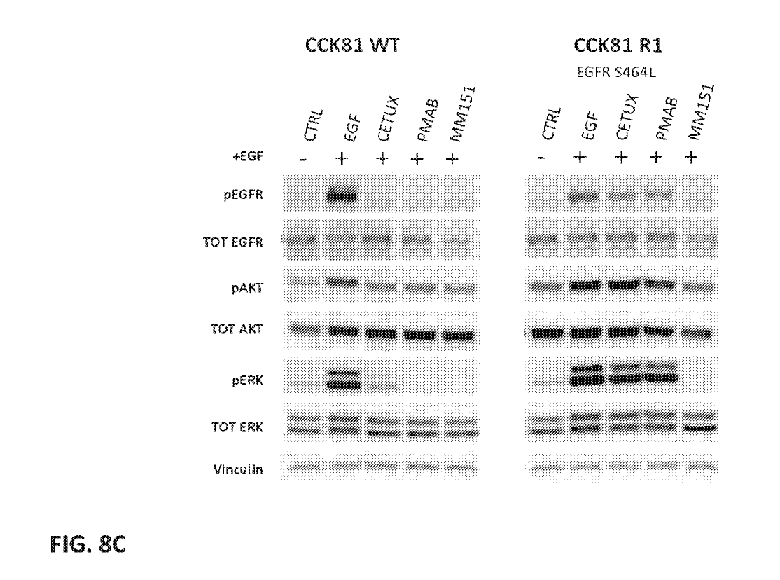

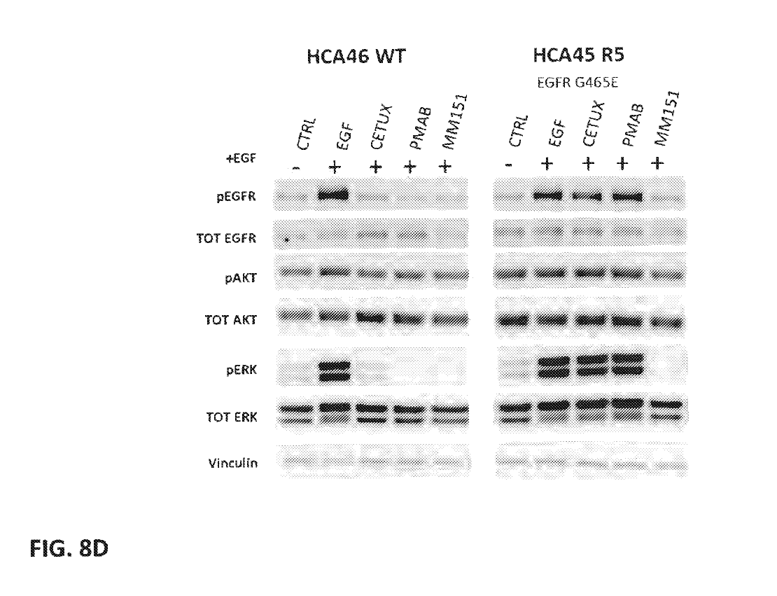

The anti-EGFR-sensitive cell lines LIM1215, OXCO-2, CCK81, and HCA46 were evaluated. As shown in FIGS. 8A-8D, MM-151 was able to inhibit EGFR downstream signaling in all control cells and cells expressing EGFR extracellular domain mutants. In LIM1215 cells, neither panitumumab nor cetuximab were able to inhibit a mixed population of cetuximab-resistant cells (“R5 pop”), a clonal population of cells expressing the EGFR-ECD mutations G465R (“R5 CL55”), I491M (“R1 CLONE4”), or S492R (“KI”) (FIG. 8A). In OXCO-2 cells, neither panitumumab nor cetuximab were able to inhibit a mixed population of cetuximab-resistant cells (“R2”) or a clonal population of cells expressing the EGFR-ECD mutation I491M (“R2 CL88”). In contrast, they slightly inhibited EGFR signaling in a clonal population of cells harboring an NRAS mutation but no EGFR-ECD mutation (“R2 CL113”) (FIG. 8B). In CCK81 cells, all three drugs inhibited the wild-type cells, but only MM-151 inhibited a clonal population of cells harboring the S464L mutation (“R1”) (FIG. 8C). Finally, in HCA46 cells, as seen with the CCK81 cells, all three drugs inhibited the wild-type cells, but only MM-151 inhibited a clonal population of cells harboring the G465E mutation (“R5”) (FIG. 8D).

### Example 9

### MM-151 Effectively Inhibits Proliferation in Xenopatient-Derived Cells and Clones Expressing EGFR Ectodomain Mutation

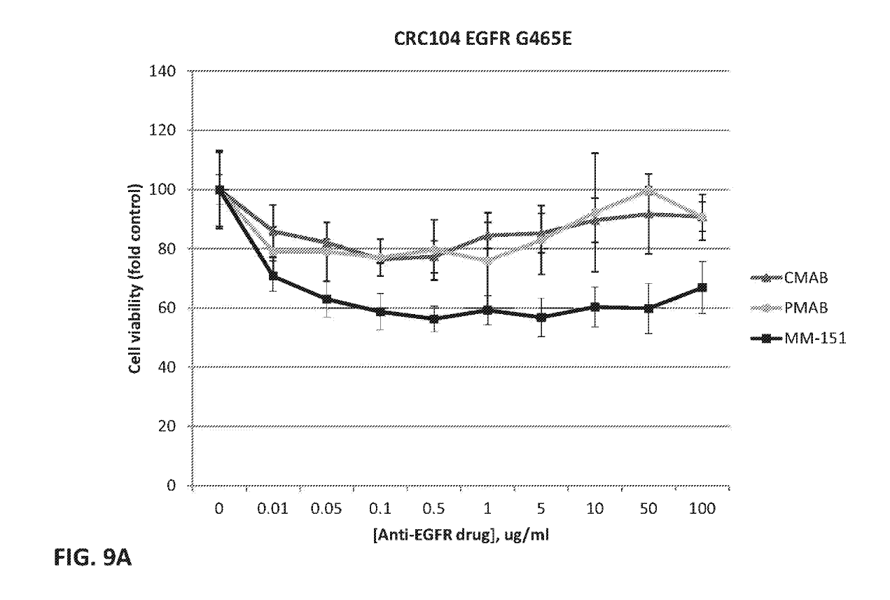

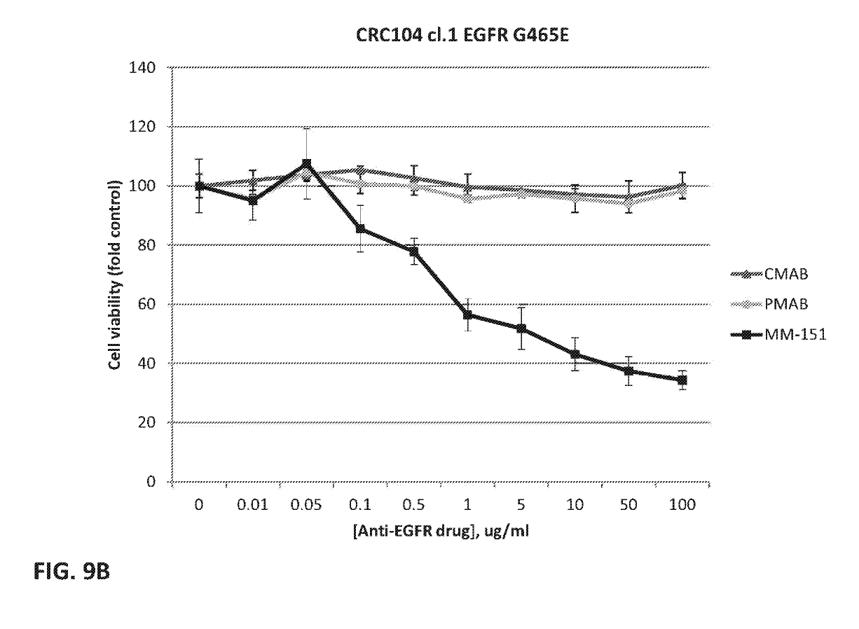

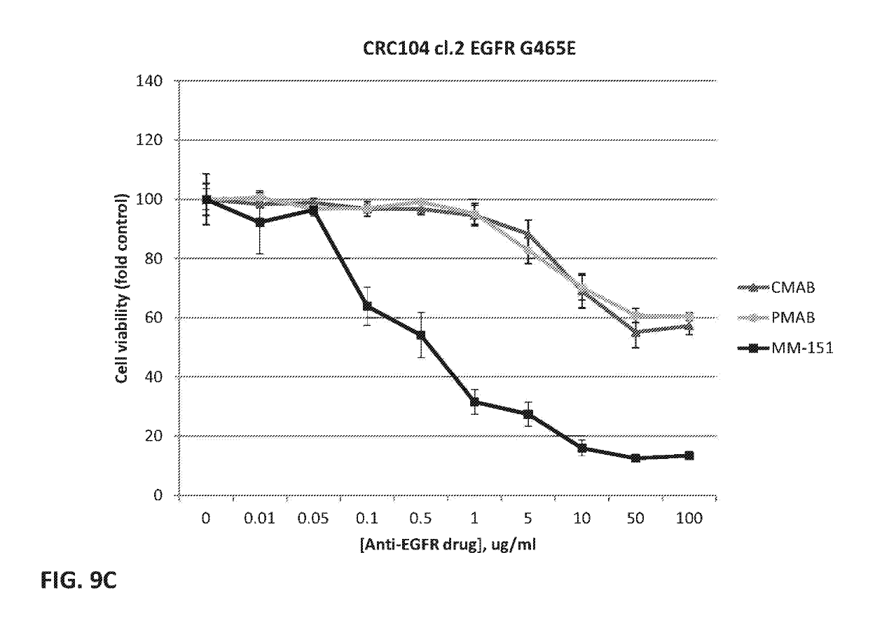

Cells derived from xenopatient iCRC0104 (cells isolated from a patient tumor and then cultured in vitro) (carrying EGFR G465E) and resistant derivative clones were treated for six days with increasing concentrations of cetuximab, panitumumab and MM-151. Cell viability of the CRC 104 culture (FIG. 9A) and two clonal cell populations (cl.1, FIG. 9B, and cl.2, FIG. 9C) was measured by the adenosine triphosphate (ATP) assay described above. In all three cultures, MM-151 was able to inhibit cell viability, whereas cetuximab and panitumumab were only able to inhibit viability very slightly in the mixed cell population (FIG. 9A), and only at very high concentrations in the second clonal cell population (FIG. 9C). Cetuximab and panitumumab did not inhibit cell viability of the first clonal cell population (FIG. 9B).

### Example 10

### Binding of Three Antibody Mixtures to EGFR-ECD Mutants

Unlike monoclonal antibody preparations that exclusively bind Domain III of the EGFR-ECD domain, the MM-151 antibody mixture, which binds other parts of the extracellular domain in addition to Domain III, is effective at binding to many of the EGFR-ECD mutants. In order to determine whether other mixtures will result in the same activity, a cell-free binding experiment was performed as described below.

All reagents were equilibrated and samples brought to room temperature prior to the bio-layer interferometry assay (ForteBio®). Prior to loading, streptavidin biosensors were hydrated for 10 min in protein-free blocking buffer (Pierce) to minimize any non-specific binding. Assays were run using the Octet® software and procedure per manufacturer's instruction. The assay consisted of the following steps: 5 min of equilibration in 1×PBS, 1 min of biotinylated antibody loading (10 μg/mL), 1 min of baseline stabilization, 3 min of incubation with the extracellular domain variant of EGFR listed (500 nM), 1 min of baseline stabilization, 3 minute incubations with first listed antibody (P3X or “sym992” at 500 nM), another 1 min of baseline stabilization followed by 3 minute incubation with second listed antibody (P1X or 25E at 500 nM). 1×PBS was used as the matrix throughout. Data was analyzed and processed with Octet® Data Analysis software.

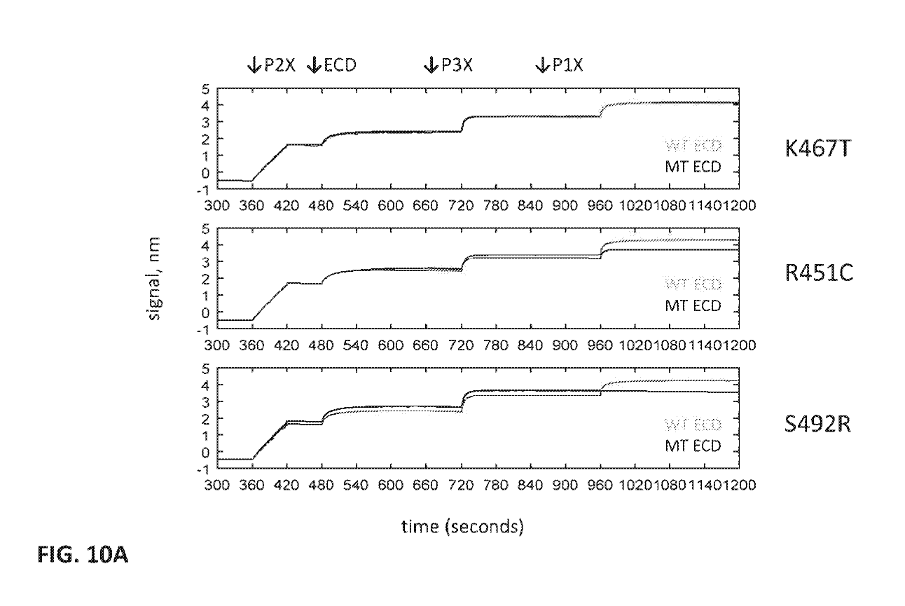

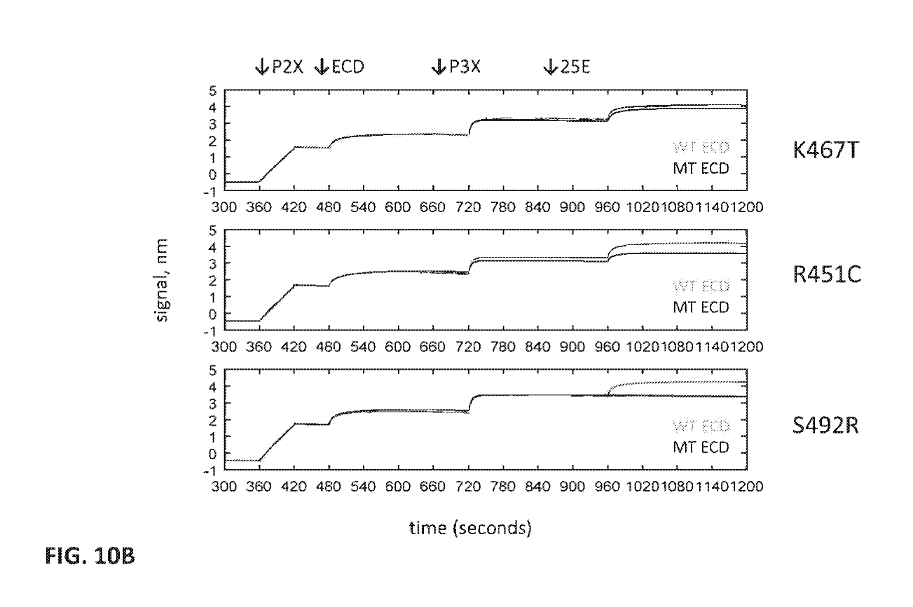

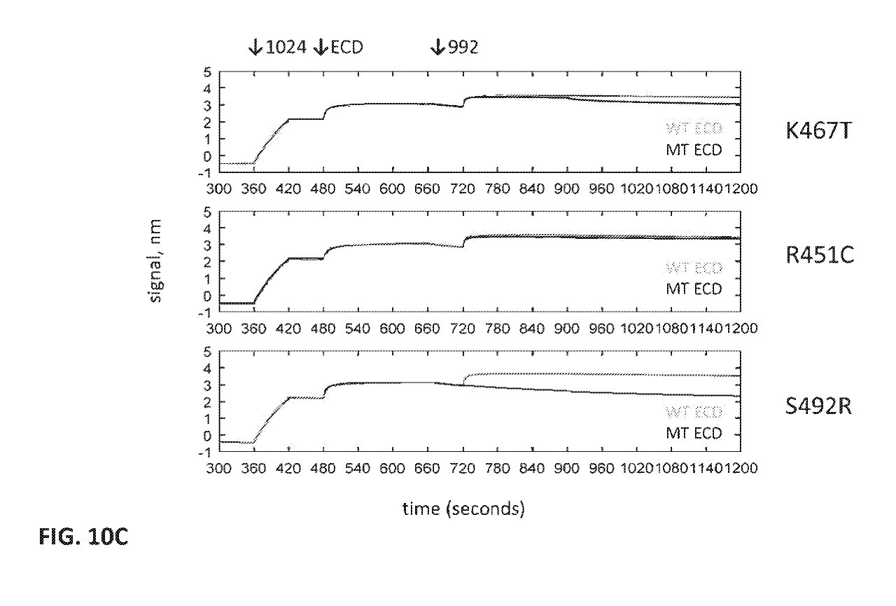

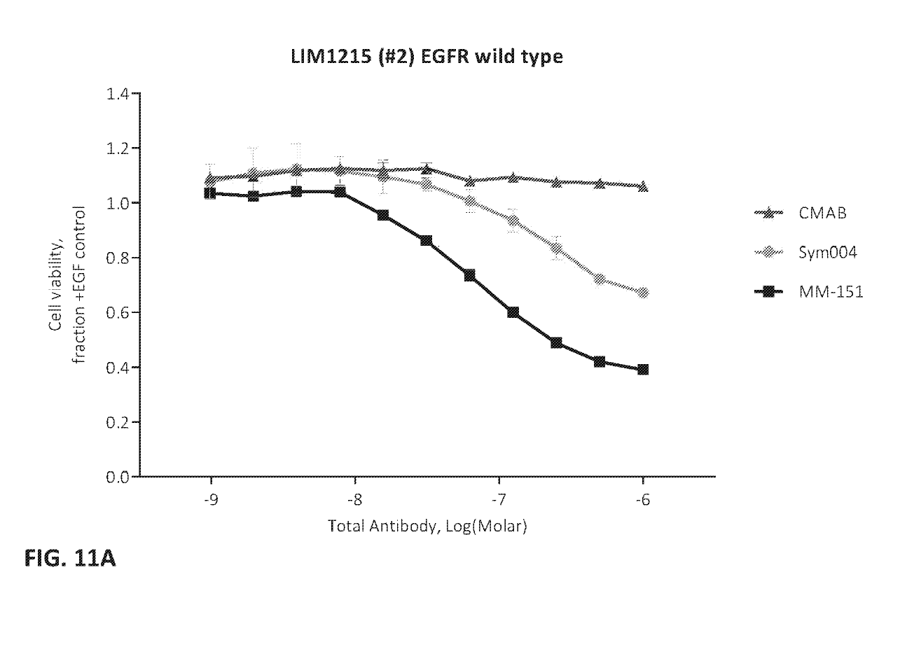

FIG. 10A shows the cell-free binding experiment evaluating MM-151. Three ECD mutants, K467T, R451C, and S492R, were successfully bound to the antibody P2X, and for all three ECD mutants P3X was able to also bind. P1X, which binds to Domain III of the ECD, was able to bind the K467T and R451C ECD mutants, but not the S492R mutant. Similar results were seen in FIG. 10B, wherein P1X was replaced by a similar antibody 25E. A two-antibody mixture, shown in FIG. 10C, while able to bind all three mutant extracellular domains with a first antibody (“sym1024”), was able to bind the K467T and R451C ECD mutants, but not the S492R mutant. Together, these results confirm the observations seen in the above examples for the ability of the MM-151 oligoclonal mixture to engage EGFR ectodomain mutants with at least two antibodies. Further, these results demonstrate that the effectiveness is observed for MM-151 and the related combination of three antibodies, but not for the oligoclonal mixture of sym-992 and sym-1024.

### Example 11

### LIM1215 Overexpressing the EGFR S492R Ectodomain Mutant are Resistant to Both Cetuximab and Sym004 and are Sensitive to MM-151

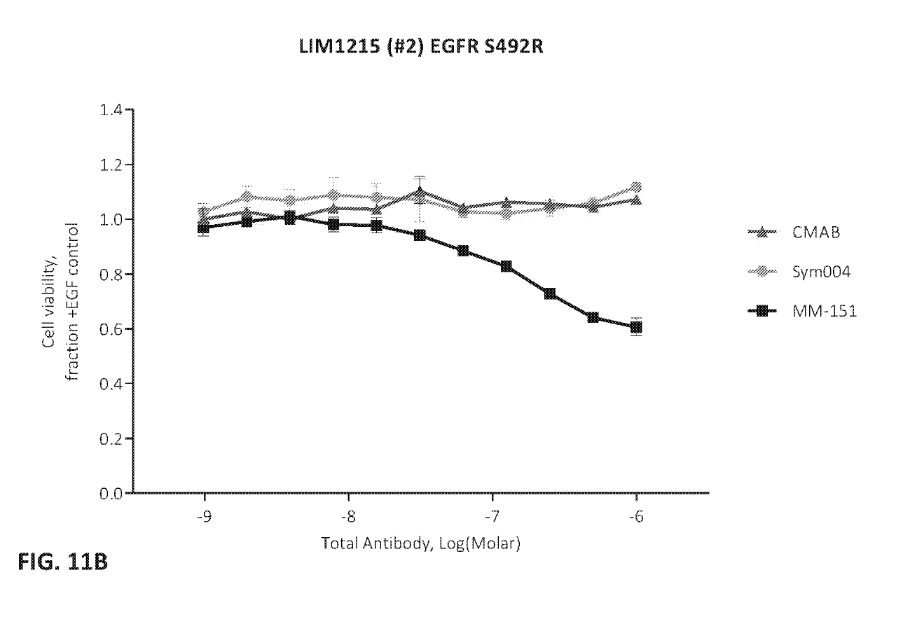

The LIM1215 (#2) cell line was stably transfected with a plasmid expressing the EGFR S492R ectodomain mutation. Selected cells were treated for 3 days with increasing concentrations of cetuximab (“CMAB”, dark grey line), the two-antibody mixture of sym-992 and sym-1024 (“Sym004”, light grey line) and MM-151 (black line) in the presence of 8 nM EGF ligand. Cell viability was measured by the adenosine triphosphate (ATP) assay. As shown in FIGS. 11A and 11B, cetuximab was not able to inhibit growth of cells expressing wild type EGFR (untransfected control) or the EGFR S492R mutation and the Sym004 two-antibody mixture was not able to inhibit growth of cells expressing the EGFR S492R mutation. By contrast, MM-151 was able to inhibit cell growth of cells expressing wild type EGFR and the EGFR S492R mutation.

### Example 12

### LIM1215 Expressing or Overexpressing the EGFR S492R Ectodomain Mutant are Resistant to Both Cetuximab and are Sensitive to MM-151 in an In Vivo Xenograft Model

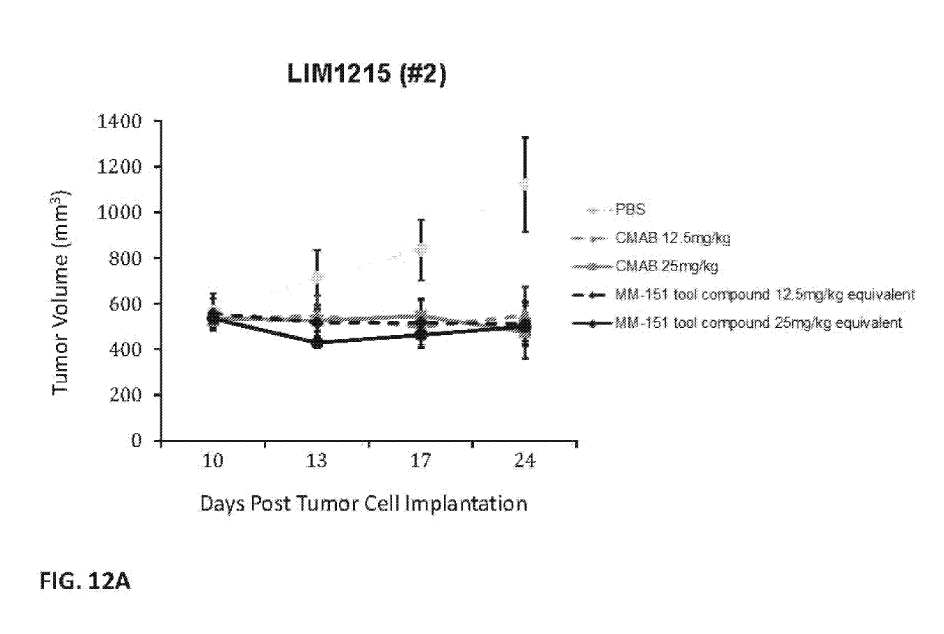

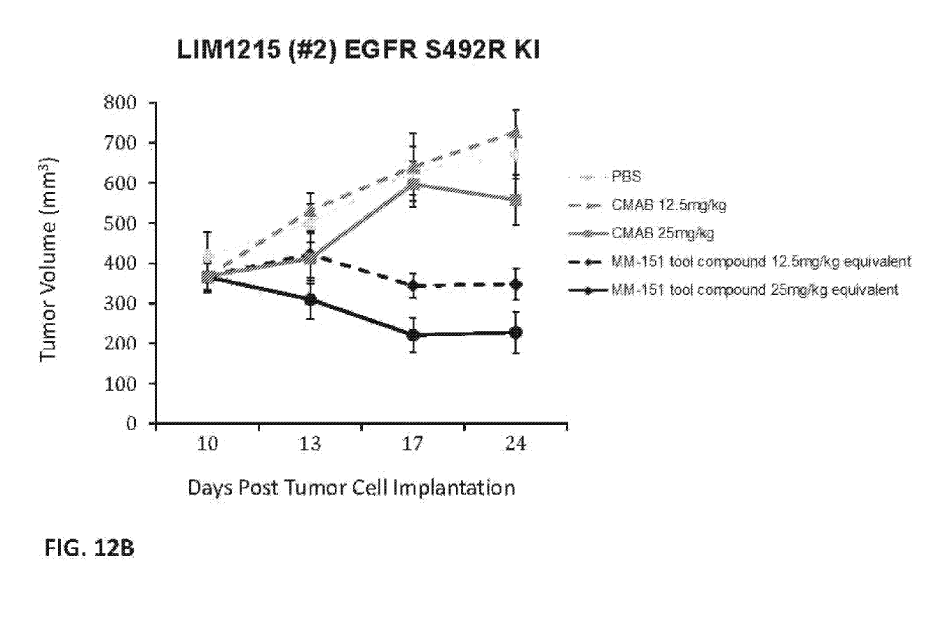

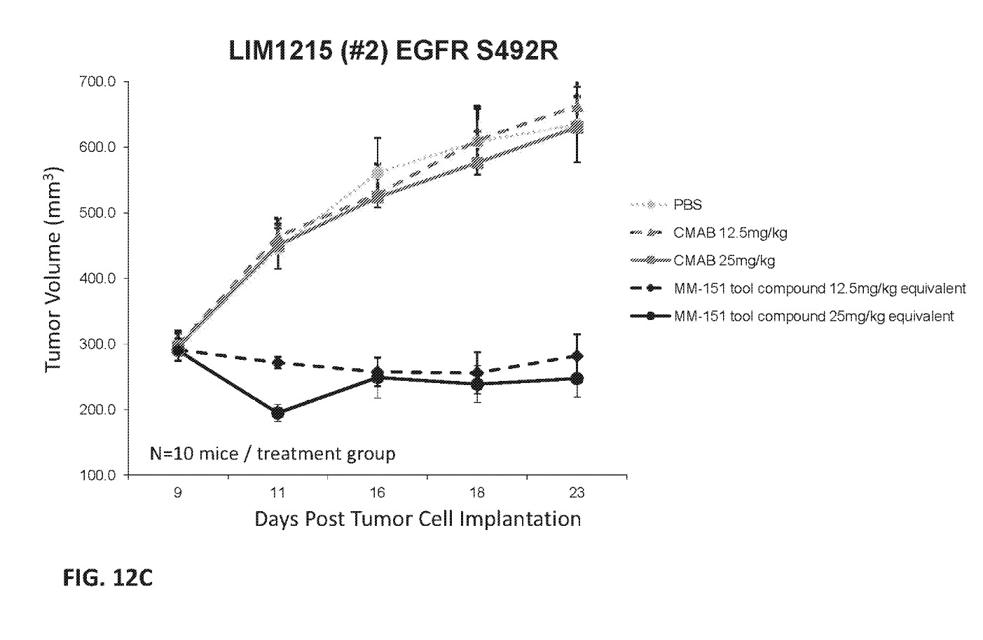

A murine xenograft efficacy study was performed in female nu/nu mice inoculated with the following three cell lines—LIM1215 (#2), LIM1215 (#2) EGFR S492R KI (engineered to express the EGFR S492R ectodomain mutation), or the LIM1215 (#2) EGFR S492R (stably transfected with a plasmid expressing the EGFR S492R ectodomain mutation). Tumors were allowed to grow to approximately 300 mm³ and the mice were then randomized into treatment groups of 10. Following randomization, mice were treated with PBS (vehicle control), cetuximab, or MM-151 tool compound for two weeks. As shown in FIG. 12A, the parental LIM1215 (#2) cell line is sensitive to both cetuximab and MM-151 at the two indicated dose levels. As shown in FIGS. 12B and 12C, cetuximab was not able to inhibit tumor growth of the cell lines harboring expression (FIG. 12B) or over-expression (FIG. 12C) of the EGFR S492R ectodomain mutation at the two indicated dose levels. By contrast, MM-151 was able to inhibit tumor growth at both dose levels.

### Example 13

### Longitudinal Monitoring of EGFR ECD Mutations in Circulating Cell-Free DNA of Patients can Function as Marker of Response to MM-151 Therapy

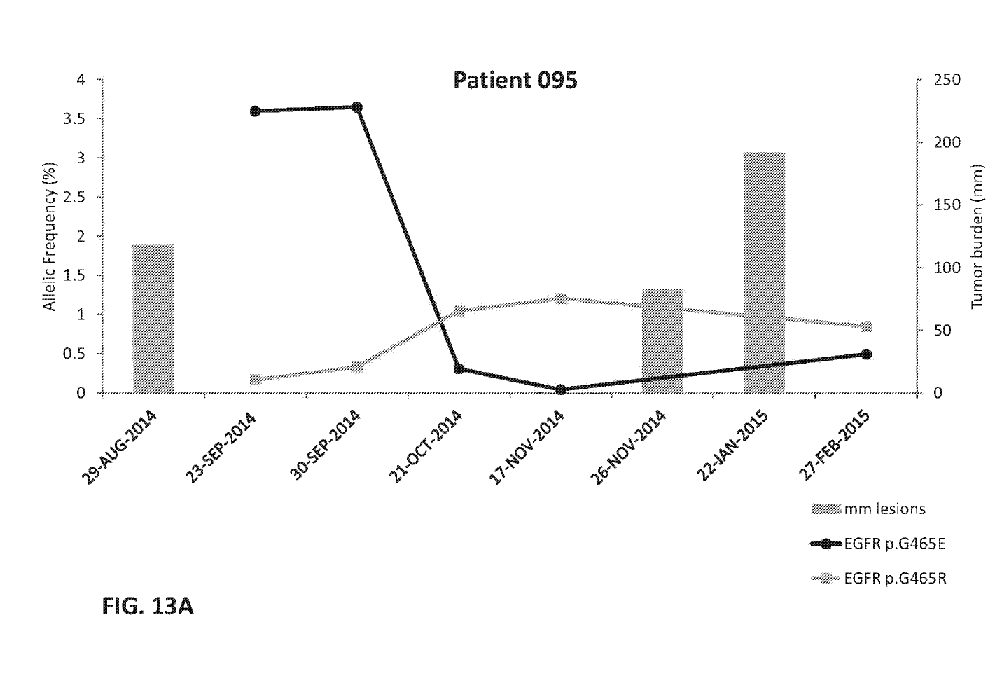

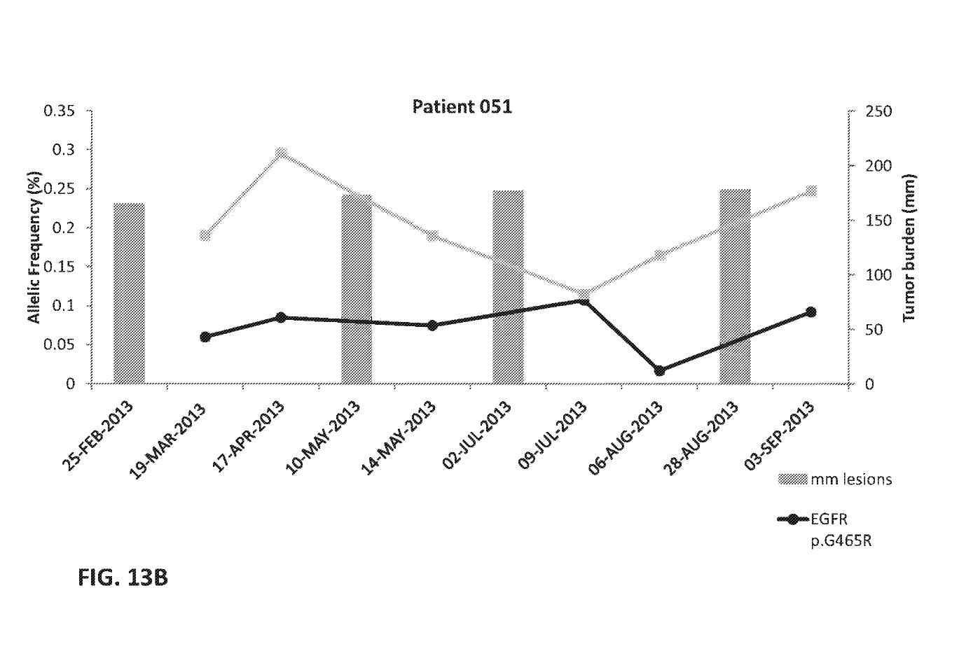

An analysis of EGFR ECD mutations in circulating cell-free DNA was performed on a subset of serum samples collected on a MM-151 phase 1 clinical study (NCT #01520389)(25). The subset included 11 colorectal cancer patients with documented prior exposure to anti-EGFR antibodies (e.g. cetuximab, panitumumab) for whom appropriate serum samples were available. EGFR ECD mutations were detected by droplet digital PCR in the baseline samples preceding the first administration of MM-151 in 2 of 11 patients. Longitudinal analysis performed in samples collected during the course of MM-151 treatment, highlighted that the allelic frequencies of EGFR ECD mutations in cell-free DNA changed during MM-151 administration. The stark reduction in the allelic frequency of the EGFR G465E mutation observed in patient 095 anticipated the significant reduction in tumor volume that was measured approximately four weeks later by CT scan (FIG. 13A). A reduction and stabilization in EGFR S464L and G465R mutations, respectively, accompanies the prolonged disease stabilization observed in patient 051 (FIG. 13B). The reverse of the decline in the allelic frequencies of these mutations anticipated the progression at 24 weeks.

### Example 14

### Binding of Individual Antibodies to Cells that Express the EGFR S492R Mutation

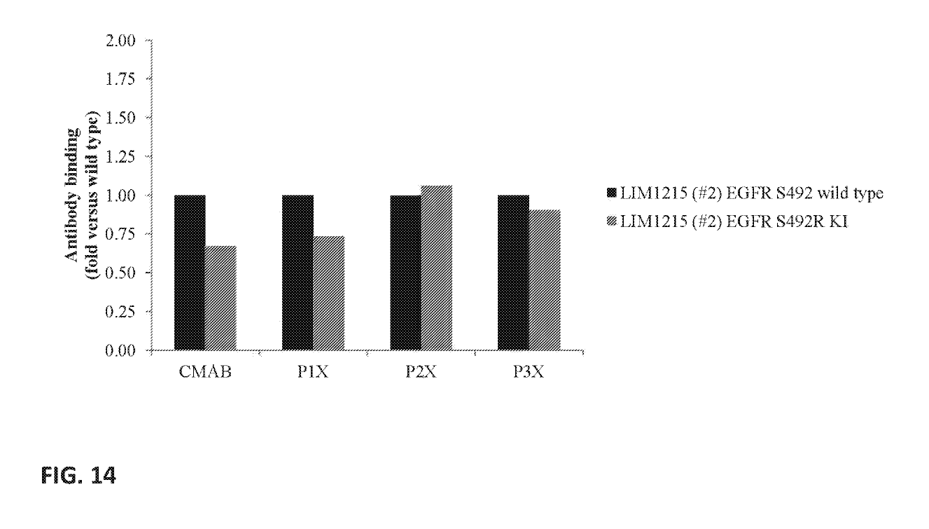

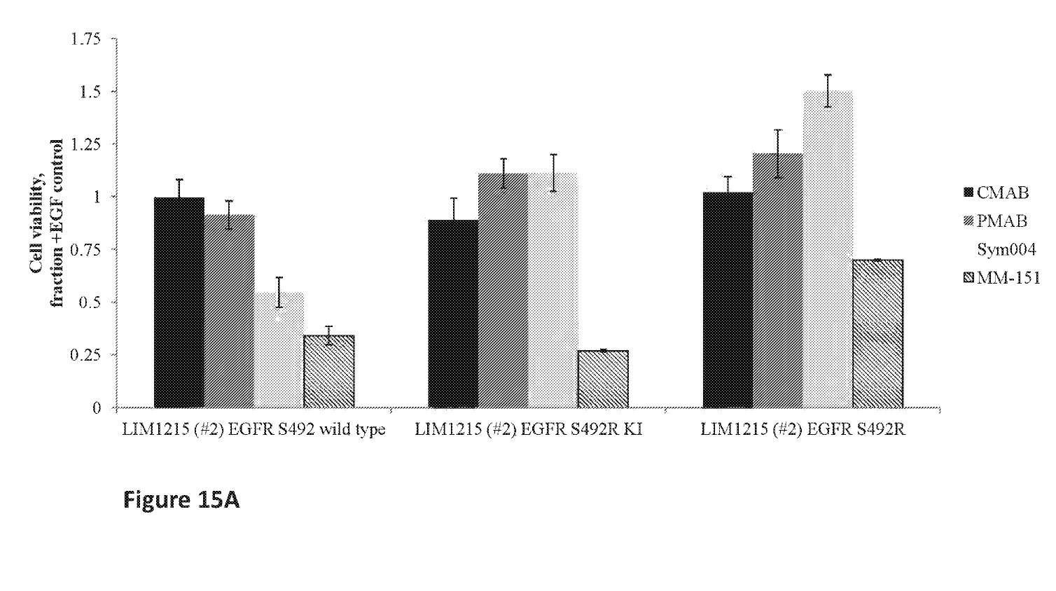

A flow cytometry binding experiment was performed to assess the degrees to which cetuximab (“CMAB”) and the three component antibodies of the MM-151 mixture (P1X, P2X, and P3X) bind to cells engineered to express the EGFR S492R ectodomain mutation (“LIM1215 (#2) EGFR S492R KI”, gray bars)(FIG. 14). Cells were incubated on ice (4° C.) for 1 hour with a saturating concentration (0.1 μM) of Alexa-488-labeled antibody. For each antibody, mean fluorescence intensity (MFI) values were measured and normalized to the MFI with EGFR wild type control (LIM1215 (#2) EGFR S492 wild type, black bars). Consistent with the results from the cell-free binding assay described in Example 10, the binding of cetuximab and P1X is reduced on cells engineered to express the EGFR S492R ectodomain mutation while binding of P2X and P3X antibodies are unaffected.

### Example 15

### LIM1215 Overexpressing or Engineered to Express the EGFR S492R Ectodomain Mutant are Resistant to Cetuximab, Panitumumab, and Sym004, and are Sensitive to MM-151

A cell viability experiment was performed with cells expressing wild type EGFR (LIM1215 (#2) EGFR S492 wild type), overexpressing the EGFR S492R mutation in addition to wild type EGFR (LIMI1215 (#2) EGFR S492R), and expressing the EGFR S492R mutation via gene editing (LIM1215 (#2) EGFR S492R KI). Cells were treated for 3 days with cetuximab (“CMAB”), panitumumab (“PMAB”), the two-antibody mixture of sym-992 and sym-1024 (“Sym004”) and MM-151 in the presence of 8 nM EGF ligand. Cell viability was measured by the adenosine triphosphate (ATP) assay.

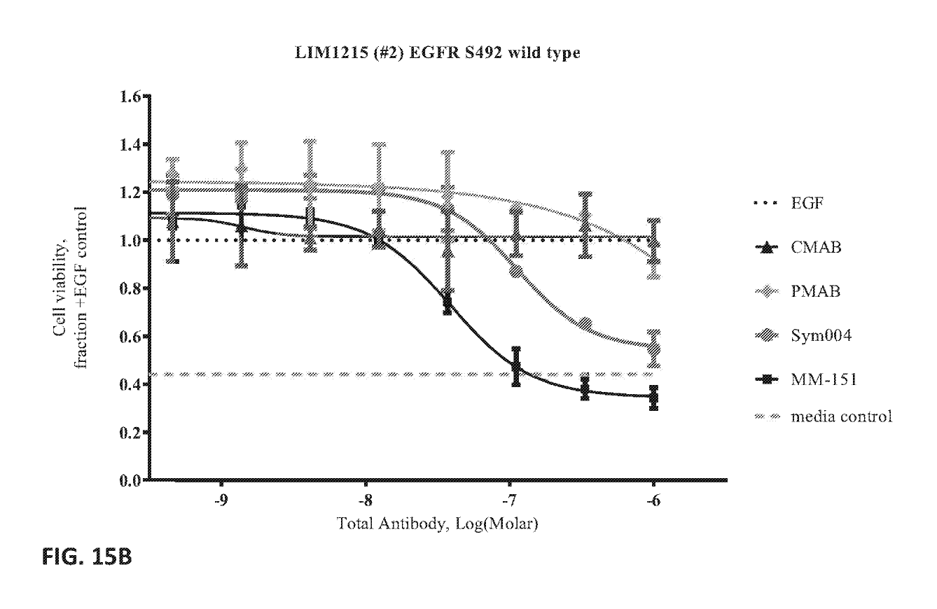

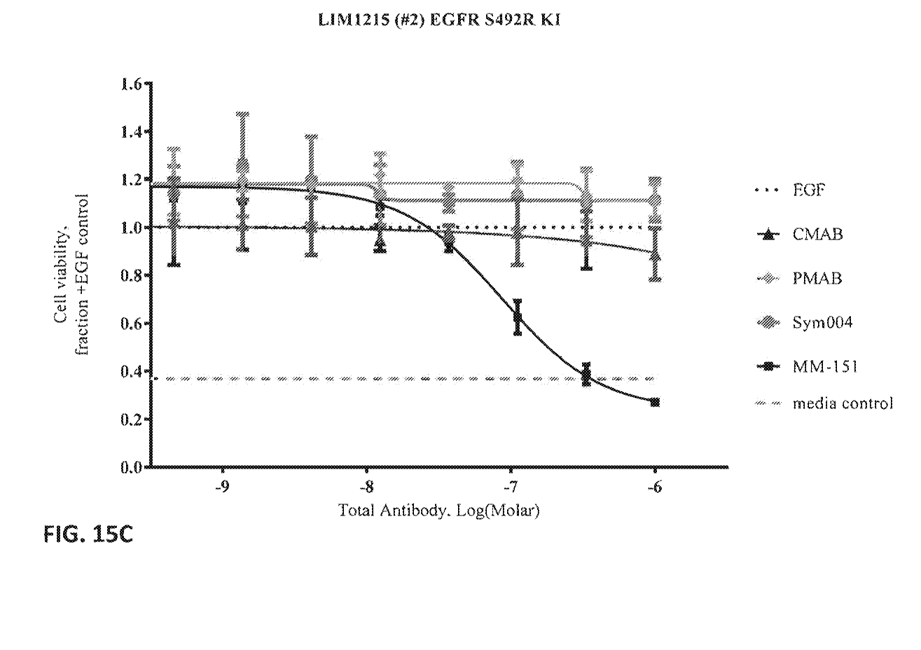

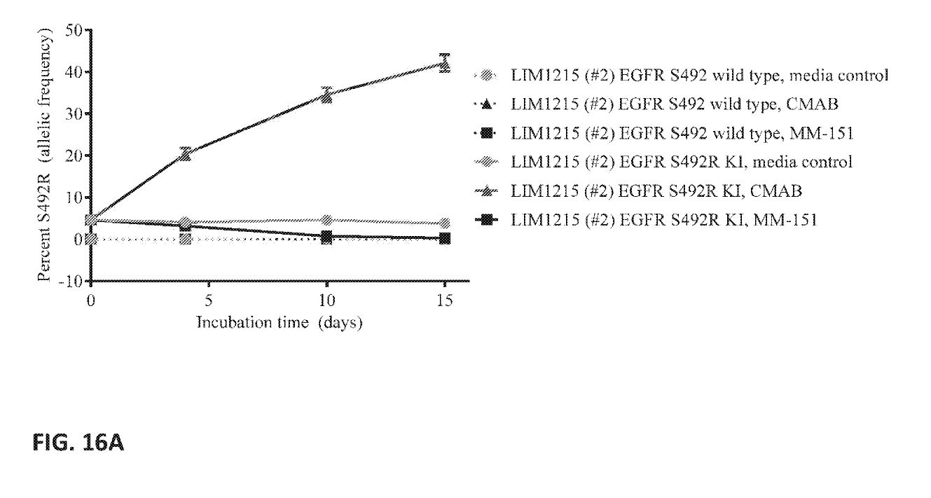

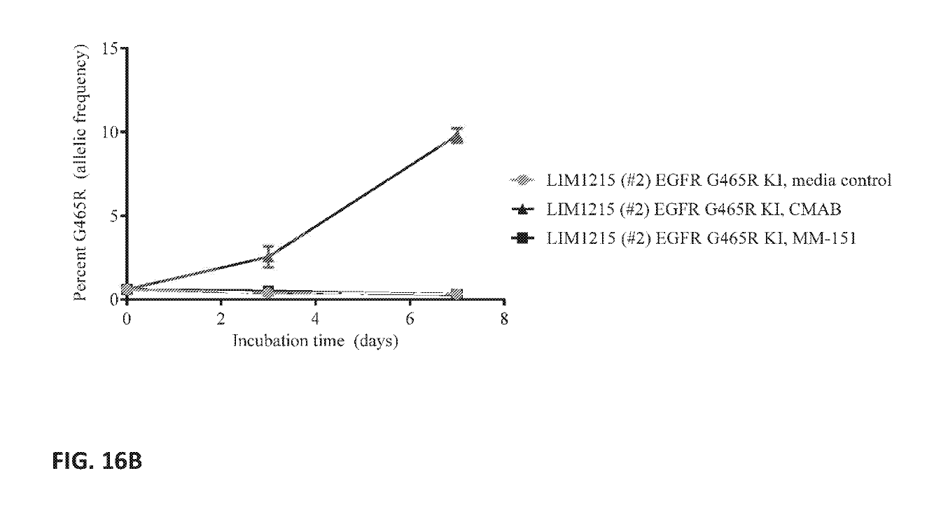

As shown in FIG. 15A, cetuximab (black) and panitumumab (dark gray) were not able to inhibit growth of cells expressing wild type EGFR or the EGFR S492R mutation and the Sym004 two-antibody mixture (light gray) was not able to inhibit growth of cells expressing the EGFR S492R mutation. By contrast, MM-151 (black and white stripe pattern) was able to inhibit cell growth of cells expressing wild type EGFR, overexpressing the EGFR S492R mutation, or engineered to express the EGFR S492R mutation. All antibodies were administered at a concentration of 1 μM.

A second experiment was performed with LIM1215 (#2) EGFR S492 wild type and LIM1215 (#2) EGFR S492R KI cells, but with increasing concentrations of cetuximab (dark gray line with triangle markers), panitumumab (light gray line with diamond markers), Sym004 (gray line with circle markers), or MM-151 (black line with square markers) in the presence of 8 nM EGF ligand (FIGS. 15B and 15C). Cetuximab and panitumumab were unable to inhibit growth of cells expressing wild type EGFR or the EGFR S492R mutation. Sym004 was not able to inhibit cells expressing the EGFR S492R mutation. By contrast, MM-151 was able to inhibit cells expressing wild type EGFR (with an improved potency relative to Sym004) and the EGFR S492R mutation.

### Example 16

### Expansion of Cells Expressing the EGFR S492R or EGFR G465R Mutations is Observed During the Course of Treatment with Cetuximab and is Inhibited by MM-151

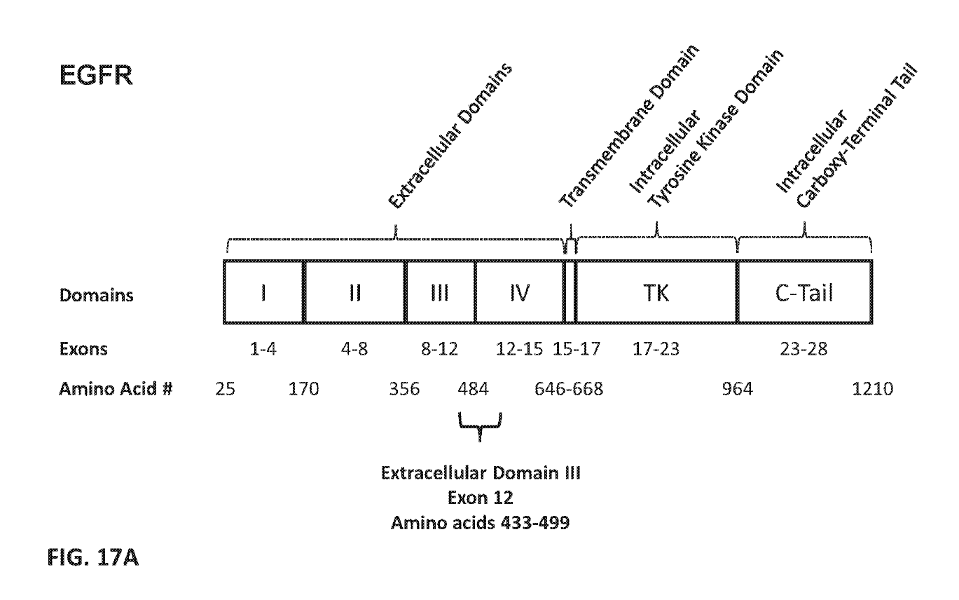

To mimic the selective expansion over time of cells containing an EGFR mutation in a tumor (in which other cells express wild type EGFR), two in vitro pooled culture experiments were performed to assess the change in allelic frequency of two representative EGFR ectodomain mutations—EGFR S492R (FIG. 16A) and EGFR G465R (FIG. 16B)—over the course of treatment with cetuximab (gray line with triangle) markers, MM-151 (black line with square markers), or media alone (light gray line with circle markers). A fifteen or seven day experiment was performed with a pooled culture of LIM1215 (#2) that contains cells engineered to express the EGFR S492R or EGFR G465R ectodomain mutations (solid lines), respectively. As a control in the EGFR S492R experiment, a pooled culture of LIM1215 (#2) cells that express wild type EGFR (engineered with a non-targeting sgRNA to maintain wild type EGFR genotype; stippled lines). Cells were pelleted on Day 0 before treatment (5×10⁵ cells) and on Days 4, 10, and 15 before cells were transferred to a new plate (approximately half the cells in each plate well) and genomic DNA extracted.

The allelic frequency of the EGFR S492R and EGFR G465R ectodomain mutations in the genomic DNA was assessed by droplet digital PCR. As expected, the S492R allelic frequency was measured as 0% in the wild type control wells (regardless of treatment) on all days of the experiment. The mean S492R allelic frequency before treatment (Day 0) in the EGFR S492R wells was measured as 4.6% and remained steady in the presence of media alone over the 15 days (range: 4.6 to 3.8%). Similarly, the mean G465R allelic frequency before treatment (Day 0) in the EGFR G465R wells was measured as 0.6% and remained steady in the presence of media alone over the seven days (range: 0.3 to 0.6%).

The mean allelic frequency in the EGFR S492R wells treated with cetuximab increased from 4.6% to 42.1% over the fifteen day period. In contrast, treatment with MM-151 led to a decrease in the mean S492R allelic frequency in the EGFR S492R wells from 4.6% to 0.3%. Similarly, the mean allelic frequency in the EGFR G465R wells treated with cetuximab increased from 0.6% to 9.8% over the seven day period. In contrast, treatment with MM-151 led to a decrease in the mean G465R allelic frequency in the EGFR G465R wells from 0.6% to 0.3%.

### Example 17

### The Known EGFR Extracellular Domain Mutations are Isolated to a 67 Amino Acid Sequence on EGFR

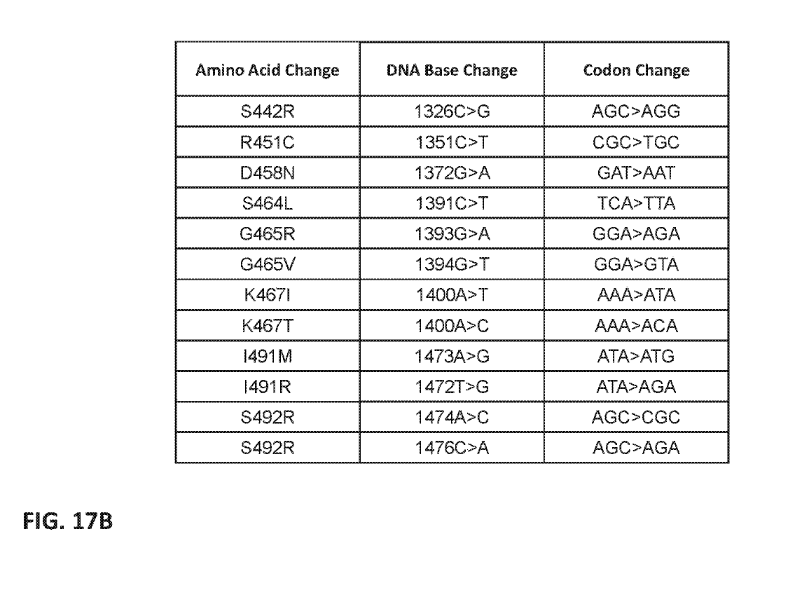

The human EGFR gene is comprised of 28 exons that encode a 1210 amino acid sequence (FIG. 17A). EGFR protein is comprised of seven structural protein domains and a putative signal peptide comprised of amino acids 1-24. There are four extracellular domains (I, II, III, IV) that together comprise 621 amino acids and the first 15 exons (exon #15 spans extracellular domain IV and the transmembrane domain).

EGFR ectodomain mutations have been identified in exon 12 which encodes amino acids 433-499. Shown in FIG. 17B is a table of representative mutations that have been identified in clinical and/or preclinical studies and reported in the literature. This region spans the end portion of extracellular domain III and the beginning portion of domain IV.

### Example 18

### MM-151 Blocks the Activation of EGFR by Both Low and High Affinity EGFR Ligands in LIM1215 Cells Expressing or Over-Expressing the Representative S492R Ectodomain Mutation

To demonstrate that the growth inhibition and clonal expansion inhibition observed in the previous Examples 15 and 16 is due to blockage of the EGFR signaling, a phospho-EGFR inhibition assay was performed with cells expressing wild type EGFR (LIM1215 (#2) EGFR S492 wild type), overexpressing the EGFR S492R mutation in addition to wild type EGFR (LIMI1215 (#2) EGFR S492R), and expressing the EGFR S492R mutation via gene editing (LIM1215 (#2) EGFR S492R KI). Cells were treated for 24 hours with cetuximab, the two-antibody mixture of sym-992 and sym-1024 (“Sym004”) and MM-151 followed by stimulation with 8 nM EGF or AREG ligand for 10 minutes. Immunoblotting was performed using antibodies to phospho-EGFR (“pEGFR”) and actin as a loading control.

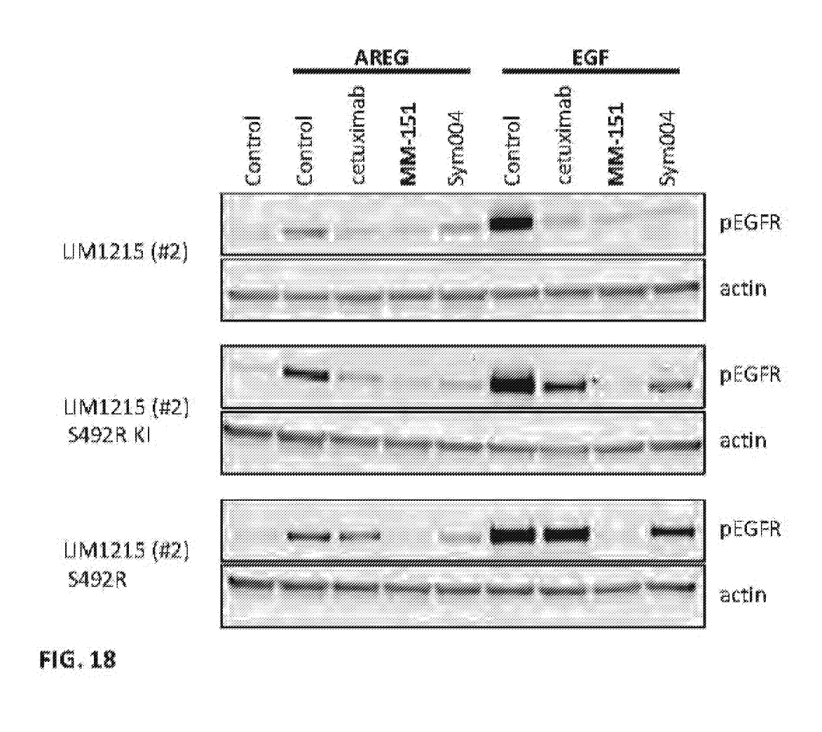

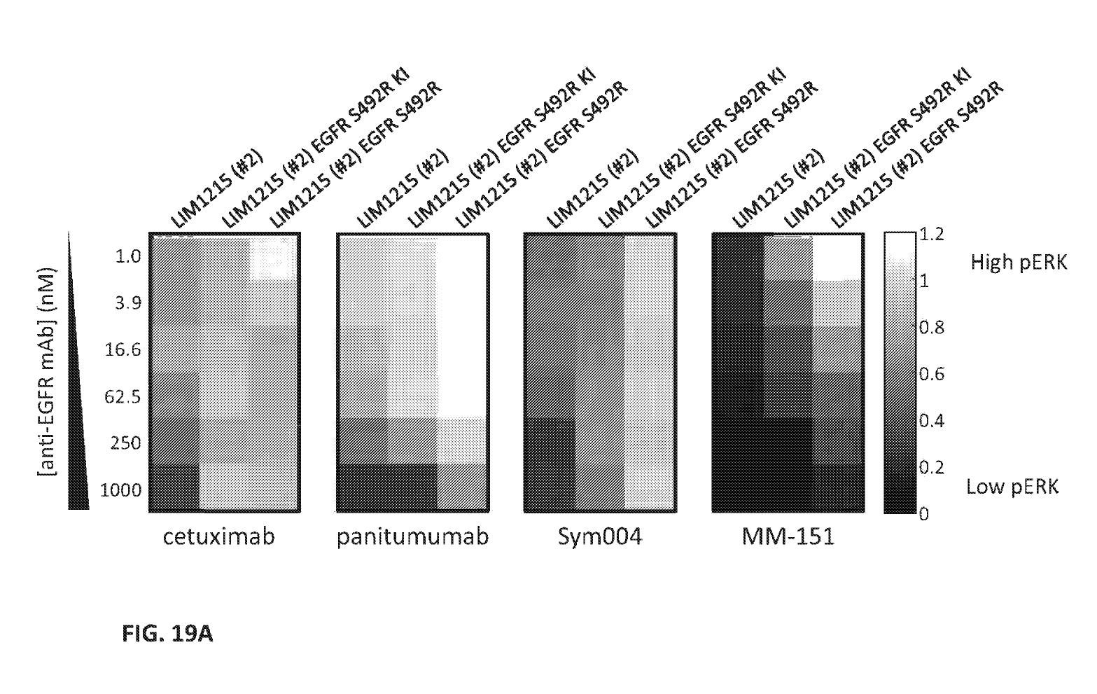

As shown in FIG. 18, MM-151 is able to inhibit EGFR activation by AREG and EGF ligands in the LIM1215 (#2) control cells and cells expressing or over-expressing the EGFR S492R ectodomain mutation. In contrast, while cetuximab and Sym004 are able to inhibit EGFR activation in cells expressing wild type EGFR, they elicited only partial or ineffective inhibition of phospho-EGFR stimulated by AREG and EGF in the cells expressing or over-expressing the EGFR S492R mutation.

### Example 19

### MM-151 Blocks the Activation of ERK by Both Low and High Affinity EGFR Ligands in LIM1215 Cells Expressing or Over-Expressing the Representative S492R Ectodomain Mutation

To demonstrate that the growth inhibition and clonal expansion inhibition observed in the previous Examples 15 and 16 is due to blockage of the cell signaling downstream of EGFR, a phospho-ERK inhibition assay was performed with cells expressing wild type EGFR (LIM1215 (#2) EGFR S492 wild type), overexpressing the EGFR S492R mutation in addition to wild type EGFR (LIM1215 (#2) EGFR S492R), and expressing the EGFR S492R mutation via gene editing (LIM1215 (#2) EGFR S492R KI). Cells were treated for two hours with cetuximab, the two-antibody mixture of sym-992 and sym-1024 (“Sym004”) and MM-151 followed by stimulation with 8 nM EGF or AREG ligand for 10 minutes. An ELISA experiment was performed to measure activation of ERK (phospho-ERK).

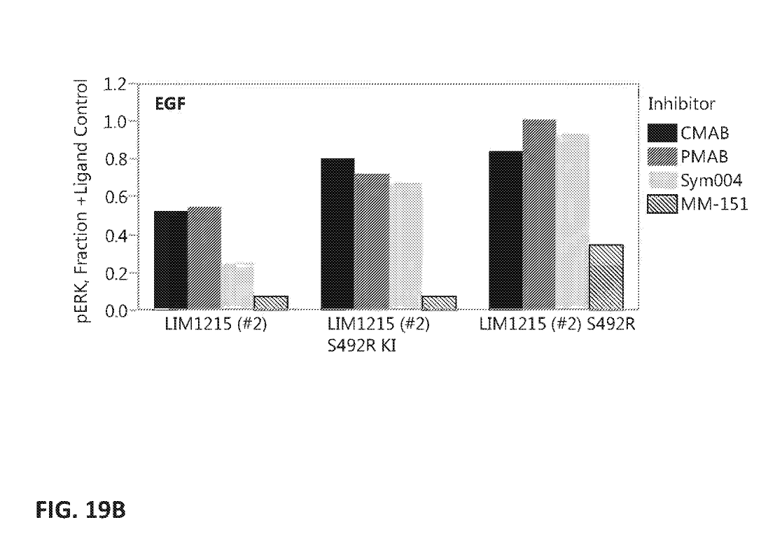

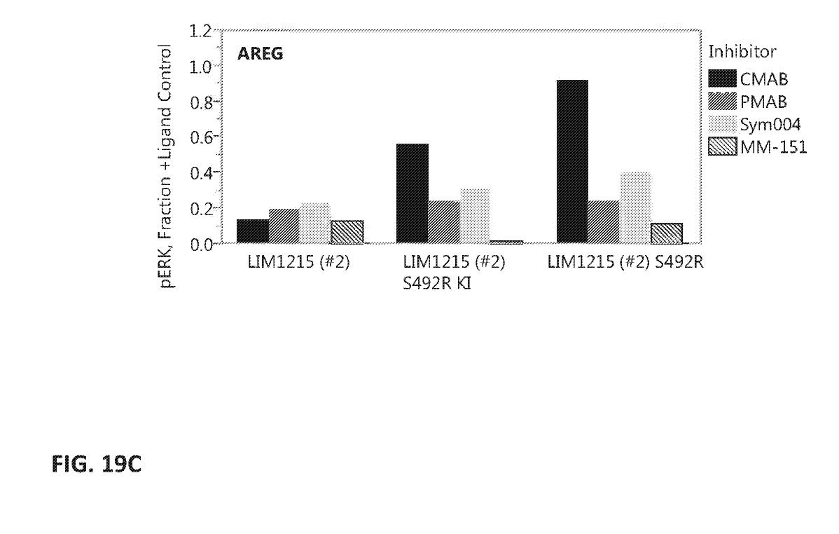

As shown in FIG. 19A, MM-151 is able to inhibit ERK activation by EGF in the LIM1215 (#2) control cells and cells expressing or over-expressing the EGFR S492R ectodomain mutation. In contrast, cetuximab and Sym004 only inhibits phospho-ERK in the control cells and panitumumab only inhibits phospho-ERK in the control cells and engineered LIM1215 (#2) EGFR S492R KI cells. Cetuximab, panitumumab, and Sym004 are ineffective at inhibiting phospho-ERK in cells over-expressing the EGFR S492R mutation.

FIGS. 19B and 19C show inhibition of phospho-ERK by treatment with drug at a concentration of 250 nM of drug followed by stimulation with 8 nM of EGF or AREG ligand for 10 minutes. MM-151 is able to inhibit phospho-ERK activated by AREG and EGF ligands in the three cell lines. In contrast, cetuximab and panitumumab are not effective at inhibiting phospho-ERK stimulated by EGF ligand and Sym004 provides moderate inhibition only in the LIM1215 (#2) control cells. Cetuximab, panitumumab, and Sym004 are able to inhibit phospho-ERK activated by AREG ligand in the LIM1215 (#2) control cells, but provide ineffective or partial inhibition in cells expressing or over-expressing the EGFR S492R mutations.

### Example 20

### The Growth of LIM1215 Cells Stimulated by Low or High-Affinity EGFR Ligand Expressing or Overexpressing the EGFR S492R Ectodomain Mutant is Inhibited by MM-151, Resistant to Cetuximab, and Only Selectively Inhibited by Panitumumab and Sym004

To demonstrate that MM-151 inhibits both ligand-independent and ligand-stimulated cell growth in cells harboring expression of the representative EGFR S492R mutation, a cell growth assay was performed with cells expressing wild type EGFR (LIM1215 (#2) EGFR S492 wild type), overexpressing the EGFR S492R mutation in addition to wild type EGFR (LIM1215 (#2) EGFR S492R), and expressing the EGFR S492R mutation via gene editing (LIM1215 (#2) EGFR S492R KI).

Cells were incubated for three days on a live-cell imager with increasing concentrations of cetuximab (“CMAB”, dark grey line with triangle marker), panitumumab (“PMAB”; light grey line with diamond marker), the two-antibody mixture of sym-992 and sym-1024 (“Sym004”, dark grey line with circle marker) and MM-151 (black line with square marker) in the presence of 8 nM EGF or AREG ligand. The confluence of the well was assessed at two-hour intervals over the 72 hour experiment and used to calculate the cell growth rate for under each condition.

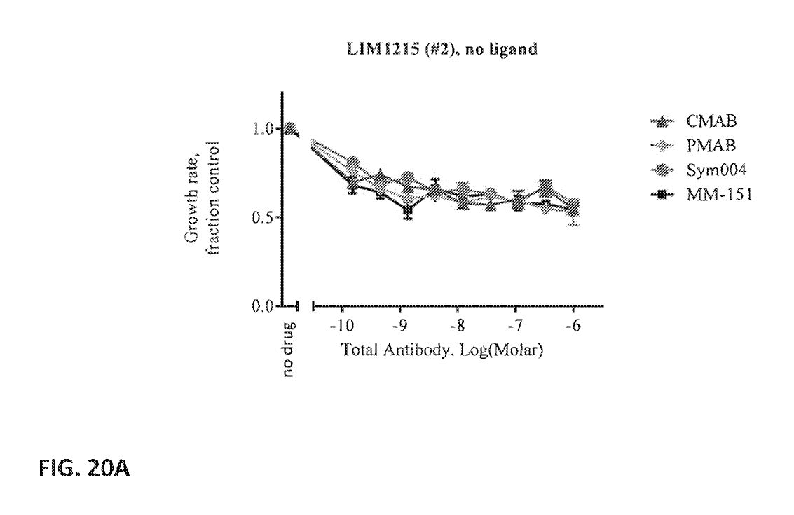

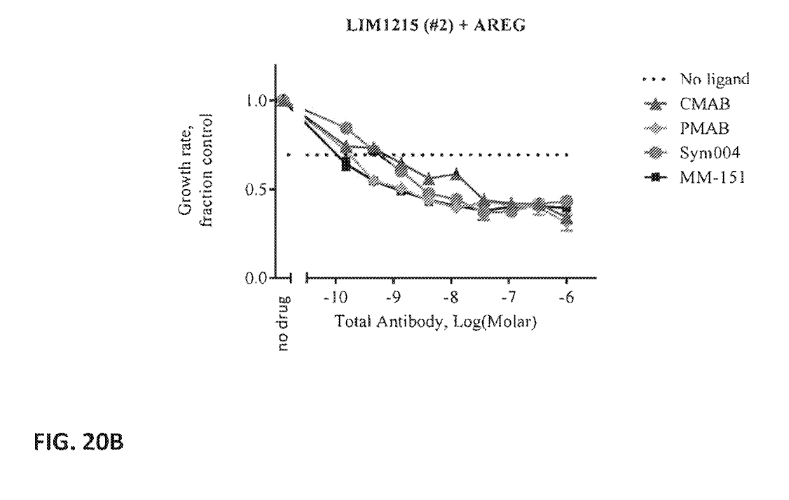

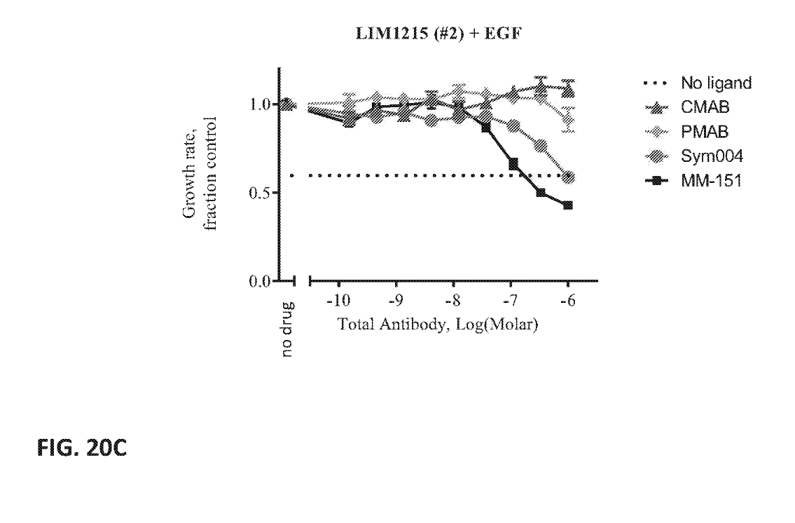

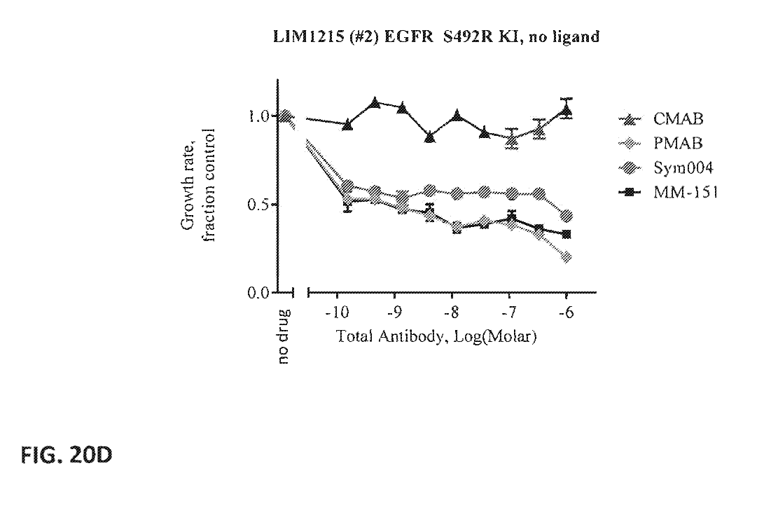

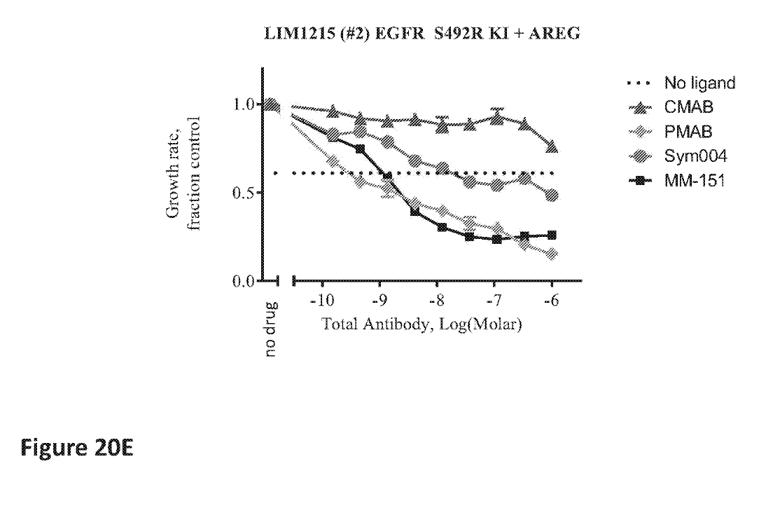

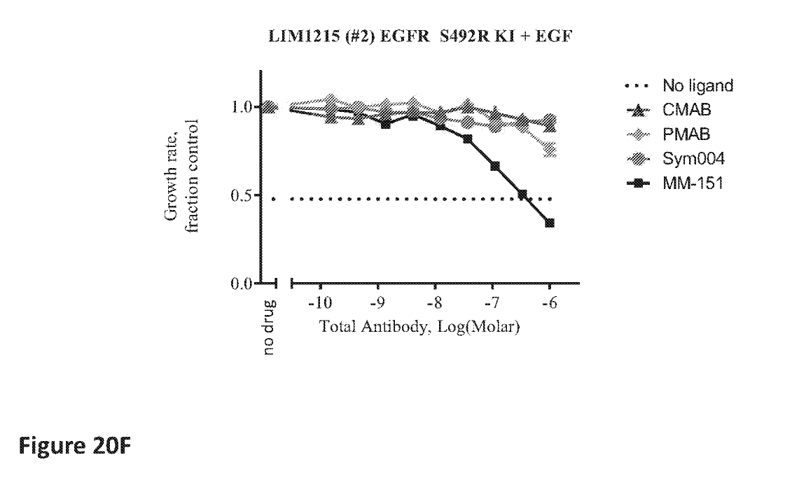

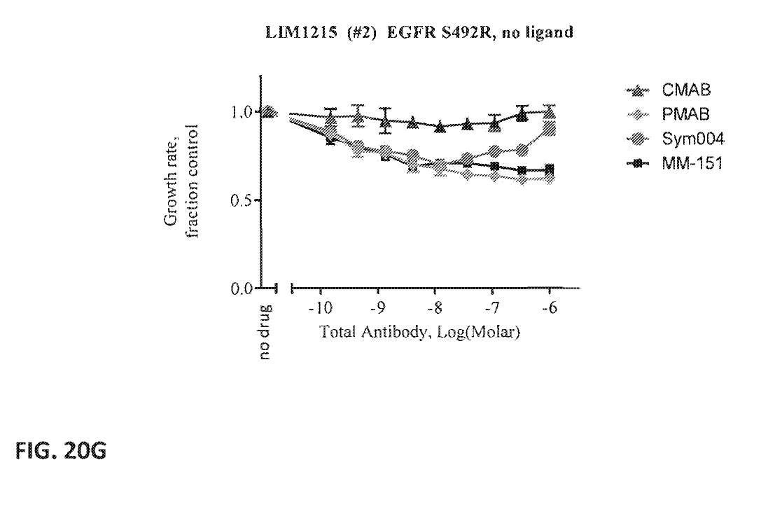

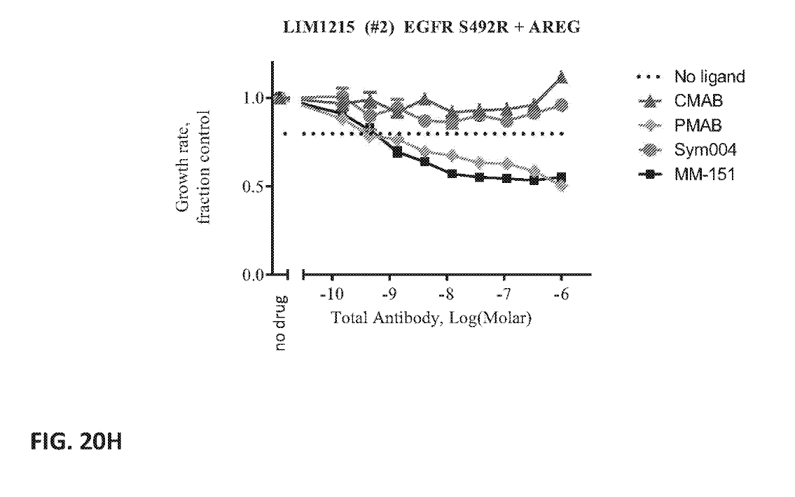

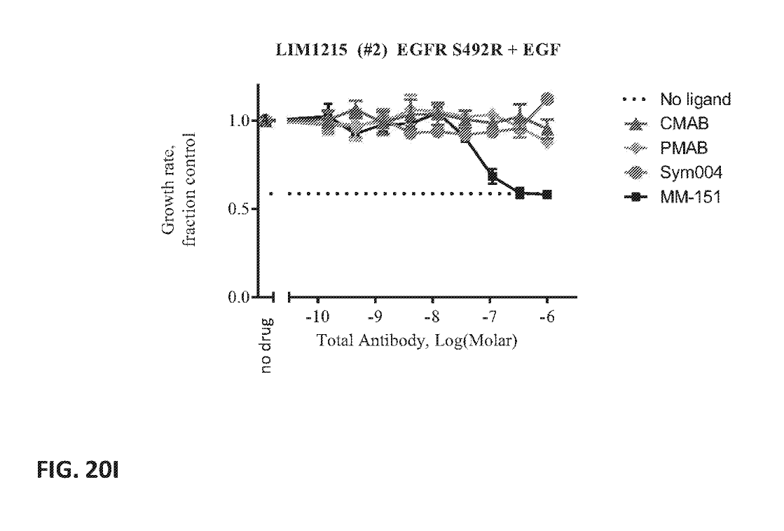

As shown in FIGS. 20A and 20B, the cell growth rate of the LIM1215 (#2) cells without ligand and stimulated with the low-affinity EGFR ligand AREG is inhibited by all four drugs. As shown in FIG. 20C, however, cetuximab and panitumumab are unable to inhibit cell growth stimulated by the high-affinity EGFR ligand EGF in this cell line while Sym004 elicits partial inhibition (to the no ligand control). In contrast, MM-151 elicits strong inhibition (below the no ligand control).

A second experiment was performed with the LIM1215 (#2) EGFR S492R KI cell line. As shown in FIGS. 20D-20F, cells expressing the EGFR S492R mutation are resistant to cetuximab when grown in media alone or with exogenous AREG or EGF. As shown in FIG. 20E, stimulation with low-affinity EGFR ligand AREG reduces the inhibition provided by Sym004 (as compared to the parental cell line in FIG. 20B) but retains sensitivity to panitumumab and MM-151. As shown in FIG. 20F, cell growth stimulated by EGF is only inhibited by MM-151.

A third experiment was performed with the LIM1215 (#2) EGFR S492R cell line (FIGS. 20G-I). Cells overexpressing the EGFR S492R mutation are resistant to cetuximab when grown in media alone or with exogenous AREG or EGF. Sym004 provides partial inhibition of cell growth in media alone, but no inhibition of ligand-dependent cell growth (AREG or EGF). Panitumumab inhibits cell growth in media alone and in the presence of AREG, but not in the presence of EGF. In contrast, MM-151 inhibits both ligand-independent and ligand-dependent cell growth in cells that overexpress the representative EGFR S492R mutation.

SEQUENCE SUMMARY

| P1X V_{H} | SYAIS | SEQ ID |
|---|---|---|
| CDR1 | | NO: 1 |
| P1X V_{H} | IIPIFGTVNY | SEQ ID |
| CDR2 | | NO: 2 |
| P1X V_{H} | DPSVNL | SEQ ID |
| CDR3 | | NO: 3 |
| P1X V_{L} | QSISSWWA | SEQ ID |
| CDR1 | | NO: 4 |
| P1X V_{L} | DASSL | SEQ ID |
| CDR2 | | NO: 5 |
| P1X V_{L} | QQYHAHP | SEQ ID |
| CDR3 | | NO: 6 |
| P2X V_{H} | SYAIS | SEQ ID |
| CDR1 | | NO: 7 |
| P2X V_{H} | IIPIFGAANP | SEQ ID |
| CDR2 | | NO: 8 |
| P2X V_{H} | MGRGKV | SEQ ID |
| CDR3 | | NO: 9 |
| P2X V_{L} | QSVLYSPNNKNYLA | SEQ ID |
| CDR1 | | NO: 10 |
| P2X V_{L} | WASTR | SEQ ID |
| CDR2 | | NO: 11 |
| P2X V_{L} | QQYYGSP | SEQ ID |
| CDR3 | | NO: 12 |
| P3X V_{H} | SYGIN | SEQ ID |
| CDR1 | | NO: 13 |
| P3X V_{H} | ISAYNGNTYY | SEQ ID |
| CDR2 | | NO: 14 |
| P3X V_{H} | DLGGYGSGS | SEQ ID |
| CDR3 | | NO: 15 |
| P3X V_{L} | QSVSSNLA | SEQ ID |
| CDR1 | | NO: 16 |
| P3X V_{L} | GASTR | SEQ ID |
| CDR2 | | NO: 17 |
| P3X V_{L} | QDYRTWPR | SEQ ID |
| CDR3 | | NO: 18 |
| P1X V_{H} | MGFGLSWLFLVAILKGVQCQ | SEQ ID |
| | VQLVQSGAEVKKPGSSVKVS | NO: 19 |
| | CKASGGTFSSYAISWVRQAP | |
| | GQGLEWMGSIIPIFGTVNYA | |
| | QKFQGRVTITADESTSTAYM | |
| | ELSSLRSEDTAVYYCARDPS | |
| | VNLYWYFDLWGRGTLVTVSS | |
| P1X V_{L} | MGTPAQLLFLLLLWLPDTTG | SEQ ID |
| | DIQMTQSPSTLSASVGDRVT | NO: 20 |
| | ITCRASQSISSWWAWYQQKP | |
| | GKAPKLLIYDASSLESGVPS | |
| | RFSGSGSGTEFTLTISSLQP | |
| | DDFATYYCQQYHAHPTTFGG | |
| | GTKVEIK | |
| P2X V_{H} | MGFGLSWLFLVAILKGVQCQ | SEQ ID |
| | VQLVQSGAEVKKPGSSVKVS | NO: 21 |
| | CKASGGTFGSYAISWVRQAP | |
| | GQGLEWMGSIIPIFGAANPA | |
| | QKSQGRVTITADESTSTAYM | |
| | ELSSLRSEDTAVYYCAKMGR | |
| | GKVAFDIWGQGTMVTVSS | |
| P2X V_{L} | MGTPAQLLFLLLLWLPDTTG | SEQ ID |
| | DIVMTQSPDSLAVSLGERAT | NO: 22 |
| | INCKSSQSVLYSPNNKNYLA | |
| | WYQQKPGQPPKLLIYWASTR | |
| | ESGVPDRFSGSGSGTDFTLT | |
| | ISSLQAEDVAVYYCQQYYGS | |
| | PITFGGGTKVEIK | |
| P3X V_{H} | MGFGLSWLFLVAILKGVQCQ | SEQ ID |
| | VQLVQSGAEVKKPGASVKVS | NO: 23 |
| | CKASGYAFTSYGINWVRQAP | |
| | GQGLEWMGWISAYNGNTYYA | |
| | QKLRGRVTMTTDTSTSTAYM | |
| | ELRSLRSDDTAVYYCARDLG | |
| | GYGSGSVPFDPWGQGTLVTV | |
| | SS | |
| P3X V_{L} | MGTPAQLLFLLLLWLPDTTG | SEQ ID |
| | EIVMTQSPATLSVSPGERAT | NO: 24 |
| | LSCRASQSVSSNLAWYQQKP | |
| | GQAPRLLIYGASTRATGIPA | |
| | RFSGSGSGTEFTLTISSLQS | |
| | EDFAVYYCQDYRTWPRRVFG | |
| | GGTKVEIK | |
| 25E heavy | QVQLVQSGAEVKKPGSSVKV | SEQ ID |
| chain | SCKASGGTFSSYAISWVRQA | NO: 25 |
| | PGQGLEWMGSIIPIFGTVNY | |
| | AQKFQGRVTITADESTSTAY | |
| | MELSSLRSEDTAVYYCARDP | |
| | SVNLYWYFDLWGRGTLVTVS | |
| | SASTKGPSVFPLAPSSKSTS | |
| | GGTAALGCLVKDYFPEPVTV | |
| | SWNSGALTSGVHTFPAVLQS | |
| | SGLYSLSSVVTVPSSSLGTQ | |
| | TYICNVNHKPSNTKVDKKVE | |
| | PKSCDKTHTCPPCPAPELLG | |
| | GPSVFLFPPKPKDTLMISRT | |
| | PEVTCVVVDVSHEDPEVKFN | |
| | WYVDGVEVHNAKTKPREEQY | |
| | NSTYRVVSVLTVLHQDWLNG | |
| | KEYKCKVSNKALPAPIEKTI | |
| | SKAKGQPREPQVYTLPPSRE | |
| | EMTKNQVSLTCLVKGFYPSD | |
| | IAVEWESNGQPENNYKTTPP | |
| | VLDSDGSFFLYSKLTVDKSR | |
| | WQQGNVFSCSVMHEALHNHY | |
| | TQKSLSLSPGK | |
| 25E light | DIQMTQSPSTLSASVGDRVT | SEQ ID |
| chain | ITCRASQSISSWWAWYQQKP | NO: 26 |
| | GKAPKLLIYDASSLESGVPS | |
| | RFSGSGSGTEFTLTISSLQP | |
| | DDFATYYCQQYHAHPTTFGG | |
| | GTKVEIKRTVAAPSVFIFPP | |
| | SDEQLKSGTASVVCLLNNFY | |
| | PREAKVQWKVDNALQSGNSQ | |
| | ESVTEQDSKDSTYSLSSTLT | |
| | LSKADYEKHKVYACEVTHQG | |
| | LSSPVTKSFNRGEC | |
| EGFR | MRPSGTAGAALLALLAALCP | SEQ ID |
| ECD | ASRALEEKKVCQGTSNKLTQ | NO: 27 |
| | LGTFEDHFLSLQRMFNNCEV | |
| | VLGNLEITYVQRNYDLSFLK | |
| | TIQEVAGYVLIALNTVERIP | |
| | LENLQIIRGNMYYENSYALA | |
| | VLSNYDANKTGLKELPMRNL | |
| | QEILHGAVRFSNNPALCNVE | |
| | SIQWRDIVSSDFLSNMSMDF | |
| | QNHLGSCQKCDPSCPNGSCW | |
| | GAGEENCQKLTKIICAQQCS | |
| | GRCRGKSPSDCCHNQCAAGC | |
| | TGPRESDCLVCRKFRDEATC | |
| | KDTCPPLMLYNPTTYQMDVN | |
| | PEGKYSFGATCVKKCPRNYV | |
| | VTDHGSCVRACGADSYEMEE | |
| | DGVRKCKKCEGPCRKVCNGI | |
| | GIGEFKDSLSINATNIKHFK | |
| | NCTSISGDLHILPVAFRGDS | |
| | FTHTPPLDPQELDILKTVKE | |
| | ITGFLLIQAWPENRTDLHAF | |
| | ENLEIIRGRTKQHGQFSLAV | |
| | VSLNITSLGLRSLKEISDGD | |
| | VIISGNKNLCYANTINWKKL | |
| | FGTSGQKTKIISNRGENSCK | |
| | ATGQVCHALCSPEGCWGPEP | |
| | RDCVSCRNVSRGRECVDKCN | |
| | LLEGEPREFVENSECIQCHP | |
| | ECLPQAMNITCTGRGPDNCI | |
| | QCAHYIDGPHCVKTCPAGVM | |
| | GENNTLVWKYADAGHVCHLC | |
| | HPNCTYGCTGPGLEGCPTNG | |
| | PKIPSHHHHHH | |
| Neo | 5′ AAGATGGGGGAAAGAAG | SEQ ID |
| screening | AGC 3′ | NO: 28 |
| forward | | |
| primer | | |
| Neo | 5′ GCATGCTCCAGACTGCC | SEQ ID |
| screening | TTG 3′ | NO: 29 |
| reverse | | |
| primer | | |
| Genomic | 5′ AAGATGGGGGAAAGAAG | SEQ ID |
| DNA | AGC 3′ | NO: 30 |
| forward | | |
| primer | | |
| Neomycin | 5′ GCATGCTCCAGACTGCC | SEQ ID |
| reverse | TTG 3′ | NO: 31 |
| primer | | |
| Sequence | cccggggttattaatagtaa | SEQ ID |
| of PLX301 | tcaattacggggtcattagt | NO: 32 |
| plasmid | tcatagcccatatatggagt | |
| | tccgcgttacataacttacg | |
| | gtaaatggcccgcctggctg | |
| | accgcccaacgacccccgcc | |
| | cattgacgtcaataatgacg | |
| | tatgttcccatagtaacgcc | |
| | aatagggactttccattgac | |
| | gtcaatgggtggagtattta | |
| | cggtaaactgcccacttggc | |
| | agtacatcaagtgtatcata | |
| | tgccaagtacgccccctatt | |
| | gacgtcaatgacggtaaatg | |
| | gcccgcctggcattatgccc | |
| | agtacatgaccttatgggac | |
| | tttcctacttggcagtacat | |
| | ctacgtattagtcatcgcta | |
| | ttaccatggtgatgcggttt | |
| | tggcagtacatcaatgggcg | |
| | tggatagcggtttgactcac | |
| | ggggatttccaagtctccac | |
| | cccattgacgtcaatgggag | |
| | tttgttttggcaccaaaatc | |
| | aacgggactttccaaaatgt | |
| | cgtaacaactccgccccatt | |
| | gacgcaaatgggcggtaggc | |
| | gtgtacggtgggaggtctat | |
| | ataagcagagctctctggct | |
| | aactgtcgggatcaacaagt | |
| | ttgtacaaaaaagctgaacg | |
| | agaaacgtaaaatgatataa | |
| | atatcaatatattaaattag | |
| | attttgcataaaaaacagac | |
| | tacataatactgtaaaacac | |
| | aacatatccagtcactatgg | |
| | cggccgcattaggcacccca | |
| | ggctttacactttatgcttc | |
| | cggctcgtataatgtgtgga | |
| | ttttgagttaggatccgtcg | |
| | agattttcaggagctaagga | |
| | agctaaaatggagaaaaaaa | |
| | tcactggatataccaccgtt | |
| | gatatatcccaatggcatcg | |
| | taaagaacattttgaggcat | |
| | ttcagtcagttgctcaatgt | |
| | acctataaccagaccgttca | |
| | gctggatattacggcctttt | |
| | taaagaccgtaaagaaaaat | |
| | aagcacaagttttatccggc | |
| | ctttattcacattcttgccc | |
| | gcctgatgaatgctcatccg | |
| | gaattccgtatggcaatgaa | |
| | agacggtgagctggtgatat | |
| | gggatagtgttcacccttgt | |
| | tacaccgttttccatgagca | |
| | aactgaaacgttttcatcgc | |
| | tctggagtgaataccacgac | |
| | gatttccggcagtttctaca | |
| | catatattcgcaagatgtgg | |
| | cgtgttacggtgaaaacctg | |
| | gcctatttccctaaagggtt | |
| | tattgagaatatgtttttcg | |
| | tctcagccaatccctgggtg | |
| | agtttcaccagttttgattt | |
| | aaacgtggccaatatggaca | |
| | acttcttcgcccccgttttc | |
| | accatgggcaaatattatac | |
| | gcaaggcgacaaggtgctga | |
| | tgccgctggcgattcaggtt | |
| | catcatgccgtttgtgatgg | |
| | cttccatgtcggcagaatgc | |
| | ttaatgaattacaacagtac | |
| | tgcgatgagtggcagggcgg | |
| | ggcgtaaagatctggatccg | |
| | gcttactaaaagccagataa | |
| | cagtatgcgtatttgcgcgc | |
| | tgatttttgcggtataagaa | |
| | tatatactgatatgtatacc | |
| | cgaagtatgtcaaaaagagg | |
| | tatgctatgaagcagcgtat | |
| | tacagtgacagttgacagcg | |
| | acagctatcagttgctcaag | |
| | gcatatatgatgtcaatatc | |
| | tccggtctggtaagcacaac | |
| | catgcagaatgaagcccgtc | |
| | gtctgcgtgccgaacgctgg | |
| | aaagcggaaaatcaggaagg | |
| | gatggctgaggtcgcccggt | |
| | ttattgaaatgaacggctct | |
| | tttgctgacgagaacagggg | |
| | ctggtgaaatgcagtttaag | |
| | gtttacacctataaaagaga | |
| | gagccgttatcgtctgtttg | |
| | tggatgtacagagtgatatt | |
| | attgacacgcccgggcgacg | |
| | gatggtgatccccctggcca | |
| | gtgcacgtctgctgtcagat | |
| | aaagtctcccgtgaacttta | |
| | cccggtggtgcatatcgggg | |
| | atgaaagctggcgcatgatg | |
| | accaccgatatggccagtgt | |
| | gccggtctccgttatcgggg | |
| | aagaagtggctgatctcagc | |
| | caccgcgaaaatgacatcaa | |
| | aaacgccattaacctgatgt | |
| | tctggggaatataaatgtca | |
| | ggctcccttatacacagcca | |
| | gtctgcaggtcgaccatagt | |
| | gactggatatgttgtgtttt | |
| | acagtattatgtagtctgtt | |
| | ttttatgcaaaatctaattt | |
| | aatatattgatatttatatc | |
| | attttacgtttctcgttcag | |
| | ctttcttgtacaaagtggtt | |
| | tagtaatgagctagcgctaa | |
| | ccggtggcgcgttaagtcga | |
| | caatcaacctctggattaca | |
| | aaatttgtgaaagattgact | |
| | ggtattcttaactatgttgc | |
| | tccttttacgctatgtggat | |
| | acgctgctttaatgcctttg | |
| | tatcatgctattgcttcccg | |
| | tatggctttcattttctcct | |
| | ccttgtataaatcctggttg | |
| | ctgtctctttatgaggagtt | |
| | gtggcccgttgtcaggcaac | |
| | gtggcgtggtgtgcactgtg | |
| | tttgctgacgcaacccccac | |
| | tggttggggcattgccacca | |
| | cctgtcagctcctttccggg | |
| | actttcgctttccccctccc | |
| | tattgccacggcggaactca | |
| | tcgccgcctgccttgcccgc | |
| | tgctggacaggggctcggct | |
| | gttgggcactgacaattccg | |
| | tggtgttgtcggggaaatca | |
| | tcgtcctttccttggctgct | |
| | cgcctgtgttgccacctgga | |
| | ttctgcgcgggacgtccttc | |
| | tgctacgtcccttcggccct | |
| | caatccagcggaccttcctt | |
| | cccgcggcctgctgccggct | |
| | ctgcggcctcttccgcgtct | |
| | tcgccttcgccctcagacga | |
| | gtcggatctccctttgggcc | |
| | gcctccccgcgtcgacttta | |
| | agaccaatgacttacaaggc | |
| | agctgtagatcttagccact | |
| | ttttaaaagaaaagggggga | |
| | ctggaagggctaattcactc | |
| | ccaacgaagacaagatctgc | |
| | tttttgcttgtactgggtct | |
| | ctctggttagaccagatctg | |
| | agcctgggagctctctggct | |
| | aactagggaacccactgctt | |
| | aagcctcaataaagcttgcc | |
| | ttgagtgcttcaagtagtgt | |
| | gtgcccgtctgttgtgtgac | |
| | tctggtaactagagatccct | |
| | cagacccttttagtcagtgt | |
| | ggaaaatctctagcagtacg | |
| | tatagtagttcatgtcatct | |
| | tattattcagtatttataac | |
| | ttgcaaagaaatgaatatca | |
| | gagagtgagaggaacttgtt | |
| | tattgcagcttataatggtt | |
| | acaaataaagcaatagcatc | |
| | acaaatttcacaaataaagc | |
| | atttttttcactgcattcta | |
| | gttgtggtttgtccaaactc | |
| | atcaatgtatcttatcatgt | |
| | ctggctctagctatcccgcc | |
| | cctaactccgcccatcccgc | |
| | ccctaactccgcccagttcc | |
| | gcccattctccgccccatgg | |
| | ctgactaattttttttattt | |
| | atgcagaggccgaggccgcc | |
| | tcggcctctgagctattcca | |
| | gaagtagtgaggaggctttt | |
| | ttggaggcctagggacgtac | |
| | ccaattcgccctatagtgag | |
| | tcgtattacgcgcgctcact | |
| | ggccgtcgttttacaacgtc | |
| | gtgactgggaaaaccctggc | |
| | gttacccaacttaatcgcct | |
| | tgcagcacatccccctttcg | |
| | ccagctggcgtaatagcgaa | |
| | gaggcccgcaccgatcgccc | |
| | ttcccaacagttgcgcagcc | |
| | tgaatggcgaatgggacgcg | |
| | ccctgtagcggcgcattaag | |
| | cgcggcgggtgtggtggtta | |
| | cgcgcagcgtgaccgctaca | |
| | cttgccagcgccctagcgcc | |
| | cgctcctttcgctttcttcc | |
| | cttcctttctcgccacgttc | |
| | gccggctttccccgtcaagc | |
| | tctaaatcgggggctccctt | |
| | tagggttccgatttagtgct | |
| | ttacggcacctcgaccccaa | |
| | aaaacttgattagggtgatg | |
| | gttcacgtagtgggccatcg | |
| | ccctgatagacggtttttcg | |
| | ccctttgacgttggagtcca | |
| | cgttctttaatagtggactc | |
| | ttgttccaaactggaacaac | |
| | actcaaccctatctcggtct | |
| | attcttttgatttataaggg | |
| | attttgccgatttcggccta | |
| | ttggttaaaaaatgagctga | |
| | tttaacaaaaatttaacgcg | |
| | aattttaacaaaatattaac | |
| | gcttacaatttaggtggcac | |
| | ttttcggggaaatgtgcgcg | |
| | gaacccctatttgtttattt | |
| | ttctaaatacattcaaatat | |
| | gtatccgctcatgagacaat | |
| | aaccctgataaatgcttcaa | |
| | taatattgaaaaaggaagag | |
| | tatgagtattcaacatttcc | |
| | gtgtcgcccttattcccttt | |
| | tttgcggcattttgccttcc | |
| | tgtttttgctcacccagaaa | |
| | cgctggtgaaagtaaaagat | |
| | gctgaagatcagttgggtgc | |
| | acgagtgggttacatcgaac | |
| | tggatctcaacagcggtaag | |
| | atccttgagagttttcgccc | |
| | cgaagaacgttttccaatga | |
| | tgagcacttttaaagttctg | |
| | ctatgtggcgcggtattatc | |
| | ccgtattgacgccgggcaag | |
| | agcaactcggtcgccgcata | |
| | cactattctcagaatgactt | |
| | ggttgagtactcaccagtca | |
| | cagaaaagcatcttacggat | |
| | ggcatgacagtaagagaatt | |
| | atgcagtgctgccataacca | |
| | tgagtgataacactgcggcc | |
| | aacttacttctgacaacgat | |
| | cggaggaccgaaggagctaa | |
| | ccgcttttttgcacaacatg | |
| | ggggatcatgtaactcgcct | |
| | tgatcgttgggaaccggagc | |
| | tgaatgaagccataccaaac | |
| | gacgagcgtgacaccacgat | |
| | gcctgtagcaatggcaacaa | |
| | cgttgcgcaaactattaact | |
| | ggcgaactacttactctagc | |
| | ttcccggcaacaattaatag | |
| | actggatggaggcggataaa | |
| | gttgcaggaccacttctgcg | |
| | ctcggcccttccggctggct | |
| | ggtttattgctgataaatct | |
| | ggagccggtgagcgtgggtc | |
| | tcgcggtatcattgcagcac | |
| | tggggccagatggtaagccc | |
| | tcccgtatcgtagttatcta | |
| | cacgacggggagtcaggcaa | |
| | ctatggatgaacgaaataga | |
| | cagatcgctgagataggtgc | |
| | ctcactgattaagcattggt | |
| | aactgtcagaccaagtttac | |
| | tcatatatactttagattga | |
| | tttaaaacttcatttttaat | |
| | ttaaaaggatctaggtgaag | |
| | atcctttttgataatctcat | |
| | gaccaaaatcccttaacgtg | |
| | agttttcgttccactgagcg | |
| | tcagaccccgtagaaaagat | |
| | caaaggatcttcttgagatc | |
| | ctttttttctgcgcgtaatc | |
| | tgctgcttgcaaacaaaaaa | |
| | accaccgctaccagcggtgg | |
| | tttgtttgccggatcaagag | |
| | ctaccaactctttttccgaa | |
| | ggtaactggcttcagcagag | |
| | cgcagataccaaatactgtt | |
| | cttctagtgtagccgtagtt | |
| | aggccaccacttcaagaact | |
| | ctgtagcaccgcctacatac | |
| | ctcgctctgctaatcctgtt | |
| | accagtggctgctgccagtg | |
| | gcgataagtcgtgtcttacc | |
| | gggttggactcaagacgata | |
| | gttaccggataaggcgcagc | |
| | ggtcgggctgaacggggggt | |
| | tcgtgcacacagcccagctt | |
| | ggagcgaacgacctacaccg | |
| | aactgagatacctacagcgt | |
| | gagctatgagaaagcgccac | |
| | gcttcccgaagggagaaagg | |
| | cggacaggtatccggtaagc | |
| | ggcagggtcggaacaggaga | |
| | gcgcacgagggagcttccag | |
| | ggggaaacgcctggtatctt | |
| | tatagtcctgtcgggtttcg | |
| | ccacctctgacttgagcgtc | |
| | gatttttgtgatgctcgtca | |
| | ggggggcggagcctatggaa | |
| | aaacgccagcaacgcggcct | |
| | ttttacggttcctggccttt | |
| | tgctggccttttgctcacat | |
| | gttctttcctgcgttatccc | |
| | ctgattctgtggataaccgt | |
| | attaccgcctttgagtgagc | |
| | tgataccgctcgccgcagcc | |
| | gaacgaccgagcgcagcgag | |
| | tcagtgagcgaggaagcgga | |
| | agagcgcccaatacgcaaac | |
| | cgcctctccccgcgcgttgg | |
| | ccgattcattaatgcagctg | |
| | gcacgacaggtttcccgact | |
| | ggaaagcgggcagtgagcgc | |
| | aacgcaattaatgtgagtta | |
| | gctcactcattaggcacccc | |
| | aggctttacactttatgctt | |
| | ccggctcgtatgttgtgtgg | |
| | aattgtgagcggataacaat | |
| | ttcacacaggaaacagctat | |
| | gaccatgattacgccaagcg | |
| | cgcaattaaccctcactaaa | |
| | gggaacaaaagctggagctg | |
| | caagcttaatgtagtcttat | |
| | gcaatactcttgtagtcttg | |
| | caacatggtaacgatgagtt | |
| | agcaacatgccttacaagga | |
| | gagaaaaagcaccgtgcatg | |
| | ccgattggtggaagtaaggt | |
| | ggtacgatcgtgccttatta | |
| | ggaaggcaacagacgggtct | |
| | gacatggattggacgaacca | |
| | ctgaattgccgcattgcaga | |
| | gatattgtatttaagtgcct | |
| | agctcgatacataaacgggt | |
| | ctctctggttagaccagatc | |
| | tgagcctgggagctctctgg | |
| | ctaactagggaacccactgc | |
| | ttaagcctcaataaagcttg | |
| | ccttgagtgcttcaagtagt | |
| | gtgtgcccgtctgttgtgtg | |
| | actctggtaactagagatcc | |
| | ctcagacccttttagtcagt | |
| | gtggaaaatctctagcagtg | |
| | gcgcccgaacagggacttga | |
| | aagcgaaagggaaaccagag | |
| | gagctctctcgacgcaggac | |
| | tcggcttgctgaagcgcgca | |
| | cggcaagaggcgaggggcgg | |
| | cgactggtgagtacgccaaa | |
| | aattttgactagcggaggct | |
| | agaaggagagagatgggtgc | |
| | gagagcgtcagtattaagcg | |
| | ggggagaattagatcgcgat | |
| | gggaaaaaattcggttaagg | |
| | ccagggggaaagaaaaaata | |
| | taaattaaaacatatagtat | |
| | gggcaagcagggagctagaa | |
| | cgattcgcagttaatcctgg | |
| | cctgttagaaacatcagaag | |
| | gctgtagacaaatactggga | |
| | cagctacaaccatcccttca | |
| | gacaggatcagaagaactta | |
| | gatcattatataatacagta | |
| | gcaaccctctattgtgtgca | |
| | tcaaaggatagagataaaag | |
| | acaccaaggaagctttagac | |
| | aagatagaggaagagcaaaa | |
| | caaaagtaagaccaccgcac | |
| | agcaagcggccgctgatctt | |
| | cagacctggaggaggagata | |
| | tgagggacaattggagaagt | |
| | gaattatataaatataaagt | |
| | agtaaaaattgaaccattag | |
| | gagtagcacccaccaaggca | |
| | aagagaagagtggtgcagag | |
| | agaaaaaagagcagtgggaa | |
| | taggagctttgttccttggg | |
| | ttcttgggagcagcaggaag | |
| | cactatgggcgcagcgtcaa | |
| | tgacgctgacggtacaggcc | |
| | agacaattattgtctggtat | |
| | agtgcagcagcagaacaatt | |
| | tgctgagggctattgaggcg | |
| | caacagcatctgttgcaact | |
| | cacagtctggggcatcaagc | |
| | agctccaggcaagaatcctg | |
| | gctgtggaaagatacctaaa | |
| | ggatcaacagctcctgggga | |
| | tttggggttgctctggaaaa | |
| | ctcatttgcaccactgctgt | |
| | gccttggaatgctagttgga | |
| | gtaataaatctctggaacag | |
| | atttggaatcacacgacctg | |
| | gatggagtgggacagagaaa | |
| | ttaacaattacacaagctta | |
| | atacactccttaattgaaga | |
| | atcgcaaaaccagcaagaaa | |
| | agaatgaacaagaattattg | |
| | gaattagataaatgggcaag | |
| | tttgtggaattggtttaaca | |
| | taacaaattggctgtggtat | |
| | ataaaattattcataatgat | |
| | agtaggaggcttggtaggtt | |
| | taagaatagtttttgctgta | |
| | ctttctatagtgaatagagt | |
| | taggcagggatattcaccat | |
| | tatcgtttcagacccacctc | |
| | ccaaccccgaggggaccctt | |
| | gcgccttttccaaggcagcc | |
| | ctgggtttgcgcagggacgc | |
| | ggctgctctgggcgtggttc | |
| | cgggaaacgcagcggcgccg | |
| | accctgggtctcgcacattc | |
| | ttcacgtccgttcgcagcgt | |
| | cacccggatcttcgccgcta | |
| | cccttgtgggccccccggcg | |
| | acgcttcctgctccgcccct | |
| | aagtcgggaaggttccttgc | |
| | ggttcgcggcgtgccggacg | |
| | tgacaaacggaagccgcacg | |
| | tctcactagtaccctcgcag | |
| | acggacagcgccagggagca | |
| | atggcagcgcgccgaccgcg | |
| | atgggctgtggccaatagcg | |
| | gctgctcagcagggcgcgcc | |
| | gagagcagcggccgggaagg | |
| | ggcggtgcgggaggcggggt | |
| | gtggggcggtagtgtgggcc | |
| | ctgttcctgcccgcgcggtg | |
| | ttccgcattctgcaagcctc | |
| | cggagcgcacgtcggcagtc | |
| | ggctccctcgttgaccgaat | |
| | caccgacctctctccccagg | |
| | gggtaccaccggagcttacc | |
| | atgaccgagtacaagcccac | |
| | ggtgcgcctcgccacccgcg | |
| | acgacgtccccagggccgta | |
| | cgcaccctcgccgccgcgtt | |
| | cgccgactaccccgccacgc | |
| | gccacaccgtcgatccggac | |
| | cgccacatcgagcgggtcac | |
| | cgagctgcaagaactcttcc | |
| | tcacgcgcgtcgggctcgac | |
| | atcggcaaggtgtgggtcgc | |
| | ggacgacggcgccgccgtgg | |
| | cggtctggaccacgccggag | |
| | agcgtcgaagcgggggcggt | |
| | gttcgccgagatcggcccgc | |
| | gcatggccgagttgagcggt | |
| | tcccggctggccgcgcagca | |
| | acagatggaaggcctcctgg | |
| | cgccgcaccggcccaaggag | |
| | cccgcgtggttcctggccac | |
| | cgtcggcgtctcgcccgacc | |
| | accagggcaagggtctgggc | |
| | agcgccgtcgtgctccccgg | |
| | agtggaggcggccgagcgcg | |
| | ccggggtgcccgccttcctg | |
| | gagacctccgcgccccgcaa | |
| | cctccccttctacgagcggc | |
| | tcggcttcaccgtcaccgcc | |
| | gacgtcgaggtgcccgaagg | |
| | accgcgcacctggtgcatga | |
| | cccgcaagcccggtgcctga | |
| | gaattctagatcttgagaca | |
| | aatggcagtattcatccaca | |
| | attttaaaagaaaagggggg | |
| | attggggggtacagtgcagg | |
| | ggaaagaatagtagacataa | |
| | tagcaacagacatacaaact | |
| | aaagaattacaaaaacaaat | |
| | tacaaaaattcaaaattttc | |
| | gggtttattacagggacagc | |
| | agagatccactttggcgccg | |
| | gctcgagggggatgaccgag | |
| | tacaagccc | |
| Sequence | atgcgaccctccgggacggc | SEQ ID |
| of EGFR | cggggcagcgctcctggcgc | NO: 33 |
| S492 insert | tgctggctgcgctctgcccg | |
| in the | gcgagtcgggctctggagga | |
| pD2539- | aaagaaagtttgccaaggca | |
| EGFR- | cgagtaacaagctcacgcag | |
| S492R | ttgggcacttttgaagatca | |
| plasmid | ttttctcagcctccagagga | |
| | tgttcaataactgtgaggtg | |
| | gtccttgggaatttggaaat | |
| | tacctatgtgcagaggaatt | |
| | atgatattcatcttaaagac | |
| | catccaggaggtggctggtt | |
| | atgtcctcattgccctcaac | |
| | acagtggagcgaattccttt | |
| | ggaaaacctgcagatcatca | |
| | gaggaaatatgtactacgaa | |
| | aattcctatgccttagcagt | |
| | cttatctaactatgatgcaa | |
| | ataaaaccggactgaaggag | |
| | ctgcccatgagaaatttaca | |
| | ggaaatcctgcatggcgccg | |
| | tgcggttcagcaacaaccct | |
| | gccctgtgcaacgtggagag | |
| | catccagtggcgggacatag | |
| | tcagcagtgactttctcagc | |
| | aacatgtcgatggacttcca | |
| | gaaccacctgggcagctgcc | |
| | aaaagtgtgatccaagctgt | |
| | cccaatgggagctgctgggg | |
| | tgcaggagaggagaactgcc | |
| | agaaactgaccaaaatcatc | |
| | tgtgcccagcagtgctccgg | |
| | gcgctgccgtggcaagtccc | |
| | ccagtgactgctgccacaac | |
| | cagtgtgctgcaggctgcac | |
| | aggcccccgggagagcgact | |
| | gcctggtctgccgcaaattc | |
| | cgagacgaagccacgtgcaa | |
| | ggacacctgccccccactca | |
| | tgctctacaaccccaccacg | |
| | taccagatggatgtgaaccc | |
| | cgagggcaaatacagctttg | |
| | gtgccacctgcgtgaagaag | |
| | tgtccccgtaattatgtggt | |
| | gacagatcacggctcgtgcg | |
| | tccgagcctgtggggccgac | |
| | agctatgagatggaggaaga | |
| | cggcgtccgcaagtgtaaga | |
| | agtgcgaagggccttgccgc | |
| | aaagtgtgtaacggaatagg | |
| | tattggtgaatttaaagact | |
| | cactctccataaatgctacg | |
| | aatattaaacacttcaaaaa | |
| | ctgcacctccatcagtggcg | |
| | atctccacatcctgccggtg | |
| | gcatttaggggtgactcctt | |
| | cacacatactcctcctctgg | |
| | atccacaggaactggatatt | |
| | ctgaaaaccgtaaaggaaat | |
| | cacagggtttttgctgattc | |
| | aggcttggcctgaaaacagg | |
| | acggacctccatgcctttga | |
| | gaacctagaaatcatacgcg | |
| | gcaggaccaagcaacatggt | |
| | cagttttctcttgcagtcgt | |
| | cagcctgaacataacatcct | |
| | tgggattacgctccctcaag | |
| | gagataagtgatggagatgt | |
| | gataatttcaggaaacaaaa | |
| | atttgtgctatgcaaataca | |
| | ataaactggaaaaaactgtt | |
| | tgggacctccggtcagaaaa | |
| | ccaaaattataagaaacaga | |
| | ggtgaaaacagctgcaaggc | |
| | cacaggccaggtctgccatg | |
| | ccttgtgctcccccgagggc | |
| | tgctggggcccggagcccag | |
| | ggactgcgtctcttgccgga | |
| | atgtcagccgaggcagggaa | |
| | tgcgtggacaagtgcaacct | |
| | tctggagggtgagccaaggg | |
| | agtttgtggagaactctgag | |
| | tgcatacagtgccacccaga | |
| | gtgcctgcctcaggccatga | |
| | acatcacctgcacaggacgg | |
| | ggaccagacaactgtatcca | |
| | gtgtgcccactacattgacg | |
| | gcccccactgcgtcaagacc | |
| | tgcccggcaggagtcatggg | |
| | agaaaacaacaccctggtct | |
| | ggaagtacgcagacgccggc | |
| | catgtgtgccacctgtgcca | |
| | tccaaactgcacctacggat | |
| | gcactgggccaggtcttgaa | |
| | ggctgtccaacgaatgggcc | |
| | taagatcccgtccatcgcca | |
| | ctgggatggtgggggccctc | |
| | ctcttgctgctggtggtggc | |
| | cctggggatcggcctcttca | |
| | tgcgaaggcgccacatcgtt | |
| | cggaagcgcacgctgcggag | |
| | gctgctgcaggagagggagc | |
| | ttgtggagcctcttacaccc | |
| | agtggagaagctcccaacca | |
| | agctctcttgaggatcttga | |
| | aggaaactgaattcaaaaag | |
| | atcaaagtgctgggctccgg | |
| | tgcgttcggcacggtgtata | |
| | agggactctggatcccagaa | |
| | ggtgagaaagttaaaattcc | |
| | cgtcgctatcaaggaattaa | |
| | gagaagcaacatctccgaaa | |
| | gccaacaaggaaatcctcga | |
| | tgaagcctacgtgatggcca | |
| | gcgtggacaacccccacgtg | |
| | tgccgcctgctgggcatctg | |
| | cctcacctccaccgtgcagc | |
| | tcatcacgcagctcatgccc | |
| | ttcggctgcctcctggacta | |
| | tgtccgggaacacaaagaca | |
| | atattggctcccagtacctg | |
| | ctcaactggtgtgtgcagat | |
| | cgcaaagggcatgaactact | |
| | tggaggaccgtcgcttggtg | |
| | caccgcgacctggcagccag | |
| | gaacgtactggtgaaaacac | |
| | cgcagcatgtcaagatcaca | |
| | gattttgggctggccaaact | |
| | gctgggtgcggaagagaaag | |
| | aataccatgcagaaggaggc | |
| | aaagtgcctatcaagtggat | |
| | ggcattggaatcaattttac | |
| | acagaatctatacccaccag | |
| | agtgatgtctggagctacgg | |
| | ggtgaccgtttgggagttga | |
| | tgacctttggatccaagcca | |
| | tatgacggaatccctgccag | |
| | cgagatctcctccatcctgg | |
| | agaaaggagaacgcctccct | |
| | cagccacccatatgtaccat | |
| | cgatgtctacatgatcatgg | |
| | tcaagtgctggatgatagac | |
| | gcagatagtcgcccaaagtt | |
| | ccgtgagttgatcatcgaat | |
| | tctccaaaatggcccgcgac | |
| | ccccagcgctaccttgtcat | |
| | tcagggggatgaaagaatgc | |
| | atttgccaagtcctacagac | |
| | tccaacttctaccgtgccct | |
| | gatggatgaagaagacatgg | |
| | acgacgtggtggatgccgac | |
| | gagtacctcatcccacagca | |
| | gggcttcttcagcagcccct | |
| | ccacgtcaagaacgccgtta | |
| | ttatcgagcttgagtgcaac | |
| | cagcaacaattccaccgtgg | |
| | cttgcattgatagaaatggg | |
| | ctgcaaagctgcccgattaa | |
| | agaggattcgttcttgcagc | |
| | gatacagctcagaccccaca | |
| | ggcgccttgactgaggacag | |
| | catagacgacaccttcctcc | |
| | cagtgcctgaatacataaac | |
| | cagtccgttcccaaaaggcc | |
| | cgctggctctgtgcagaatc | |
| | ctgtctatcacaatcagcct | |
| | ctgaaccccgcgcccagccg | |
| | cgacccacactaccaggacc | |
| | cccacagcactgcagtgggc | |
| | aaccccgagtatctcaacac | |
| | tgtccagcccacctgtgtca | |
| | acagcacattcgacagccct | |
| | gcccactgggcccagaaggg | |
| | ttcgcatcagatctcgttag | |
| | acaaccctgactaccagcag | |
| | gacttctttcccaaggaagc | |
| | caagccaaatggcatcttta | |
| | agggctccacagctgaaaat | |
| | gcagaatacctaagggtcgc | |
| | gccacaatcgtccgagttca | |
| | taggtgcttga | |
| Sequence | 5′-caactccataaactaaa | SEQ ID |
| of the | cagaaagcggtgacttactg | NO: 34 |
| single- | cagctgttttcacctctgtt | |
| stranded | tcttataattttggttttct | |
| donor | gaccggaggtcccaaacagt | |
| oligo- | tttttccagtttattgta-3′ | |
| nucleotide | | |
| containing | | |
| the EGFR | | |
| S492R | | |
| mutation | | |
| Sequence | 5′-ccaaacagttttttcca | SEQ ID |
| of the | gtttattgtatttgcatagc | NO: 35 |
| single- | acaaatttttgtttcttgaa | |
| stranded | attatcacatctccatcact | |
| donor | tatctccttgagcgagcgta | |
| oligo- | atcc-3′ | |
| nucleotide | | |
| containing | | |
| the EGFR | | |
| G465R | | |
| mutation | | |
| Mod1a | GGGTTCGCATCAGATCTCGT | SEQ ID |
| primer #1 | TA | NO: 36 |
| Mod1a | TCAAGCACCTATGAACTCGG | SEQ ID |
| primer #2 | AC | NO: 37 |
| Single | AATTTTGGTTTTCTGACCGG | SEQ ID |
| guide RNA | | NO: 38 |
| (sgRNA) | | |
| sequence | | |
| to target | | |
| EGFR | | |
| S492 | | |
| region | | |
| (“S492”) | | |
| Control | ACGGAGGCTAAGCGTCGCAA | SEQ ID |
| non | | NO: 39 |
| specific | | |
| single | | |
| guide RNA | | |
| (sgRNA) | | |
| sequence | | |
| “rg_0111” | | |
| Single | CTCCATCACTTATCTCCTTG | SEQ ID |
| guide RNA | | NO: 40 |
| (sgRNA) | | |
| sequence | | |
| to target | | |
| EGFR | | |
| G465 | | |
| region | | |
| “G465sg1” | | |
| Single | GCTATGCAAATACAATAAAC | SEQ ID |
| guide RNA | | NO: 41 |
| (sgRNA) | | |
| sequence | | |
| to target | | |
| EGFR | | |
| G465 | | |
| region | | |
| “G465sg2” | | |
| Control | ACGGAGGCTAAGCGTCGCAA | SEQ ID |
| non | | NO: 42 |
| specific | | |
| single | | |
| guide RNA | | |
| (sgRNA) | | |
| sequence | | |
| “dual- | | |
| rg0111” | | |
| Control | CGCTTCCGCGGCCCGTTCAA | SEQ ID |
| non | | NO: 43 |
| specific | | |
| single | | |
| guide RNA | | |
| (sgRNA) | | |
| sequence | | |
| “dual-rg- | | |
| 0135” | | |

What is claimed is:

1. A method of treating a human patient having an epidermal growth factor receptor (EGFR) extracellular domain (ECD) mutant cancer having at least one detected mutation in the ECD of EGFR selected from the group consisting of EGFR R451C, S464L, K467T, G465R, G465E, I491M, and S492R, the method comprising administering to the patient an oligoclonal anti-epidermal growth factor receptor (anti-EGFR) antibody combination comprising three monoclonal antibodies, wherein the first monoclonal antibody is P1X, the second monoclonal antibody is P2X, the third monoclonal antibody is P3X, and the oligoclonal antibody combination comprises P1X, P2X and P3X in a 2:2:1 molar ratio.

2. A method of treating an epidermal growth factor receptor (EGFR) extracellular domain (ECD) mutant cancer in a human patient, the method comprising administering to the patient an oligoclonal anti-epidermal growth factor receptor (anti-EGFR) antibody combination, wherein the oligoclonal antibody combination comprises:
a. a first monoclonal antibody comprising heavy chain CDR1, CDR2, and CDR3 sequences of SEQ ID NOs: 1, 2, and 3 respectively, and light chain CDR1, CDR2, and CDR3 sequences of SEQ ID NOs: 4, 5, and 6, respectively;
b. a second monoclonal antibody comprising heavy chain CDR1, CDR2, and CDR3 sequences of SEQ ID NOs: 7, 8, and 9, respectively, and light chain CDR1, CDR2, and CDR3 sequences of SEQ ID NOs: 10, 11 and 12, respectively; and
c. a third monoclonal antibody comprising heavy chain CDR1, CDR2, and CDR3 sequences of SEQ ID NOs: 13, 14, and 15 respectively, and light chain CDR1, CDR2, and CDR3 sequences of SEQ ID NOs: 16, 17, and 18, respectively.

3. The method of claim 2, wherein the first monoclonal antibody is P1X, the second monoclonal antibody is P2X, the third monoclonal antibody is P3X, and the oligoclonal antibody combination comprises P1X, P2X and P3X in a 2:2:1 molar ratio.

4. The method of claim 2, wherein the oligoclonal antibody combination is administered to the patient following treatment with a different anti-EGFR therapy.

5. The method of claim 2, wherein the oligoclonal antibody combination is administered to the patient following (a) treatment with cetuximab or panitumumab or Sym004 and (b) detection of an exon 12 mutation in the ECD region of EGFR in the human patient.

6. The method of claim 3, wherein the oligoclonal antibody combination is administered to the patient following detection of an exon 12 mutation in the ECD region of EGFR in the patient.

7. The method of claim 2, wherein the cancer is head and neck cancer or K-Ras and/or N-Ras wild-type metastatic colorectal cancer.

8. The method of claim 2, wherein the oligoclonal antibody combination is administered to the patient following detection of a mutation in Domain III or Domain IV of the EGFR extracellular domain in the blood of the patient.

9. The method of claim 2, wherein the oligoclonal antibody combination is administered to the patient after obtaining a blood sample from the patient and determining that the blood or urine sample contains circulating DNA with one or more mutations in sequences encoding the extracellular domain of EGFR.

10. The method of claim 2, wherein at least one mutation is detected in the extracellular domain of EGFR in the human patient, wherein the mutation is a protein sequence change, or a DNA or RNA coding region that results in a protein sequence change, selected from the group consisting of EGFR R451C, S464L, K467T, G465R, G465E, I491M, and S492R.

11. The method of claim 2, wherein the cancer is head and neck cancer or Ras wild type colorectal cancer.

12. A method of treating a human patient having an epidermal growth factor receptor (EGFR) extracellular domain (ECD) mutant cancer having a detected mutation in Domain III or Domain IV of the ECD of EGFR, the method comprising administering to the patient a therapeutically effective amount of a MM-151 oligoclonal anti-epidermal growth factor receptor (anti-EGFR) antibody combination, wherein the oligoclonal antibody combination consists of a P1X monoclonal antibody, a P2X monoclonal antibody, and a P3X monoclonal antibody in a 2:2:1 molar ratio.

13. The method of claim 12, wherein the mutation is in exon 12 of the ECD of EGFR in the patient is detected in the patient following treatment with a different anti-EGFR therapy.

14. The method of claim 12, wherein the mutation is in exon 12 of EGFR and the oligoclonal antibody combination is administered to the patient following (a) treatment with cetuximab or panitumumab or Sym004 and (b) detection of an exon 12 mutation in the ECD region of EGFR in the human patient.

15. The method of claim 12, wherein the mutation is in exon 12 of EGFR and the cancer is K-Ras, N-Ras, and/or BRAF wild-type metastatic colorectal cancer or head and neck cancer.

16. The method of claim 12, wherein the mutation is in exon 12 of EGFR and the oligoclonal antibody combination is administered to the patient following detection of a mutation in Domain III of the EGFR extracellular domain in the blood of the patient.

17. The method of claim 12, wherein the mutation is in exon 12 of EGFR and the oligoclonal antibody combination is administered to the patient after obtaining a blood or urine sample from the patient and determining that the sample contains circulating DNA with one or more mutations in sequences encoding the extracellular domain of EGFR.

18. The method of claim 12, wherein the EGFR ECD mutant cancer cells comprise at least one mutation in the extracellular domain of EGFR, wherein the mutation is a protein sequence change, or a DNA or RNA coding region that results in a protein sequence change, selected from the group consisting of EGFR R451C, S464L, K467T, G465R, G465E, I491M, and S492R.

19. The method of claim 12, wherein the cancer is selected from the group consisting of head and neck cancer and colorectal cancer.

20. The method of claim 19, wherein:
a. the cancer is selected from the group consisting of: a Ras-wild type cancer cancer, a BRAF wild type cancer and a Ras-wild type and BRAF wild type cancer;
b. the at least one mutation is detected in a serum or plasma sample of the patient; and
c. the at least one mutation in the extracellular domain of EGFR is a protein sequence change, or a DNA or RNA coding region that results in a protein sequence change, selected from the group consisting of EGFR R451C, S464L, K467T, G465R, G465E I491M, and S492R.


SEQUENCE LISTING (43 sequences)

SEQ ID NO: 1 - protein, 5 aa, Artificial Sequence
  Description of Artificial Sequence Synthetic peptide
SYAIS

SEQ ID NO: 2 - protein, 10 aa, Artificial Sequence
  Description of Artificial Sequence Synthetic peptide
IIPIFGTVNY

SEQ ID NO: 3 - protein, 6 aa, Artificial Sequence
  Description of Artificial Sequence Synthetic peptide
DPSVNL

SEQ ID NO: 4 - protein, 8 aa, Artificial Sequence
  Description of Artificial Sequence Synthetic peptide
QSISSWWA

SEQ ID NO: 5 - protein, 5 aa, Artificial Sequence
  Description of Artificial Sequence Synthetic peptide
DASSL

SEQ ID NO: 6 - protein, 7 aa, Artificial Sequence
  Description of Artificial Sequence Synthetic peptide
QQYHAHP

SEQ ID NO: 7 - protein, 5 aa, Artificial Sequence
  Description of Artificial Sequence Synthetic peptide
SYAIS

SEQ ID NO: 8 - protein, 10 aa, Artificial Sequence
  Description of Artificial Sequence Synthetic peptide
IIPIFGAANP

SEQ ID NO: 9 - protein, 6 aa, Artificial Sequence
  Description of Artificial Sequence Synthetic peptide
MGRGKV

SEQ ID NO: 10 - protein, 14 aa, Artificial Sequence
  Description of Artificial Sequence Synthetic peptide
QSVLYSPNNKNYLA

SEQ ID NO: 11 - protein, 5 aa, Artificial Sequence
  Description of Artificial Sequence Synthetic peptide
WASTR

SEQ ID NO: 12 - protein, 7 aa, Artificial Sequence
  Description of Artificial Sequence Synthetic peptide
QQYYGSP

SEQ ID NO: 13 - protein, 5 aa, Artificial Sequence
  Description of Artificial Sequence Synthetic peptide
SYGIN

SEQ ID NO: 14 - protein, 10 aa, Artificial Sequence
  Description of Artificial Sequence Synthetic peptide
ISAYNGNTYY

SEQ ID NO: 15 - protein, 9 aa, Artificial Sequence
  Description of Artificial Sequence Synthetic peptide
DLGGYGSGS

SEQ ID NO: 16 - protein, 8 aa, Artificial Sequence
  Description of Artificial Sequence Synthetic peptide
QSVSSNLA

SEQ ID NO: 17 - protein, 5 aa, Artificial Sequence
  Description of Artificial Sequence Synthetic peptide
GASTR

SEQ ID NO: 18 - protein, 8 aa, Artificial Sequence
  Description of Artificial Sequence Synthetic peptide
QDYRTWPR

SEQ ID NO: 19 - protein, 140 aa, Artificial Sequence
  Description of Artificial Sequence Synthetic polypeptide
MGFGLSWLFLVAILKGVQCQVQLVQSGAEVKKPGSSVKVSCKASGGTFSSYAISWVRQAPGQGLEWMGSIIPIFGTVNYAQKFQGRVTITADESTSTAYMELSSLRSEDTAVYYCARDPSVNLYWYFDLWGRGTLVTVSS

SEQ ID NO: 20 - protein, 127 aa, Artificial Sequence
  Description of Artificial Sequence Synthetic polypeptide
MGTPAQLLFLLLLWLPDTTGDIQMTQSPSTLSASVGDRVTITCRASQSISSWWAWYQQKPGKAPKLLIYDASSLESGVPSRFSGSGSGTEFTLTISSLQPDDFATYYCQQYHAHPTTFGGGTKVEIK

SEQ ID NO: 21 - protein, 138 aa, Artificial Sequence
  Description of Artificial Sequence Synthetic polypeptide
MGFGLSWLFLVAILKGVQCQVQLVQSGAEVKKPGSSVKVSCKASGGTFGSYAISWVRQAPGQGLEWMGSIIPIFGAANPAQKSQGRVTITADESTSTAYMELSSLRSEDTAVYYCAKMGRGKVAFDIWGQGTMVTVSS

SEQ ID NO: 22 - protein, 133 aa, Artificial Sequence
  Description of Artificial Sequence Synthetic polypeptide
MGTPAQLLFLLLLWLPDTTGDIVMTQSPDSLAVSLGERATINCKSSQSVLYSPNNKNYLAWYQQKPGQPPKLLIYWASTRESGVPDRFSGSGSGTDFTLTISSLQAEDVAVYYCQQYYGSPITFGGGTKVEIK

SEQ ID NO: 23 - protein, 142 aa, Artificial Sequence
  Description of Artificial Sequence Synthetic polypeptide
MGFGLSWLFLVAILKGVQCQVQLVQSGAEVKKPGASVKVSCKASGYAFTSYGINWVRQAPGQGLEWMGWISAYNGNTYYAQKLRGRVTMTTDTSTSTAYMELRSLRSDDTAVYYCARDLGGYGSGSVPFDPWGQGTLVTVSS

SEQ ID NO: 24 - protein, 128 aa, Artificial Sequence
  Description of Artificial Sequence Synthetic polypeptide
MGTPAQLLFLLLLWLPDTTGEIVMTQSPATLSVSPGERATLSCRASQSVSSNLAWYQQKPGQAPRLLIYGASTRATGIPARFSGSGSGTEFTLTISSLQSEDFAVYYCQDYRTWPRRVFGGGTKVEIK

SEQ ID NO: 25 - protein, 451 aa, Artificial Sequence
  Description of Artificial Sequence Synthetic polypeptide
QVQLVQSGAEVKKPGSSVKVSCKASGGTFSSYAISWVRQAPGQGLEWMGSIIPIFGTVNYAQKFQGRVTITADESTSTAYMELSSLRSEDTAVYYCARDPSVNLYWYFDLWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 26 - protein, 214 aa, Artificial Sequence
  Description of Artificial Sequence Synthetic polypeptide
DIQMTQSPSTLSASVGDRVTITCRASQSISSWWAWYQQKPGKAPKLLIYDASSLESGVPSRFSGSGSGTEFTLTISSLQPDDFATYYCQQYHAHPTTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 27 - protein, 651 aa, Artificial Sequence
  Description of Artificial Sequence Synthetic polypeptide
MRPSGTAGAALLALLAALCPASRALEEKKVCQGTSNKLTQLGTFEDHFLSLQRMFNNCEVVLGNLEITYVQRNYDLSFLKTIQEVAGYVLIALNTVERIPLENLQIIRGNMYYENSYALAVLSNYDANKTGLKELPMRNLQEILHGAVRFSNNPALCNVESIQWRDIVSSDFLSNMSMDFQNHLGSCQKCDPSCPNGSCWGAGEENCQKLTKIICAQQCSGRCRGKSPSDCCHNQCAAGCTGPRESDCLVCRKFRDEATCKDTCPPLMLYNPTTYQMDVNPEGKYSFGATCVKKCPRNYVVTDHGSCVRACGADSYEMEEDGVRKCKKCEGPCRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSHHHHHH

SEQ ID NO: 28 - DNA, 20 bp, Artificial Sequence
  Description of Artificial Sequence Synthetic primer
aagatgggggaaagaagagc

SEQ ID NO: 29 - DNA, 20 bp, Artificial Sequence
  Description of Artificial Sequence Synthetic primer
gcatgctccagactgccttg

SEQ ID NO: 30 - DNA, 20 bp, Artificial Sequence
  Description of Artificial Sequence Synthetic primer
aagatgggggaaagaagagc

SEQ ID NO: 31 - DNA, 20 bp, Artificial Sequence
  Description of Artificial Sequence Synthetic primer
gcatgctccagactgccttg

SEQ ID NO: 32 - DNA, 9549 bp, Artificial Sequence
  Description of Artificial Sequence Synthetic polynucleotide
cccggggttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactgtcgggatcaacaagtttgtacaaaaaagctgaacgagaaacgtaaaatgatataaatatcaatatattaaattagattttgcataaaaaacagactacataatactgtaaaacacaacatatccagtcactatggcggccgcattaggcaccccaggctttacactttatgcttccggctcgtataatgtgtggattttgagttaggatccgtcgagattttcaggagctaaggaagctaaaatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaattccgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtttgtgatggcttccatgtcggcagaatgcttaatgaattacaacagtactgcgatgagtggcagggcggggcgtaaagatctggatccggcttactaaaagccagataacagtatgcgtatttgcgcgctgatttttgcggtataagaatatatactgatatgtatacccgaagtatgtcaaaaagaggtatgctatgaagcagcgtattacagtgacagttgacagcgacagctatcagttgctcaaggcatatatgatgtcaatatctccggtctggtaagcacaaccatgcagaatgaagcccgtcgtctgcgtgccgaacgctggaaagcggaaaatcaggaagggatggctgaggtcgcccggtttattgaaatgaacggctcttttgctgacgagaacaggggctggtgaaatgcagtttaaggtttacacctataaaagagagagccgttatcgtctgtttgtggatgtacagagtgatattattgacacgcccgggcgacggatggtgatccccctggccagtgcacgtctgctgtcagataaagtctcccgtgaactttacccggtggtgcatatcggggatgaaagctggcgcatgatgaccaccgatatggccagtgtgccggtctccgttatcggggaagaagtggctgatctcagccaccgcgaaaatgacatcaaaaacgccattaacctgatgttctggggaatataaatgtcaggctcccttatacacagccagtctgcaggtcgaccatagtgactggatatgttgtgttttacagtattatgtagtctgttttttatgcaaaatctaatttaatatattgatatttatatcattttacgtttctcgttcagctttcttgtacaaagtggtttagtaatgagctagcgctaaccggtggcgcgttaagtcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcgtcgactttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtacgtatagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctagggacgtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctggagctgcaagcttaatgtagtcttatgcaatactcttgtagtcttgcaacatggtaacgatgagttagcaacatgccttacaaggagagaaaaagcaccgtgcatgccgattggtggaagtaaggtggtacgatcgtgccttattaggaaggcaacagacgggtctgacatggattggacgaaccactgaattgccgcattgcagagatattgtatttaagtgcctagctcgatacataaacgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccttgcgccttttccaaggcagccctgggtttgcgcagggacgcggctgctctgggcgtggttccgggaaacgcagcggcgccgaccctgggtctcgcacattcttcacgtccgttcgcagcgtcacccggatcttcgccgctacccttgtgggccccccggcgacgcttcctgctccgcccctaagtcgggaaggttccttgcggttcgcggcgtgccggacgtgacaaacggaagccgcacgtctcactagtaccctcgcagacggacagcgccagggagcaatggcagcgcgccgaccgcgatgggctgtggccaatagcggctgctcagcagggcgcgccgagagcagcggccgggaaggggcggtgcgggaggcggggtgtggggcggtagtgtgggccctgttcctgcccgcgcggtgttccgcattctgcaagcctccggagcgcacgtcggcagtcggctccctcgttgaccgaatcaccgacctctctccccagggggtaccaccggagcttaccatgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgccgtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctgagaattctagatcttgagacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccactttggcgccggctcgagggggatgaccgagtacaagccc

SEQ ID NO: 33 - DNA, 3633 bp, Artificial Sequence
  Description of Artificial Sequence Synthetic polynucleotide
atgcgaccctccgggacggccggggcagcgctcctggcgctgctggctgcgctctgcccggcgagtcgggctctggaggaaaagaaagtttgccaaggcacgagtaacaagctcacgcagttgggcacttttgaagatcattttctcagcctccagaggatgttcaataactgtgaggtggtccttgggaatttggaaattacctatgtgcagaggaattatgatctttccttcttaaagaccatccaggaggtggctggttatgtcctcattgccctcaacacagtggagcgaattcctttggaaaacctgcagatcatcagaggaaatatgtactacgaaaattcctatgccttagcagtcttatctaactatgatgcaaataaaaccggactgaaggagctgcccatgagaaatttacaggaaatcctgcatggcgccgtgcggttcagcaacaaccctgccctgtgcaacgtggagagcatccagtggcgggacatagtcagcagtgactttctcagcaacatgtcgatggacttccagaaccacctgggcagctgccaaaagtgtgatccaagctgtcccaatgggagctgctggggtgcaggagaggagaactgccagaaactgaccaaaatcatctgtgcccagcagtgctccgggcgctgccgtggcaagtcccccagtgactgctgccacaaccagtgtgctgcaggctgcacaggcccccgggagagcgactgcctggtctgccgcaaattccgagacgaagccacgtgcaaggacacctgccccccactcatgctctacaaccccaccacgtaccagatggatgtgaaccccgagggcaaatacagctttggtgccacctgcgtgaagaagtgtccccgtaattatgtggtgacagatcacggctcgtgcgtccgagcctgtggggccgacagctatgagatggaggaagacggcgtccgcaagtgtaagaagtgcgaagggccttgccgcaaagtgtgtaacggaataggtattggtgaatttaaagactcactctccataaatgctacgaatattaaacacttcaaaaactgcacctccatcagtggcgatctccacatcctgccggtggcatttaggggtgactccttcacacatactcctcctctggatccacaggaactggatattctgaaaaccgtaaaggaaatcacagggtttttgctgattcaggcttggcctgaaaacaggacggacctccatgcctttgagaacctagaaatcatacgcggcaggaccaagcaacatggtcagttttctcttgcagtcgtcagcctgaacataacatccttgggattacgctccctcaaggagataagtgatggagatgtgataatttcaggaaacaaaaatttgtgctatgcaaatacaataaactggaaaaaactgtttgggacctccggtcagaaaaccaaaattataagaaacagaggtgaaaacagctgcaaggccacaggccaggtctgccatgccttgtgctcccccgagggctgctggggcccggagcccagggactgcgtctcttgccggaatgtcagccgaggcagggaatgcgtggacaagtgcaaccttctggagggtgagccaagggagtttgtggagaactctgagtgcatacagtgccacccagagtgcctgcctcaggccatgaacatcacctgcacaggacggggaccagacaactgtatccagtgtgcccactacattgacggcccccactgcgtcaagacctgcccggcaggagtcatgggagaaaacaacaccctggtctggaagtacgcagacgccggccatgtgtgccacctgtgccatccaaactgcacctacggatgcactgggccaggtcttgaaggctgtccaacgaatgggcctaagatcccgtccatcgccactgggatggtgggggccctcctcttgctgctggtggtggccctggggatcggcctcttcatgcgaaggcgccacatcgttcggaagcgcacgctgcggaggctgctgcaggagagggagcttgtggagcctcttacacccagtggagaagctcccaaccaagctctcttgaggatcttgaaggaaactgaattcaaaaagatcaaagtgctgggctccggtgcgttcggcacggtgtataagggactctggatcccagaaggtgagaaagttaaaattcccgtcgctatcaaggaattaagagaagcaacatctccgaaagccaacaaggaaatcctcgatgaagcctacgtgatggccagcgtggacaacccccacgtgtgccgcctgctgggcatctgcctcacctccaccgtgcagctcatcacgcagctcatgcccttcggctgcctcctggactatgtccgggaacacaaagacaatattggctcccagtacctgctcaactggtgtgtgcagatcgcaaagggcatgaactacttggaggaccgtcgcttggtgcaccgcgacctggcagccaggaacgtactggtgaaaacaccgcagcatgtcaagatcacagattttgggctggccaaactgctgggtgcggaagagaaagaataccatgcagaaggaggcaaagtgcctatcaagtggatggcattggaatcaattttacacagaatctatacccaccagagtgatgtctggagctacggggtgaccgtttgggagttgatgacctttggatccaagccatatgacggaatccctgccagcgagatctcctccatcctggagaaaggagaacgcctccctcagccacccatatgtaccatcgatgtctacatgatcatggtcaagtgctggatgatagacgcagatagtcgcccaaagttccgtgagttgatcatcgaattctccaaaatggcccgcgacccccagcgctaccttgtcattcagggggatgaaagaatgcatttgccaagtcctacagactccaacttctaccgtgccctgatggatgaagaagacatggacgacgtggtggatgccgacgagtacctcatcccacagcagggcttcttcagcagcccctccacgtcaagaacgccgttattatcgagcttgagtgcaaccagcaacaattccaccgtggcttgcattgatagaaatgggctgcaaagctgcccgattaaagaggattcgttcttgcagcgatacagctcagaccccacaggcgccttgactgaggacagcatagacgacaccttcctcccagtgcctgaatacataaaccagtccgttcccaaaaggcccgctggctctgtgcagaatcctgtctatcacaatcagcctctgaaccccgcgcccagccgcgacccacactaccaggacccccacagcactgcagtgggcaaccccgagtatctcaacactgtccagcccacctgtgtcaacagcacattcgacagccctgcccactgggcccagaagggttcgcatcagatctcgttagacaaccctgactaccagcaggacttctttcccaaggaagccaagccaaatggcatctttaagggctccacagctgaaaatgcagaatacctaagggtcgcgccacaatcgtccgagttcataggtgcttga

SEQ ID NO: 34 - DNA, 115 bp, Artificial Sequence
  Description of Artificial Sequence Synthetic polynucleotide
caactccataaactaaacagaaagcggtgacttactgcagctgttttcacctctgtttcttataattttggttttctgaccggaggtcccaaacagttttttccagtttattgta

SEQ ID NO: 35 - DNA, 101 bp, Artificial Sequence
  Description of Artificial Sequence Synthetic polynucleotide
ccaaacagttttttccagtttattgtatttgcatagcacaaatttttgtttcttgaaattatcacatctccatcacttatctccttgagcgagcgtaatcc

SEQ ID NO: 36 - DNA, 22 bp, Artificial Sequence
  Description of Artificial Sequence Synthetic primer
gggttcgcatcagatctcgtta

SEQ ID NO: 37 - DNA, 22 bp, Artificial Sequence
  Description of Artificial Sequence Synthetic primer
tcaagcacctatgaactcggac

SEQ ID NO: 38 - DNA, 20 bp, Artificial Sequence
  Description of Artificial Sequence Synthetic oligonucleotide
aattttggttttctgaccgg

SEQ ID NO: 39 - DNA, 20 bp, Artificial Sequence
  Description of Artificial Sequence Synthetic oligonucleotide
acggaggctaagcgtcgcaa

SEQ ID NO: 40 - DNA, 20 bp, Artificial Sequence
  Description of Artificial Sequence Synthetic oligonucleotide
ctccatcacttatctccttg

SEQ ID NO: 41 - DNA, 20 bp, Artificial Sequence
  Description of Artificial Sequence Synthetic oligonucleotide
gctatgcaaatacaataaac

SEQ ID NO: 42 - DNA, 20 bp, Artificial Sequence
  Description of Artificial Sequence Synthetic oligonucleotide
acggaggctaagcgtcgcaa

SEQ ID NO: 43 - DNA, 20 bp, Artificial Sequence
  Description of Artificial Sequence Synthetic oligonucleotide
cgcttccgcggcccgttcaa